SEQUENCE LISTING (2 sequences)

SEQ ID NO: 1 - DNA, 20 bp, Artificial Sequence
  Oligonucleotide Primer
tccgtcatcgctcctcaggg

SEQ ID NO: 2 - DNA, 20 bp, Artificial Sequence
  Oligonucleotide Primer
atgcattctgcccccaagga

(19) United States
(12) Patent Application Publication
(10) Pub. No.: US 2004/0014957 A1
(43) Pub. Date: Jan. 22, 2004

(54) Oligonucleotides having modified nucleoside units

(76) Inventors: Anne Eldrup, Encinitas, CA (US); Phillip Dan Cook, Fallbrook, CA (US); B. Lynne Parshall, Carlsbad, CA (US)

Correspondence Address: WOODCOCK WASHBURN LLP, ONE LIBERTY PLACE - 46TH FLOOR, PHILADELPHIA, PA, 19103, US

(21) Appl. No.: 10/444,628

(22) Filed: May 23, 2003

Related U.S. Application Data
(60) Provisional application No. 60/383,438, filed on May 24, 2002.

(51) Int. Cl. C07H 21/02; C07H 19/04; C07H 19/20; C07H 19/00; C07H 19/048; C 07D 4 5/02; C07D 487/02

(52) U.S. Cl. 536/23.1; 536/26.7; 536/27.13; 536/28.4; 536/28.3; 536/28.1; 544/182; 544/262; 544/310; 544/350

(57) ABSTRACT

Disclosed are oligonucleotides and oligonucleosides that include one or more modified nucleoside units. The oligonucleotides and oligonucleosides are particularly useful as antisense agents, ribozymes, aptamer, siRNA agents, probes and primers or, when hybridized to an RNA, as a substrate for RNA cleaving enzymes including RNase H and dsRNase.

### FIELD OF INVENTION

The present invention provides oligonucleotides that have one or more modified nucleoside units. The improved oligonucleotides are useful as therapeutic or prophylactic antisense agents, as ribozymes, as aptamers or as substrates for RNA cleaving enzymes including RNase H and dsRNase including siRNA oligonucleotides. The oligonucleotides of the invention are usable as a single stranded structure or in dual stranded structures, e.g., as both an antisense strand and a sense strand. Further they can be used in diagnostics or as research reagents including uses as probes and primers. The modified oligomeric compounds of the invention exhibit improved properties including binding affinity to target RNA. U.S. application No. 60/383,438, from which priority is claimed, is incorporated herein by reference.

### BACKGROUND OF THE INVENTION

Efficacy and sequence specific behavior of antisense oligonucleotides (ONs) in biological systems depend upon their resistance to enzymatic degradation. It is therefore essential, when designing potent antisense drugs, to combine features such as high binding affinity and mismatch sensitivity with nuclease resistance. Unmodified phosphodiester antisense oligonucleotides are degraded rapidly in biological fluids containing hydrolytic enzymes (Shaw, J. P.; Kent, K.; Bird, J.; Fishback, J.; Froehler, B. Nucleic Acids Res. 1991, 19, 747-750; Woolf, T. M.; Jennings, C. G. B.; Rebagliati, M; Melton, D. A. Nucleic Acids Res. 1990, 18, 1763-1769), and the first generation of modified antisense oligonucleotide drugs, such as 2′-deoxyphosphorothioate oligonucleotides, are also subject to enzymatic degradation (Maier, M.; Bleicher, K.; Kalthoff, H.; Bayer, E. Biomed. Pept., Proteins Nucleic Acids 1995, 1, 235-241; Agrawal, S.; Temsamani, J.; Tang, J. Y. Proc. Natl. Acad. Sci. 1991, 88, 7595-7599). Extensive stability against the various nucleases present in biological systems can best be achieved by modified oligonucleotides. Since 3′exonuclease activity is predominantly responsible for enzymatic degradation in serum-containing medium and in various eukaryotic cell lines, modifications located at the 3′-terminus significantly contribute to the nuclease resistance of an oligonucleotide (Shaw, J.-P.; Kent, K.; Bird, J.; Fishback, J.; Froehler, B. Nucleic Acids Res. 1991, 19, 747-750; Maier, M.; Bleicher, K.; Kalthoff, H.; Bayer, E. Biomed Pept., Proteins Nucleic Acids 1995, 1, 235-241).

The sugar moiety of nucleosides has also been extensively studied to evaluate the effect its modification has on the properties of oligonucleotides relative to unmodified oligonucleotides. The 2′-position of a ribosyl sugar moiety is one of the most studied sites for modification. Certain 2′-substituent groups have been shown to increase the lipophilicity and enhance properties such as binding affinity to target RNA, chemical stability and nuclease resistance of oligonucleotides. Many of the modifications at the 2′-position that show enhanced binding affinity also force the sugar ring into the C₃-endo conformation.

One 2′-substituent group that has been shown to enhance the properties of oligonucleotides for antisense applications is the 2′—O—CH₂CH₂—O—CH₃ (2′-O—MOE). This modification in phosphodiester ONs offers about a 2° C. increase in tm/modification relative to 2′-deoxyphosphorothioate ONs. A phosphodiester ON modified with a 2′-O—MOE has about the same nuclease resistance as a 2′-deoxyphosphorothioate ON as shown by the half-life of the full-length oligonucleotide, t_{1/2}.

Although the 2′-position is a commonly used position for antisense applications, modifications of the 3′ and 5′terminal hydroxyls of an oligonucleotide have also been shown to be advantageous sites for modifications. Oligonucleotides bearing conjugate groups at these positions have shown improved pharmacokinetic and biodistribution properties including enhanced protein binding.

Phosphodiester ON and phosphorothioate ON each have unique organ distributions and well as serum binding properties. Substituent groups at the 2′, 3′ and 5′positions also modify the particular properties of an oligonucleotide.

Accordingly, it is the object of this invention to provide oligonucleotides having novel nucleoside units incorporated in the oligonucleotide for modulating the properties of the particular oligonucleotides.

It is also the object of this invention to provide oligonucleosides that exhibit high binding affinity to target RNA.

Additional objects, advantages and novel features of this invention will become apparent to those skilled in the art upon examination of the following descriptions and claims, which are not intended to be limiting.

### SUMMARY OF INVENTION

The present invention relates to compounds that comprise a plurality of linked nucleoside units, at least one of said nucleoside units comprising a modified nucleoside of stuctural formula I including the indicated stereochemical configuration:

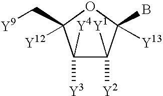

wherein B is selected from the group consisting of

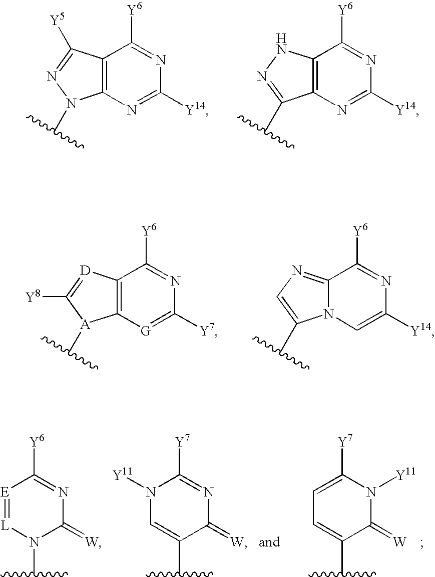

A is CH, and G is N or CH, and D is N, CH, C—CN, C—NO₂, C—C₁₋₃ alkyl, C—NHCONH₂, C—CONY¹¹Y¹¹, C—CSNY¹¹Y¹¹, C—COOY¹¹, C-hydroxy, C—C₁₋₃ alkoxy, C-amino, C—C₁₋₄ alkylamino, C-di(C₁₋₄alkyl)amino, C-halogen, C-(1,3-oxazol-2-yl), C-(1,3-thiazol-2-yl), or C-(imidazol-2-yl); wherein alkyl is unsubstituted or substituted with one to three groups independently selected from halogen, amino, hydroxy, carboxy, or C₁₋₃ alkoxy; or

A is N, and G is CH, and D is CH, C—CN, C—NO₂, C—C₁₋₃ alkyl, C—NHCONH₂, C—CONY¹¹Y¹¹, C—CSNY¹¹Y¹¹, C—COOY¹¹, C-hydroxy, C—C₁₋₃ alkoxy, C-amino, C—C1-4 alkylamino, C-di(C₁₋₄ alkyl)amino, C-halogen, C-(1,3-oxazol-2-yl), C-(1,3-thiazol-2-yl), or C-(imidazol-2-yl); wherein alkyl is unsubstituted or substituted with one to three groups independently selected from halogen, amino, hydroxy, carboxy, or C₁₋₃ alkoxy;

E is N and L is CY⁵; or E is CY⁵ and L is N;

W is O or S;

Y¹, Y², Y³ and Y⁴ each independently are a linkage to a further of said nucleoside units of said compound; hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl;

Y¹⁴ is H, CF₃, C₁₋₄ alkyl, amino, C₁₋₄ alkylamino, C₃₋₆ cycloalkylamino, or di(C₁₋₄ alkyl)amino; and

at least one of Y¹, Y², Y³, Y⁴ or Y⁹ is a linkage to a further of said nucleoside units of said compound.

Certain particularly preferred compounds of the invention, where the variables Y¹ through Y¹⁴ are as described above, include oligonucleotides and oligonucleosides wherein at least one of the nucleoside units is a nucleoside of the structure:

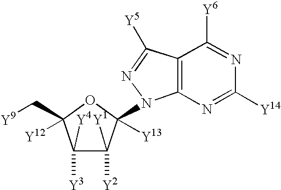

Other particularly preferred compounds of the invention, where the variables Y¹ through Y¹⁴ are as described above, include oligonucleotides and oligonucleosides wherein at least one of the nucleoside units is a nucleoside of the structure:

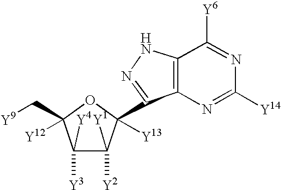

Other particularly preferred compounds of the invention, where the variables Y¹ through Y¹⁴ are as described above, include oligonucleotides and oligonucleosides wherein at least one of the nucleoside units is a nucleoside of the structure:

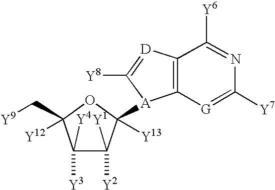

Other particularly preferred compounds of the invention, where the variables Y¹ through Y¹⁴ are as described above, include oligonucleotides and oligonucleosides wherein at least one of the nucleoside units is a nucleoside of the structure:

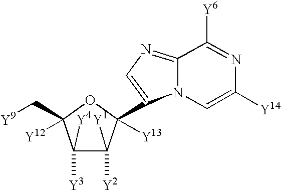

Other particularly preferred compounds of the invention, where the variables Y¹ through Y¹⁴ are described above, include oligonucleotides and oligonucleosides wherein at least one of the nucleoside units is a nucleoside of the structure:

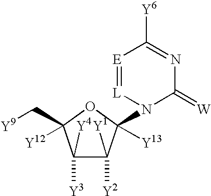

Other particularly preferred compounds of the invention, where the variables Y¹ through Y¹⁴ are described above, include oligonucleotides and oligonucleosides wherein at least one of the nucleoside units is a nucleoside of the structure:

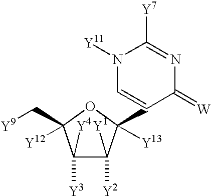

Other particularly preferred compounds of the invention, where the variables Y¹ through Y¹⁴ are described above, include oligonucleotides and oligonucleosides wherein at least one of the nucleoside units is a nucleoside of the structure:

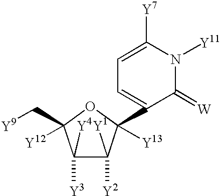

This invention further provides a compound comprising a plurality of linked nucleoside units, at least one of said nucleoside units comprising a modified nucleoside of structural formula I of the indicated stereochemical configuration:

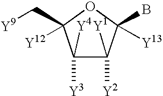

wherein B is selected from the group consisting of

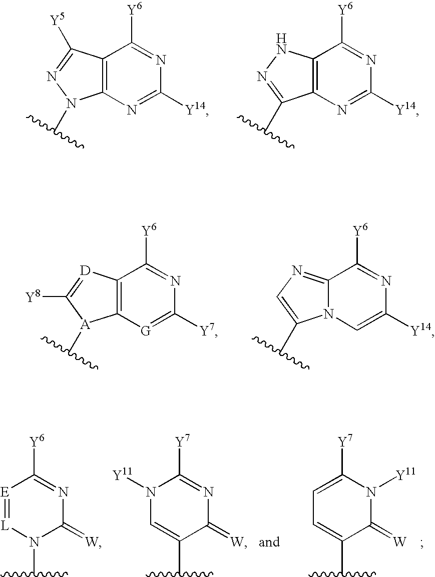

A is N or CH;

G is N or CH;

D is N;

E is N or CY⁵;

L is N or CY⁵;

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino, C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl; and

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio;; or Y¹² and Y² together with the carbon atom to which they are attached form a 3- to 6-membered saturated monocyclic ring system optionally containing a heteroatom selected from O, S, and NC0-4 alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Certain perferred compounds of the invention include compounds that comprise a plurality of linked nucleoside units, at least one of said nucleoside units comprising a modified nucleoside of the structures:

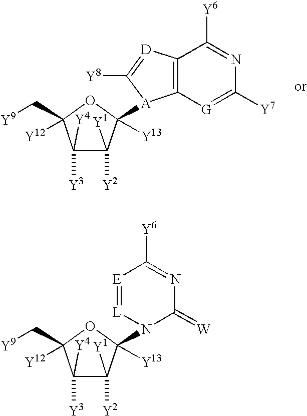

wherein

A is N or CH;

G is N or CH;

D is N;

E is N or CY⁵;

L is N or CY⁵;

W is O is S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl; and

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Further preferred compounds of the invention include compounds that comprise a plurality of linked nucleoside units, at least one of said nucleoside units comprising a modified nucleoside of the structure:

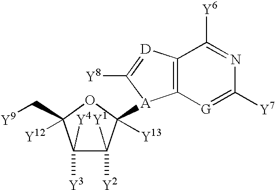

wherein

A is N or CH;

G is N or CH;

D is N;

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl; and

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Additional preferred compounds of the invention include compounds that comprise a plurality of linked nucleoside units, at least one of said nucleoside units comprising a modified nucleoside of the structure:

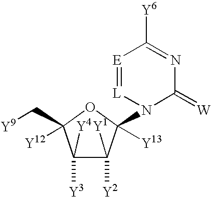

wherein

E is N or CY⁵;

L is N or CY⁵;

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y9 is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl; and

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Particularly preferred compounds are compounds where one of Y¹ and Y² is methyl and the other of Y¹ and Y² is hydroxyl or halogen.

For the sake of simplicity of the above structures, certain of the structure illustrate only one of various possible tautomeric forms of the particular structure. This invention is not meant to be limited to only the illustrated tautomeric form but should be construed to include other of the possible tautomeric forms of these structures.

In the above compounds wherein one or more of Y¹, Y², Y³, Y⁴ or Y⁹ is a linkage to a further of said nucleoside units of said compound, in one embodiment of the invention the linkages can include a phosphorous atom. Such compounds comprise an oligonucleotide. In preferred oligonucleotide compounds of the invention the plurality of nucleoside units are linked together in the oligonucleotide by phosphodiester, phosphorothioate, chiral phosphorothioate, phosphorodithioate, phosphotriester, aminoalkylphosphotriester, methyl or alkyl phosphonate, 3′-alkylene phosphonate, 5′-alkylene phosphonate, chiral phosphonate, phosphinate, 3′-amino phosphoramidate, aminoalkylphosphoramidate, thionophosphoramidate, thionoalkyl-phosphonate, thionoalkylphosphotriester, selenophosphates or boranophosphate linkages.

In the above compounds wherein one of Y¹, Y², Y³, Y⁴ or Y⁹ is a linkage to a further of said nucleoside units of said compound, in a further embodiment of the invention the linkages can include carbon, sulfur, oxygen, nitrogen or silicon atoms or combinations thereof. Such compounds comprise an oligonucleosides. In preferred oligonucleoside compounds of the invention the plurality of nucleoside units are linked together in the oligonucleoside by morpholino, siloxane, sulfide, sulfoxide, sulfone; formacetal, thioformacetal, methylene formacetal, methylene thioformacetal, riboacetal, alkene, sulfamate, methyleneimino, methylenehydrazino, sulfonate, sulfonamide or amide linkages.

In other embodiments of the invention, some of the nucleoside units can be linked by phosphorous atoms plus other heteroatoms, as for example, a morpholino linkage that includes both of these types of atoms. In even additional embodiments of the invention, some nucleoside units can be linked by phosphorous containing linkages and some by other hetero atom linkages forming a compound having both oligonucleotide and oligonucleoside parts thereof.

Further preferred compounds of the invention include one or more nucleoside linked together with inverted internucleotide linkages that are 3′ to 3′ or 5′ to 5′ linkages. Preferred of these inverted polarity linkages are single 3′ to 3′ linkage at the 3′-most internucleotide linkage of said compound.

Other preferred compounds of the invention include a plurality of linked nucleoside units linked together to form a chimeric oligonucleotide having a first region capable of serving as a substrate for an RNA cleaving enzyme and a second region containing said nucleoside of structural formula I. Preferred are compounds where the RNA cleaving enzyme is an RNase H enzyme or a dsRNase enzyme.

Additional preferred compounds of the invention include at least one nucleosides described above and at least one further 2′-deoxynucleoside or 2′-ribonucleoside, i.e., 2′-H or 2′-OH nucleosides. Other preferred compounds include at least one nucleoside described above and at least one further nucleoside that is a nucleoside having a 2′ substituent group and wherein said substituent group is C₁-C₂₀ alkyl, C₂-C₂₀ alkenyl, C₂-C₂₀ alkynyl, C₅-C₂₀ aryl, —O-alkyl, —O-alkenyl, —O-alkynyl O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—R), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—R), sulfone (—S(═O)₂—R), disulfide (—S—S—R), silyl, heterocycle, carbocycle, intercalator, reporter group, conjugate, polyamine, polyamide, polyalkylene glycol, and polyethers of the formula (—O-alkyl)ₘ, where m is 1 to about 10; wherein each R is, independently, hydrogen, a protecting group or substituted or unsubstituted alkyl, alkenyl, or alkynyl wherein said substituted alkyl, alkenyl, or alkynyl are substituted with haloalkyl, alkenyl, alkoxy, thioalkoxy, haloalkoxy, aryl groups as well as halogen, hydroxyl, amino, azido, carboxy, cyano, nitro, mercapto, sulfides, sulfones, or sulfoxides. A particularly preferred 2′ substituent group is the group —O—CH₂—CH₂—O—CH₃.

The oligonucleotide and oligonucleoside compounds of the present invention are particularly useful as antisense oligonucleotides and oligonucleosides, which are oligonucleotides and oligonucleosides targeted to a nucleic acid encoding a gene and which modulate the expression of that gene.

Pharmaceutical and other compositions comprising the compounds of the invention are also provided. Further provided are methods of modulating the expression of a gene in cells or tissues comprising contacting said cells or tissues with one or more of the oligonucleotide or oligonucleoside compounds or compositions of the invention. Further provided are methods of treating an animal, particularly a human, suspected of having or being prone to a disease or condition associated with expression of a gene by administering a therapeutically or prophylactic ally effective amount of one or more of the oligonucleotide compounds or compositions of the invention.

The oligonucleotides and oligonucleosides of the invention are also useful for use related to RNAi. For use related to RNAi preferred forms of oligomeric compound of the invention include a single-stranded antisense oligonucleotide that binds in a RISC complex, a double antisense/sense pair of oligonucleotide or a single strand oligonucleotide that includes both an antisense portion and a sense portion. Each of these compounds or compositions is used to induce potent and specific modulation of gene function. Such specific modulation of gene function has been shown in many species by the introduction of double-stranded structures, such as double-stranded RNA (dsRNA) molecules and has been shown to induce potent and specific antisense-mediated reduction of the function of a gene or its associated gene products. This phenomenon occurs in both plants and animals and is believed to have an evolutionary connection to viral defense and transposon silencing. Particularly preferred are oligonucleotides or oligonucleosides used as a siRNA molecule having first and second strands, at least one of said strands comprising one of the compounds as described above.

### DESCRIPTION OF PREFERRED EMBODIMENTS

The present invention relates to oligonucleotides and oligonucleosides that include at least one modified nucleoside unit. Oligonucleotides and oligonucleosides of the invention having modified nucleoside units are useful as antisense oligonucleotides, ribozymes, aptamers, for use as siRNAs, as diagnostic and research reagents and as probe and primers especially RT-PCR probes and primers.

Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of the oligonucleotides of the invention, the present invention comprehends other oligonucleotide compounds useful in other applications, including but not limited to oligonucleotide mimetics such as are described below. The oligonucleotides compounds in accordance with this invention preferably comprise from about 8 to about 80 nucleobases (i.e. from about 8 to about 80 linked nucleosides or nucleoside units). Particularly preferred are antisense oligonucleotides from about 12 to 50 nucleobases, even more preferably those comprising from about 15 to about 30 nucleobases. Antisense oligonucleotides include ribozymes, external guide sequence (EGS) oligonucleotides (oligozymes), and other short catalytic RNAs or catalytic oligonucleotides that hybridize to the target nucleic acid and modulate its expression including siRNAs.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage. Oligonucleotides have also been linked 240 to 5′.

Oligonucleotide and oligonucleoside compounds of the invention include at least one modified nucleoside unit of structural formula I of the indicated stereochemical configuration:

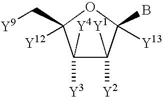

wherein B is selected from the group consisting of

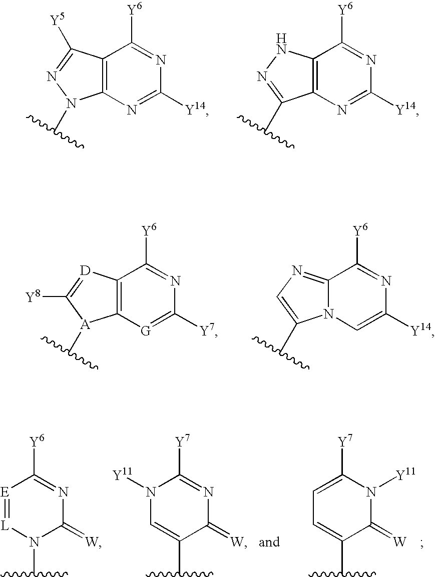

A is CH, and G is N or CH, and D is N, CH, C—CN, C—NO₂, C—C₁₋₃ alkyl, C—NHCONH₂, C—CONY¹¹Y¹¹, C—CSNY¹¹Y¹¹, C—COOY ¹¹, C-hydroxy, C—C₁₋₃ alkoxy, C-amino, C—C₁₋₄ alkylamino, C-di(C₁₋₄ alkyl)amino, C-halogen, C-(1,3-oxazol-2-yl), C-(1,3-thiazol-2-yl), or C-(imidazol-2-yl); wherein alkyl is unsubstituted or substituted with one to three groups independently selected from halogen, amino, hydroxy, carboxy, or C₁₋₃ alkoxy; or

A is N, and G is CH, and D is CH, C—CN, C—NO₂, C—C₁₋₃ alkyl, C—NHCONH₂, C—CONY¹¹Y¹¹, C—CSNY¹¹Y¹¹, C—COOY ¹¹, C-hydroxy, C—C₁₋₃ alkoxy, C-amino, C—C₁₋₄ alkylamino, C-di(C₁₋₄ alkyl)amino, C-halogen, C-(1,3-oxazol-2-yl), C-(1,3-thiazol-2-yl), or C-(imidazol-2-yl); wherein alkyl is unsubstituted or substituted with one to three groups independently selected from halogen, amino, hydroxy, carboxy, or C₁₋₃ alkoxy;

E is N and L is CY⁵; or E is CY⁵ and L is N;

W is O or S;

Y¹, Y², Y³ and Y⁴ each independently are a linkage to a further of said nucleoside units of said compound; hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or P(═W)O₃H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen, C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl;

Y¹⁴ is H, CF₃, C₁₋₄ alkyl, amino, C₁₋₄ alkylamino, C₃₋₆ cycloalkylamino, or di(C₁₋₄ alkyl)amino; and

at least one of Y¹, Y², Y³, Y⁴ or Y⁹ is a linkage to a further of said nucleoside units of said compound.

Particularly preferred compounds of the invention include oligonucleotides and oligonucleosides wherein at least one of the nucleoside units is a nucleoside of the structure:

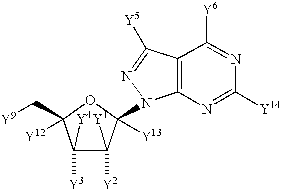

where

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C 14 alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Further particularly preferred compounds of the invention include oligonucleotides and oligonucleosides wherein at least one of the nucleoside units is a nucleoside of the structure:

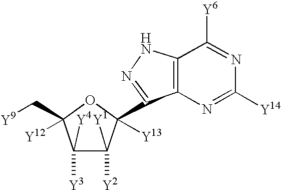

where

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Further particularly preferred compounds of the invention include oligonucleotides and oligonucleosides wherein at least one of the nucleoside units is a nucleoside of the structure:

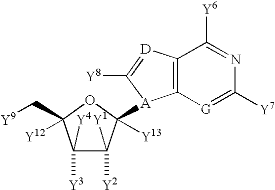

where

A is CH or N;

G is CH or N;

D is N;

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Even further particularly preferred compounds of the invention include oligonucleotides and oligonucleosides wherein at least one of the nucleoside units is a nucleoside of the structure:

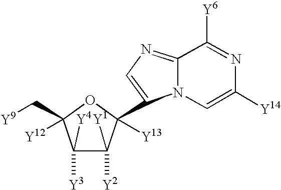

where

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C-₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Further particularly preferred compounds of the invention include oligonucleotides and oligonucleosides wherein at least one of the nucleoside units is a nucleoside of the structure:

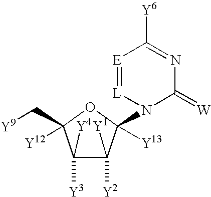

where

E is N or CY⁵;

L is N or CY⁵;

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Even further particularly preferred compounds of the invention include oligonucleotides and oligonucleosides wherein at least one of the nucleoside units is a nucleoside of the structure:

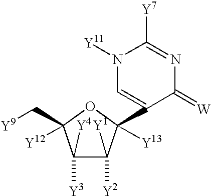

where

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

Further particularly preferred compounds of the invention include oligonucleotides and oligonucleosides wherein at least one of the nucleoside units is a nucleoside of the structure:

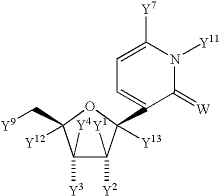

where

W is O or S;

Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C-₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;

one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;

Y⁵ is H, C₁₋₆ alkyl, C₂₋₆ alkenyl, C₂₋₆ alkynyl, C₁₋₄ alkylamino, CF₃, and halogen;

Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;

Y⁷ is hydrogen, amino, C₁₋₄ alkylamino, C₃₋₆ cycloalkylamino, or di(C₁₋₄ alkyl)amino;

Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;

Y⁹ is O—Y¹⁰, hydroxy, or O—P(═W)O₂H₂, or a linkage to a further of said nucleoside units of said compound;

Y¹⁰ is a conjugate molecule or a reporter molecule;

each Y¹¹ is independently H or C₁₋₆ alkyl;

Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and

Y¹⁴ is H, CF₃, C₁₋₄ alkyl, amino, C₁₋₄ alkylamino, C₃₋₆ cycloalkylamino, or di(C₁₋₄ alkyl)amino.

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, and the like.

The term “alkenyl” shall mean straight or branched chain alkenes of two to six total carbon atoms, or any number within this range (e.g., ethenyl, propenyl, butenyl, pentenyl, etc.).

The term “alkynyl” shall mean straight or branched chain alkynes of two to six total carbon atoms, or any number within this range (e.g., ethynyl, propynyl, butynyl, pentynyl, etc.).

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl, or cyclooctyl).

The term “cycloheteroalkyl” is intended to include non-aromatic heterocycles containing one or two heteroatoms selected from nitrogen, oxygen and sulfur. Examples of 4-6-membered cycloheteroalkyl include azetidinyl, pyrrolidinyl, piperidinyl, morpholinyl, thiamorpholinyl, imidazolidinyl, tetrahydrofuranyl, tetrahydropyranyl, tetrahydrothiophenyl, piperazinyl, and the like.

The term “alkoxy” refers to straight or branched chain alkoxides of the number of carbon atoms specified (e.g., C₁₋₄ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₄ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₄ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO2—), ethylsulfonyl, isopropylsulfonyl, etc.].

The term “alkyloxycarbonyl” refers to straight or branched chain esters of a carboxylic acid derivative of the present invention of the number of carbon atoms specified (e.g., C₁₋₄ alkyloxycarbonyl), or any number within this range [i.e., methyloxycarbonyl (MeOCO—), ethyloxycarbonyl, or butyloxycarbonyl].

The term “aryl” includes phenyl, naphthyl, and pyridyl. The aryl group is optionally substituted with one to three groups independently selected from C₁₋₄ alkyl, halogen, cyano, nitro, trifluoromethyl, C₁₋₄ alkoxy, and C₁₋₄ alkylthio.

The term “halogen” is intended to include the halogen atoms fluorine, chlorine, bromine and iodine.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally.

The term “composition”, as in “pharmaceutical composition,” is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The terms “administration of” and “administering a” compound should be understood to mean providing a compound of the invention or a prodrug of a compound of the invention to the individual in need.

The terms antisense oligonucleotides is understood to mean an oligonucleotide for use in modulating the function of a nucleic acid molecule encoding a gene. This is accomplished by providing antisense compounds, which specifically hybridize with one or more nucleic acids encoding the gene.

As used herein, the terms “target nucleic acid” and “nucleic acid encoding a gene encompass DNA encoding the gene, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered with include replication and transcription. The functions of RNA to be interfered with include all vital functions such as, for example, translocation of the RNA to the site of protein translation, translocation of the RNA to sites within the cell which are distant from the site of RNA synthesis, translation of protein from the RNA, splicing of the RNA to yield one or more mRNA species, and catalytic activity which may be engaged in or facilitated by the RNA. The overall effect of such interference with target nucleic acid function is modulation of the expression of that gene.

In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding the gene. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-LUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from the gene regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. mRNA transcripts produced via the process of splicing of two (or more) mRNAs from different gene sources are known as “fusion transcripts”. It has also been found that introns can be effective, and therefore preferred, target regions for antisense compounds targeted, for example, to DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and extronic regions.

Upon excision of one or more exon or intron regions or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases, which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable.

An antisense compound is specifically hybridizable when binding of the compound to the target DNA or RNA molecule interferes with the normal function of the target DNA or RNA to cause a loss of activity, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. It is preferred that the antisense compounds of the present invention comprise at least 80% sequence complementarity with the target nucleic acid, more that they comprise 90% sequence complementarity and even more comprise 95% sequence complementarity with the target nucleic acid sequence to which they are targeted. Percent complementarity of an antisense compound with a target nucleic acid can be determined routinely using basic local alignment search tools (BLAST programs) (Altschul et al., J. Mol. Biol., 1990, 215, 403-410; Zhang and Madden, Genome Res., 1997, 7, 649-656).

Antisense and other compounds of the invention, which hybridize to the target and inhibit expression of the target, are identified through experimentation. The sites to which these antisense compounds are specifically hybridizable are herein below referred to as “preferred target regions” and are therefore preferred sites for targeting. As used herein the term “preferred target region” is defined as at least an 8-nucleobase portion of a target region to which an active antisense compound is targeted. While not wishing to be bound by theory, it is presently believed that these target regions represent regions of the target nucleic acid, which are accessible for hybridization.

Target regions 8-80 nucleobases in length comprising a stretch of at least eight (8) consecutive nucleobases selected from within the illustrative preferred target regions are considered to be suitable preferred target regions as well.

Exemplary good preferred target regions include DNA or RNA sequences that comprise at least the 8 consecutive nucleobases from the 5′-terminus of one of the illustrative preferred target regions (the remaining nucleobases being a consecutive stretch of the same DNA or RNA beginning immediately upstream of the 5′-terminus of the target region and continuing until the DNA or RNA contains about 8 to about 80 nucleobases). Similarly good preferred target regions are represented by DNA or RNA sequences that comprise at least the 8 consecutive nucleobases from the 3′-terminus of one of the illustrative preferred target regions (the remaining nucleobases being a consecutive stretch of the same DNA or RNA beginning immediately downstream of the 3′-terminus of the target region and continuing until the DNA or RNA contains about 8 to about 80 nucleobases). One having skill in the art, once armed with the empirically-derived preferred target regions illustrated herein will be able, without undue experimentation, to identify further preferred target regions. In addition, one having ordinary skill in the art will also be able to identify additional compounds, including oligonucleotide probes and primers that specifically hybridize to these preferred target regions using techniques available to the ordinary practitioner in the art.

The oligonucleotides of invention therefore will be of a size of 8 to 80 nucleotides long. A further preferred range of oligonucleotide size is from 12 to 50 nucleotides long. An additional preferred range of oligonucleotide size is from 15 to 30 nucleotides in length.

Oligonucleotides are commonly used as research reagents and diagnostics. For example antisense oligonucleotides, which are able to inhibit gene expression with exquisite specificity, are often used by those of ordinary skill to elucidate the function of particular genes. Oligonucleotides are also used, for example, to distinguish between functions of various members of a biological pathway. Antisense modulation has, therefore, been harnessed for research use.

For use in kits and diagnostics, the oligonucleotide compounds of the present invention, either alone or in combination with other compounds or therapeutics, can be used as tools in differential and/or combinatorial analyses to elucidate expression patterns of a portion or the entire complement of genes expressed within cells and tissues.

Expression patterns within cells or tissues treated with one or more oligonucleotide compounds are compared to control cells or tissues not treated with oligonucleotide compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U. S. A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in To, Comb. Chem. High Throughput Screen, 2000, 3, 235-41).

The specificity and sensitivity of oligonucleotides is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of oligonucleotide compounds, the present invention comprehends other oligomeric antisense compounds, including but not limited to oligonucleotide mimetics such as are described below. The antisense compounds in accordance with this invention preferably comprise from about 8 to about 80 nucleobases (i.e. from about 8 to about 80 linked nucleosides). Particularly preferred antisense compounds are antisense oligonucleotides from about 12 to about 50 nucleobases, even more preferably those comprising from about 15 to about 30 nucleobases. Antisense compounds include ribozymes, external guide sequence (EGS) oligonucleotides (oligozymes), and other short catalytic RNAs or catalytic oligonucleotides, which hybridize to the target nucleic acid and modulate its expression.

Antisense compounds 8-80 nucleobases in length comprising a stretch of at least eight (8) consecutive nucleobases selected from within the illustrative antisense compounds are considered to be suitable antisense compounds as well.

Exemplary preferred antisense compounds include DNA or RNA sequences that comprise at least the 8 consecutive nucleobases from the 5′-terminus of one of the illustrative preferred antisense compounds (the remaining nucleobases being a consecutive stretch of the same DNA or RNA beginning immediately upstream of the 5′-terminus of the antisense compound which is specifically hybridizable to the target nucleic acid and continuing until the DNA or RNA contains about 8 to about 80 nucleobases). Similarly preferred antisense compounds are represented by DNA or RNA sequences that comprise at least the 8 consecutive nucleobases from the 3′-terminus of one of the illustrative preferred antisense compounds (the remaining nucleobases being a consecutive stretch of the same DNA or RNA beginning immediately downstream of the 3′-terminus of the antisense compound which is specifically hybridizable to the target nucleic acid and continuing until the DNA or RNA contains about 8 to about 80 nucleobases). Antisense and other compounds of the invention, which hybridize to the target and inhibit expression of the target, are identified through experimentation. One having skill in the art, once armed with the this disclosure will be able, without undue experimentation, to identify preferred antisense compounds.

In many species, introduction of double-stranded RNA (dsRNA) induces potent and specific gene silencing. This phenomenon occurs in both plants and animals and has roles in viral defense and transposon silencing mechanisms. This phenomenon was originally described more than a decade ago by researchers working with the petunia flower. While trying to deepen the purple color of these flowers, Jorgensen et al. introduced a pigment-producing gene under the control of a powerful promoter. Instead of the expected deep purple color, many of the flowers appeared variegated or even white. Jorgensen named the observed phenomenon “cosuppression”, since the expression of both the introduced gene and the homologous endogenous gene was suppressed (Napoli et al., Plant Cell, 1990, 2, 279-289; Jorgensen et al., Plant Mol. Biol., 1996, 31, 957-973).

Cosuppression has since been found to occur in many species of plants, fungi, and has been particularly well characterized in Neurospora crassa, where it is known as “quelling” (Cogoni and Macino, Genes Dev. 2000, 10, 638-643; Guru, Nature, 2000,404, 804-808).

The first evidence that dsRNA could lead to gene silencing in animals came from work in the nematode, Caenorhabditis elegans. In 1995, researchers Guo and Kemphues were attempting to use antisense RNA to shut down expression of the par-1 gene in order to assess its function. As expected, injection of the antisense RNA disrupted expression of par-1, but quizzically, injection of the sense-strand control also disrupted expression (Guo and Kempheus, Cell, 1995, 81, 611-620). This result was a puzzle until Fire et al. injected dsRNA (a mixture of both sense and antisense strands) into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in first generation offspring (Fire et al., Nature, 1998, 391, 806-811).

The potency of this phenomenon led Timmons and Fire to explore the limits of the dsRNA effects by feeding nematodes bacteria that had been engineered to express dsRNA homologous to the C. elegans unc-22 gene. Surprisingly, these worms developed an unc-22 null-like phenotype (Timmons and Fire, Nature 1998, 395, 854; Timmons et al., Gene, 2001, 263, 103-112). Further work showed that soaking worms in dsRNA was also able to induce silencing (Tabara et al., Science, 1998, 282, 430-431). PCT publication WO01/48183 discloses methods of inhibiting expression of a target gene in a nematode worm involving feeding to the worm a food organism which is capable of producing a double-stranded RNA structure having a nucleotide sequence substantially identical to a portion of the target gene following ingestion of the food organism by the nematode, or by introducing a DNA capable of producing the double-stranded RNA structure (Bogaert et al., 2001)

The posttranscriptional gene silencing defined in Caenorhabditis elegans resulting from exposure to double-stranded RNA (dsRNA) has since been designated as RNA interference (RNAi). This term has come to generalize all forms of gene silencing involving dsRNA leading to the sequence-specific reduction of endogenous targeted mRNA levels; unlike co-suppression, in which transgenic DNA leads to silencing of both the transgene and the endogenous gene.

Introduction of exogenous double-stranded RNA (dsRNA) into Caenorhabditis elegans has been shown to specifically and potently disrupt the activity of genes containing homologous sequences. Montgomery et al. suggests that the primary interference effects of dsRNA are post-transcriptional; this conclusion being derived from examination of the primary DNA sequence after dsRNA-mediated interference a finding of no evidence of alterations followed by studies involving alteration of an upstream operon having no effect on the activity of its downstream gene. These results argue against an effect on initiation or elongation of transcription. Finally they observed by in situ hybridization, that dsRNA-mediated interference produced a substantial, although not complete, reduction in accumulation of nascent transcripts in the nucleus, while cytoplasmic accumulation of transcripts was virtually eliminated. These results indicate that the endogenous mRNA is the primary target for interference and suggest a mechanism that degrades the targeted mRNA before translation can occur. It was also found that this mechanism is not dependent on the SMG system, an mRNA surveillance system in C. elegans responsible for targeting and destroying aberrant messages. The authors further suggest a model of how dsRNA might function as a catalytic mechanism to target homologous mRNAs for degradation. (Montgomery et al., Proc. Natl. Acad. Sci. U S A, 1998, 95, 15502-15507).

Recently, the development of a cell-free system from syncytial blastoderm Drosophila embryos that recapitulates many of the features of RNAi has been reported. The interference observed in this reaction is sequence specific, is promoted by dsRNA but not single-stranded RNA, functions by specific mRNA degradation, and requires a minimum length of dsRNA. Furthermore, preincubation of dsRNA potentiates its activity demonstrating that RNAi can be mediated by sequence-specific processes in soluble reactions (Tuschl et al., Genes Dev., 1999, 13, 3191-3197).

In subsequent experiments, Tuschl et al, using the Drosophila in vitro system, demonstrated that 21- and 22-nt RNA fragments are the sequence-specific mediators of RNAi. These fragments, which they termed short interfering RNAs (siRNAs), were shown to be generated by an RNase III-like processing reaction from long dsRNA. They also showed that chemically synthesized siRNA duplexes with overhanging 3′ ends mediate efficient target RNA cleavage in the Drosophila lysate, and that the cleavage site is located near the center of the region spanned by the guiding siRNA. In addition, they suggest that the direction of dsRNA processing determines whether sense or antisense target RNA can be cleaved by the siRNA-protein complex (Elbashir et al., Genes Dev., 2001, 15, 188-200). Further characterization of the suppression of expression of endogenous and heterologous genes caused by the 21-23 nucleotide siRNAs have been investigated in several mammalian cell lines, including human embryonic kidney (293) and HeLa cells (Elbashir et al., Nature, 2001, 411, 494-498).

The Drosophila embryo extract system has been exploited, using green fluorescent protein and luciferase tagged siRNAs, to demonstrate that siRNAs can serve as primers to transform the target mRNA into dsRNA. The nascent dsRNA is degraded to eliminate the incorporated target mRNA while generating new siRNAs in a cycle of dsRNA synthesis and degradation. Evidence is also presented that mRNA-dependent siRNA incorporation to form dsRNA is carried out by an RNA-dependent RNA polymerase activity (RdRP) (Lipardi et al., Cell, 2001, 107, 297-307).

The involvement of an RNA-directed RNA polymerase and siRNA primers as reported by Lipardi et al. (Lipardi et al., Cell, 2001, 107, 297-307) is one of the many intriguing features of gene silencing by RNA interference; suggesting an apparent catalytic nature to the phenomenon. New biochemical and genetic evidence reported by Nishikura et al. also shows that an RNA-directed RNA polymerase chain reaction, primed by siRNA, amplifies the interference caused by a small amount of “trigger” dsRNA (Nishikura, Cell, 2001, 107,415-418).

Investigating the role of “trigger” RNA amplification during RNA interference (RNAi) in Caenorhabditis elegans, Sijen et al revealed a substantial fraction of siRNAs that cannot derive directly from input dsRNA. Instead, a population of siRNAs (termed secondary siRNAs) appeared to derive from the action of the previously reported cellular RNA-directed RNA polymerase (RdRP) on mRNAs that are being targeted by the RNAi mechanism. The distribution of secondary siRNAs exhibited a distinct polarity (5′-3′; on the antisense strand), suggesting a cyclic amplification process in which RdRP is primed by existing siRNAs. This amplification mechanism substantially augmented the potency of RNAi-based surveillance, while ensuring that the RNAi machinery will focus on expressed mRNAs (Sijen et al., Cell, 2001, 107, 465476).

Most recently, Tijsterman et al. have shown that, in fact, single-stranded RNA oligomers of antisense polarity can be potent inducers of gene silencing. As is the case for co-suppression, they showed that antisense RNAs act independently of the RNAi genes rde-1 and rde-4 but require the mutator/RNAi gene mut-7 and a putative DEAD box RNA helicase, mut-14. According to the authors, their data favor the hypothesis that gene silencing is accomplished by RNA primer extension using the mRNA as template, leading to dsRNA that is subsequently degraded suggesting that single-stranded RNA oligomers are ultimately responsible for the RNAi phenomenon (Tijsterman et al., Science, 2002, 295, 694-697).

A number of PCT applications have recently published that related to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

U.S. Pat Nos. 5,898,031 and 6,107,094, each of which is commonly owned with this application and each of which is herein incorporated by reference, describe certain oligonucleotide having RNA like properties. When hybridized with RNA, these olibonucleotides serve as substrates for a dsRNase enzyme with resultant cleavage of the RNA by the enzyme.

Antisense technology is an effective means for modulating the levels of specific gene products and may therefore prove to be uniquely useful in a number of therapeutic, diagnostic, and research applications involving gene silencing. The present invention therefore further provides oligonucleotides useful for modulating gene silencing pathways, including those involving antisense, RNA interference, dsRNA enzymes and non-antisense mechanisms. One having skill in the art, once armed with this disclosure will be able, without undue experimentation, to identify preferred oligonucleotide compounds for these uses.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure, however, open linear structures are generally preferred. In addition, linear structures may also have internal nucleobase complementarity and may therefore fold in a manner as to produce a double stranded structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Many of the modified nucleosides of the invention, by virtue of the substituent groups present on their 3′ and 5′ positions, e.g., 3′ and 5′ OH groups, will be incorporate into oligonucleotide or oligonucleoside via 3′ to 5′ linkage. Other of the modified nucleoside of the invention, by virtue of the substituent groups present on their 2′ and 5′ positions, e.g., 2′ and 5′ OH groups, will be incorporated in an oligonucleotide or oligonucleoside via a 2′ to 5′ linkage.

Specific examples of preferred antisense oligonucleotides useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. No.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetal and thioformacetal backbones; methylene formacetal and methylene thioformacetal backbones; riboacetal backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Most preferred embodiments of the invention are oligonucleotides with phosphorothioate backbones and oligonucleosides with heteroatom backbones, and in particular -CH₂—NH—O—CH₂—, —CH₂—N(CH₃)—O—CH₂- [known as a methylene (methylimino) or MMI backbone], —CH₂—O—N(CH₃)—CH₂—, —CH₂—N(CH₃)—N(CH₃)—CH₂— and -O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. 5,034,506.

In addition to the modified nucleoside units described above, other modified nucleoside units can also be incorporated in to the oligonucleotides of the invention. Such other modified nucleoside units include nucleosides having sugar substituent groups including OH; F; O—, S—, or N-alkyl; O—, S—, or N-alkenyl; O—, S—or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙOₘ[(CH₂)ₙCH₃]₂, where n and m are from 1 to about 10. Other preferred oligonucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other preferred sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-Sugar substituent groups may be in the arabino (up) position or ribo (down) position. A preferred 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula Iₐ or IIₐ:

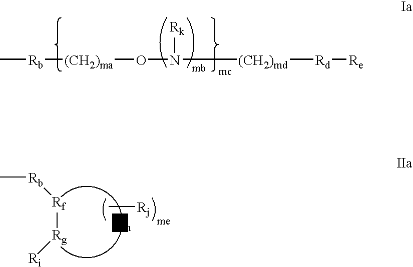

wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ;

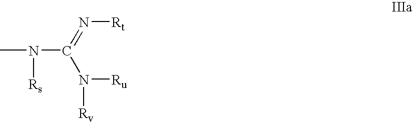

Rₚ and R_{q} are each independently hydrogen or C₁-C₁₀ alkyl;

Rᵣ is -Rₓ-R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or -Rₓ-R_{y};

Rₚ is hydrogen, a nitrogen protecting group or -Rₓ-R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z}, is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;

mₐ is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Particularly preferred sugar substituent groups include O[(CH₂)ₙO]ₘCH₃, O(CH₂)ₙOCH₃, O(CH₂)ₙNH₂, O(CH₂)ₙCH₃ O(CH₂)ₙONH₂ and O(CH₂)ₙON[(CH₂)ₙCH₃)]₂, where n and m are from 1 to about 10.

A further preferred modification of the sugar moiety is a locked nucleic acid structure (LNA) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Oligonucleotides may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1 ,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. A preferred group of conjugates are reporter molecules. Such preferred reported molecules have a physical or chemical property for identification in gels, fluids, whole cellular systems or broken cellular systems. They are capable of being identified via spectroscopy, radioactivity, colorimetric assays, fluorescence or specific binding. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application 09/334,130 (filed Jun. 15, 1999), which is incorporated herein by reference in its entirety.

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

In accordance with a further aspect of this invention, the oligonucleotides of the invention can be used in nucleic acid duplexes comprising the antisense strand oligonucleotide and its complement sense strand oligonucleotide. Either of these can be of a sequence designed to hybridize to a specific target or targets, however, normally the antisense oligonucleotide with be designed to bind to the target. The ends of the strands may be modified by the addition of one or more natural or modified nucleobases to form an overhang. The sense strand of the duplex is designed and synthesized as the complement of the antisense strand and may also contain modifications or additions to either terminus. For example, in one embodiment, both strands of the duplex would be complementary over the central nucleobases, each having overhangs at one or both termini.

For the purposes of describing an embodiment of this invention, the combination of an antisense strand and a sense strand, each of can be of a specified length, for example from 12 to 30 nucleotides long, is identified as a complementary pair of siRNA oligonucleotides. These complementary pair of siRNA oligonucleotides can include additional nucleotides on either of their 5′ or 3′ ends. Further they can include other molecules or molecular structures on their 3′ or 5′ ends such as a phosphate group on the 5′ end. A preferred group of compounds of the invention include a phosphate group on the 5′ end of the antisense strand compound. Other preferred compounds also include a phosphate group on the 5′ end of the sense strand compound. An even further preferred compounds would include additional nucleotides such as a two base overhang on the 3′ end.

For example, a preferred siRNA complementary pair of oligonucleotides comprise an antisense strand oligomeric compound having the sequence CGAGAGGCGGACGGGACCG and having a two-nucleobase overhang of deoxythymidine(dT) and its complement sense strand. These oligonucleotides would have the following structure:

| | |
|---|---|
| 5′ c g a g a g g c g g a c g g g a c c g T T 3′ | Antisense Strand |
| \| \| \| \| \| \| \| \| \| \| \| \| \| \| \| \| \| \| \| | |
| 3′ T T g c t c t c c g c c t g c c c t g g c 5′ | Complement Strand |

In an additional embodiment of the invention, a single oligonucleotide having both the antisense portion as a first region in the oligonucleotide and the sense portion as a second region in the oligonucleotide is selected. The first and second regions are linked together by either a nucleotide linker (a string of one or more nucleotides that are linked together in a sequence) or by a non-nucleotide linker region or by a combination of both a nucleotide and non-nucleotide structure. In each of these structures, the oligonucleotide, when folded back on itself, would be complementary at least between the first region, the antisense portion, and the second region, the sense portion. Thus the oligonucleotide would have a palindrome within it structure wherein the first region, the antisense portion in the 5′ to 3′ direction, is complementary to the second region, the sense portion in the 3′ to 5′ direction.

In a further embodiment, the invention includes an oligonucleotide/protein composition. This composition has both an oligonucleotide component and a protein component. The oligonucleotide component comprises at least one oligonucleotide, either the antisense or the sense oligonucleotide but preferable the antisense oligonucleotide (the oligonucleotide that is antisense to the target nucleic acid). The oligonucleotide component can also comprise both the antisense and the sense strand oligonucleotides. The protein component of the composition comprises at least one protein that forms a portion of the RNA-induced silencing complex, i.e., the RISC complex.

RISC is a ribonucleoprotein complex that contains an oligonucleotide component and proteins of the Argonaute family of proteins, among others. While we do not wish to be bound by theory, the Argonaute proteins make up a highly conserved family whose members have been implicated in RNA interference and the regulation of related phenomena. Members of this family have been shown to possess the canonical PAZ and Piwi domains, thought to be a region of protein-protein interaction. Other proteins containing these domains have been shown to effect target cleavage, including the RNAse, Dicer. The Argonaute family of proteins includes, but depending on species, are not necessary limited to, elF2C1 and elF2C2. elF2C2 is also known as human GERp95. While we do not wish to be bound by theory, at least the antisense oligonucleotide strand is bound to the protein component of the RISC complex. Additional, the complex might also include the sense strand oligonucleotide (see Carmell et al, Genes and Development 2002, 16, 2733-2742).

Also while we do not wish to be bound by theory, it is further believe that the RISC complex may interact with one or more of the translation machinery components. Translation machinery components include but are not limited to proteins that effect or aid in the translation of an RNA into protein including the ribosomes or polyribosome complex. Therefore, in a further embodiment of the invention, the oligonucleotide component of the invention is associated with a RISC protein component and further associates with the translation machinery of a cell. Such interaction with the translation machinery of the cell would include interaction with structural and enzymatic proteins of the translation machinery including but not limited to the polyribosome and ribosomal subunits.

In a further embodiment of the invention, the oligonucleotide of the invention is associated with cellular factors such as transporters or chaperones. These cellular factors can be protein, lipid or carbohydrate based and can have structural or enzymatic functions that may or may not require the complexation of one or more metal ions.

Furthermore, the oligonucleotide of the invention itself may have one or more. moieties that are bound to the oligonucleotide which facilitate the active or passive transport, localization or compartmentalization of the oligonucleotide. Cellular localization includes, but is not limited to, localization to within the nucleus, the nucleolus or the cytoplasm. Compartmentalization includes, but is not limited to, any directed movement of the oligonucleotides of the invention to a cellular compartment including the nucleus, nucleolus, mitochondrion, or imbedding into a cellular membrane surrounding a compartment or the cell itself.

In a further embodiment of the invention, the oligonucleotide of the invention is associated with cellular factors that affect gene expression, more specifically those involved in RNA modifications. These modifications include, but are not limited to posttrascriptional modifications such as methylation. Furthermore, the oligonucleotide of the invention itself may have one or more moieties that are bound to the oligonucleotide which facilitate the posttranscriptional modification.

The oligomeric compounds of the invention may be used in the form of single-stranded, double-stranded, circular or hairpin oligomeric compounds and may contain structural elements such as internal or terminal bulges or loops. Once introduced to a system, the oligomeric compounds of the invention may interact with or elicit the action of one or more enzymes or may interact with one or more structural proteins to effect modification of the target nucleic acid.

One non-limiting example of such an interaction is the RISC complex. Use of the RISC complex to effect cleavage of RNA targets thereby mediated inhibition of gene expression. Similar roles have been postulated for other ribonucleases such as those in the RNase III and ribonuclease L family of enzymes and might greatly enhances the efficiency of the oligonucleotide.

Preferred forms of oligomeric compound of the invention thus include a single-stranded antisense oligonucleotide having a mode of action via the various classical antisense mechanisms of action including but not limited to antisense oligonucleotides, ribozymes, aptamers, and also a single-stranded antisense oligonucleotide that binds in a RISC complex, a double stranded antisense/sense pair of oligonucleotide or a single strand oligonucleotide that includes both an antisense portion and a sense portion. Each of these compounds or compositions is used to induce potent and specific modulation of gene function. Such specific modulation of gene function has been shown in many species by the introduction of double-stranded structures, such as double-stranded RNA (dsRNA) molecules and has been shown to induce potent and specific antisense-mediated reduction of the function of a gene or its associated gene products. This phenomenon occurs in both plants and animals and is believed to have an evolutionary connection to viral defense and transposon silencing.

The compounds and compositions of the invention are used to modulate the expression of a target nucleic acid. “Modulators” are those oligomeric compounds that decrease or increase the expression of a nucleic acid molecule encoding a target and which comprise at least an 8-nucleobase portion that is complementary to a preferred target segment. The screening method comprises the steps of contacting a preferred target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the present invention

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder that can be treated by modulating the expression of a gene, is treated by administering antisense compounds targeted to the gene in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding a gene, enabling sandwich and other assays to easily be constructed to exploit this fact. Hybridization of the antisense oligonucleotides of the invention with a nucleic acid encoding the gene can be detected by means known in the art. Such means may include conjugation of an enzyme to the oligonucleotide, radiolabelling of the oligonucleotide or any other suitable detection means. Kits using such detection means for detecting the level of the gene in a sample may also be prepared.

The present invention also includes pharmaceutical compositions and formulations that include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Prefered bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate,. Prefered fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also prefered are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include polyamino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyomithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. applications Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999) each of which is incorporated herein by reference in their entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in- water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/olw emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered. orally as o/w emulsions.

In one embodiment of the present invention, the compositions of oligonucleotides and nucleic acids are formulated as microemulsions. A microemulsion may be defined as a system of water, oil and amphiphile that is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C 10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome that is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes that interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes that are pH-sensitive or negatively-charged entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/ cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6,466).

Liposomes also include “sterically stabilized” liposomes, a term, which as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, which contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets that are so highly deformable that they are easily able to penetrate through pores that are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, NY, 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, NY, 1988, p. 285).

In one embodiment, the present invention employs various penetration enhancers to affect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, N.Y., 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration that do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as. alcohols, or solutions of the nucleic acids in liquid or solid oil bases. The solutions may also contain buffers, diluents and other suitable additives. Pharmaceutically acceptable organic or inorganic excipients suitable for non-parenteral administration that do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

While the present invention has been described with specificity in accordance with certain of its preferred embodiments, the following examples serve only to illustrate the invention and are not intended to limit the same.

As will be recognized, the steps of certain processes of the present invention need not be performed any particular number of times or in any particular sequence. Additional objects, advantages, and novel features of this invention will become apparent to those skilled in the art upon examination of the following synthetic teachings and working examples which are intended to be illustrative of the present invention, and not limiting thereof.

### EXAMPLES

Representative Modified Nucleoside Preparation

Modified nucleoside units for incorporation in to oligonucleotides of the present invention can be prepared following synthetic methodologies well-established in the practice of nucleoside and nucleotide chemistry. Reference is made to the following text for a description of synthetic methods in nucleoside and nucleotide chemistry, which is incorporated by reference herein in its entirety: “Chemistry of Nucleosides and Nucleotides,” L. B. Townsend, ed., Vols. 1-3, Plenum Press, 1988.

A representative general method for the preparation of modified nucleosides units of use in oligonucleotides of the present invention is outlined in Scheme 1 below. This scheme illustrates the synthesis of nucleosides of structural formula 1-7 wherein the furanose ring has the β-D-ribo configuration. The starting material is a 3,5-bis-O-protected alkyl furanoside, such as methyl furanoside, of structural formula 1-1. The C-2 hydroxyl group is then oxidized with a suitable oxidizing agent, such as a chromium trioxide or chromate reagent or Dess-Martin periodinane, to afford a C-2 ketone of structural formula 1-2. Addition of a Grignard reagent, such as an alkyl, alkenyl, or alkynyl magnesium halide (for example, MeMgBr, EtMgBr, vinylMgBr, allylMgBr, and ethynylMgBr) across the carbonyl double bond of 1-2 in a suitable organic solvent, such as tetrahydrofuran, diethyl ether, and the like, affords the C-2 tertiary alcohol of structural formula 1-3. A good leaving group (such as Cl, Br, and I) is next introduced at the C-1 (anomeric) position of the furanoid sugar derivative by treatment of the furanoside of formula 1-3 with a hydrogen halide in a suitable organic solvent, such as hydrogen bromide in acetic acid, to afford the intermediate furanosyl halide 1-4. A C-1 sulfonate, such methanesulfonate (MeSO₂O—), trifluoromethanesulfonate (CF₃SO₂O—), or p-toluenesulfonate (—OTs), may also serve as a useful leaving group in the subsequent reaction to generate the glycosidic (nucleosidic) linkage. The nucleosidic linkage is constructed by treatment of the intermediate of structural formula 1-4 with the metal salt (such as lithium, sodium, or potassium) of an appropriately substituted 1H-pyrazolo[4,5-d]pyrimidine 1-5, such as an appropriately substituted 4-halo-1H-pyrazolo[4,5-d]pyrimidine, which can be generated in situ by treatment with an alkali hydride (such as sodium hydride), an alkali hydroxide (such as potassium hydroxide), an alkali carbonate (such as potassium carbonate), or an alkali hexamethyldisilazide (such as NaHMDS) in a suitable anhydrous organic solvent, such as acetonitrile, tetrahydrofuran, diethyl ether, or N,N-dimethylformamide (DMF). The displacement reaction can be catalyzed by using a phase-transfer catalyst, such as TDA-1 or triethylbenzylammonium chloride, in a two-phase system (solid-liquid or liquid-liquid). The optional protecting groups in the protected nucleoside of structural formula 1-6 are then cleaved following established deprotection methodologies, such as those described in T. W. Greene and P. G. M. Wuts, “Protective Groups in Organic Synthesis,” 3rd ed., John Wiley & Sons, 1999. Optional introduction of an amino group at the 4-position of the pyrazolo[4,5-d]pyrimidine nucleus is effected by treatment of the 4-halo intermediate 1-6 with the appropriate amine, such as alcoholic ammonia or liquid ammonia, to generate a primary amine at the C4 position (—NH₂), an alkylamine to generate a secondary amine (—NHR), or a dialkylamine to generate a tertiary amine (—NRR′). A 7H-pyrazolo[4,5-d]pyrimidin-4(3H)one compound may be derived by hydrolysis of 1-6 with aqueous base, such as aqueous sodium hydroxide. Alcoholysis (such as methanolysis) of 1-6 affords a C-4 alkoxide (—OR), whereas treatment with an alkyl mercaptide affords a C-4 alkylthio (—SR) derivative. Subsequent chemical manipulations well-known to practitioners of ordinary skill in the art of organic/medicinal chemistry may be required to attain the desired compounds of the present invention.

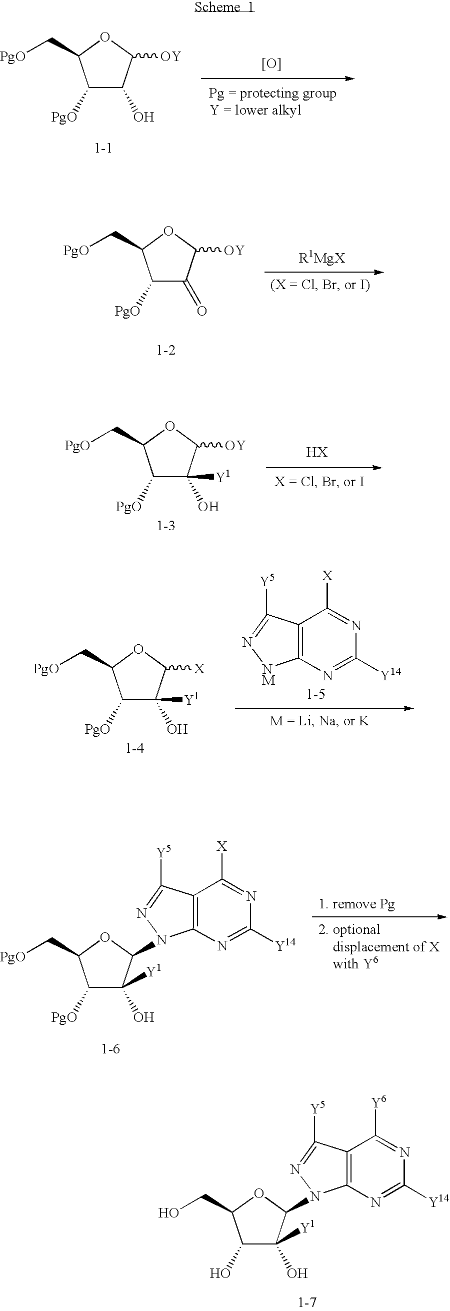

In the examples below all temperatures are degrees Celsius unless otherwise noted.

### Example 1

3′-Deoxyguanosine

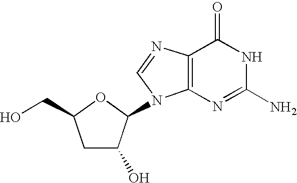

This compound was prepared following the procedures described in Nucleosides Nucleotides, 13: 1049 (1994).

### Example 2

3′-Deoxy-3′-fluoroguanosine

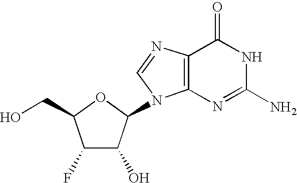

This compound was prepared following the procedures described in J. Med. Chem. 34: 2195 (1991).

### Example 3

8-Azidoguanosine

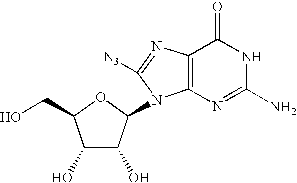

This compound was prepared following the procedures described in Chem. Pharm. Bull.16: 1616 (1968).

### Example 4

8-Bromoguanosine

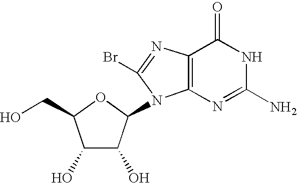

This compound was obtained from commercial sources.

### Example 5

2′-O-Methylguanosine

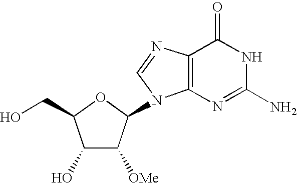

This compound was obtained from commercial sources.

### Example 6

3′-Deoxy-3′-(fluoromethyl)guanosine

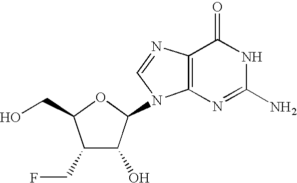

To a solution of 1,2-O-diacetyl-5-O-(p-toluoyl)-3-deoxy-3-(fluoromethyl)-D-ribofuranose (257 mg, 0.7 mmol) [prepared by a similar method as that described for the corresponding 5-O-benzyl derivative in J Med. Chem. 36: 353 (1993)] and N²-acetyl-O⁶-(diphenylcarbamoyl)guanine (554 mg, 1.43 mmol) in anhydrous acetonitrile (6.3 mL) was added bis(trimethylsilyl)acetamide (BSA) (1.03 g, 5 mmol). The reaction mixture was stirred at reflux for 30 minutes, and the bath was removed. The reaction mixture was cooled in an ice bath and TMS-triflate (288 mg, 1.3 mmol) was added with stirring. After addition was complete, the reaction was heated at reflux for 2 hr., the reaction mixture was poured onto ice and extracted with chloroform (5×10 mL). The combined organic layers were washed with aqueous saturated sodium bicarbonate, brine and dried over anhydrous Na₂SO₄. The solvent was removed under reduced pressure and the residue chromatographed over silica gel using 5% acetone/CH₂Cl₂ as the eluant to furnish the fully protected corresponding nucleoside derivative. This was dissolved in 1,4-dioxane (1.5 mL) to which was added 40% MeNH₂/H₂O (1.3 g, 17 mmol). The reaction mixture was stirred for 1 day, evaporated and the residue crystallized with ether/MeOH to provide the title compound (58 mg). ¹H NMR (DMSO-d₆): □2.76-2.67 (m, 1H); 3.55-3.50 (m, 1H), 2.76-2.67 (m, 1H); 3.71-3.66 (m, 1H), 4.08-4.04 (m, 1H), 4.77-4.50 (m, 3H), 5.06 (t, 1H, J=5.3 Hz), 5.69 (d, 1H, J=3.4 Hz), 5.86 (d, 1H, J=5.1 Hz), 6.45 (bs, 2H), 7.97 (s, 1H), 10.59 (s, 1H). ¹⁹F NMR (DMSO-d₆): □-221.46 (m, F).

### Example 7

2-Amino-3,4-dihydro4-oxo-7-(□-D-ribofuranosyl)-7H-pyrrolo[2,3-d]pyrimidin-5-carboxamide

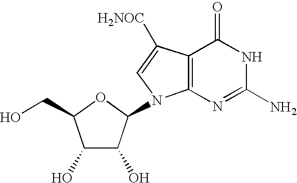

This compound was prepared following the procedures described in Tetrahedron. Lett. 25: 4793 (1983).

### Example 8

2-Amino-3,4-dihydro-4-oxo-7-(□-D-ribofuranosyl)-7H-pyrrolo[2,3-d]pyrimidin-5-carbonitrile

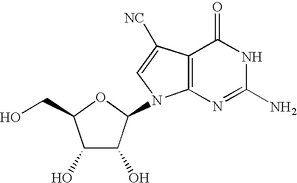

This compound was prepared following the procedures described in J. Am. Chem. Soc. 98: 7870 (1976).

### Example 9

2-Amino-5-ethyl-7-(□-D-ribofuranosyl)-7H-pyrrolo[2,3-d]pyrimidin-4(3H)-one

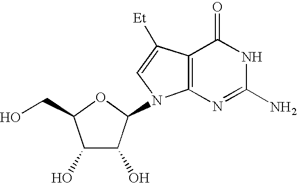

Step A: 2-Amino-7-(5-O-tert-butyldimethylsilyl-2,3-O-isopropylidene-□-D-ribofuranosyl)-4-chloro-5-ethyl-7H-pyrrolo[2,3-d]pyrimidine

To a stirred suspension of 2-amino-4-chloro-5-ethyl-1H-pyrrolo[2,3-d]pyrimidine [described in EP 866070 (1998)] (1.57 g, 8 mmol) in dry MeCN (48 mL) was added NaH (60% in mineral oil; 0.32 g, 8 mmol), and the mixture was stirred at room temperature for 1 h. A solution of 5-O-tert-butyldimethylsilyl-2,3-O-isopropylidene-□-D-ribofuranosyl chloride [generated in situ from the corresponding lactol (1.95 g, 6.4 mmol) according to Wilcox et al., Tetrahedron Lett., 27: 1011 (1986)] in dry THF (9.6 mL) was added at room temperature, and the mixture was stirred overnight, then evaporated to dryness. The residue was suspended in water (100 mL) and extracted with EtOAc (200+150 mL). The combined extracts were washed with brine, dried (Na₂SO₄) and evaporated. The residue was purified on a silica gel column using a solvent system of hexanes/EtOAc: 7/1. Appropriate fractions were collected and evaporated to dryness to give the title compound (1.4 g) as a colorless foam.

Step B: 2-Amino-4-chloro-5-ethyl-7-(□-D-ribofuranosyl)-7H-pyrrolo[2,3-d]pyrimidine

A mixture of the compound from Step A (1.19 g, 2.5 mmol) in MeOH (100 mL) and water (50 mL) was stirred with DOWEX H⁺ (to adjust pH of the mixture to 5) at room temperature for 2.5 h. The mixture was filtered and the resin thoroughly washed with MeOH. The combined filtrate and washings were evaporated and the residue coevaporated several times with water to yield the title compound (0.53 g) as a white solid.

Step C: 2-Amino-5-ethyl-7-(□-D-ribofuranosyl)-7H-pyrrolo[2,3-d]pyrimidin-4(3H)-one

A mixture of the compound from Step B (104 mg, 0.32 mmol) in 2N aqueous NaOH (10 mL) was stirred at reflux temperature for 15 min. The solution was cooled in ice bath, neutralized with 2 N aqueous HCl, and evaporated to dryness. The residue was suspended in MeOH, mixed with silica gel, and evaporated. The solid residue was placed onto a silica gel column (packed in a solvent mixture of CH₂Cl₂MeOH: 10/1) which was eluted with a solvent system of CH₂Cl₂/MeOH: 10/1 and 5/1. The fractions containing the product were collected and evaporated to dryness to yield the title compound (48 mg) as a white solid.

¹H NMR (CD₃OD): □ 1.22 (t, 3H), 2.69 (q, 2H), 3.69, 3.80 (2m, 2H), 4.00 (m, 1H), 4.22 (m, 1H), 4.45 (t, 1H), 5.86 (d, 1H, J=6.0 Hz), 6.60 (d, 1H, J=1.2 Hz).

### Example 10

2-Amino-7-(3-deoxy-□-D-ribofuranosyl)-7H-pyrrolo[2,3-d]pyrimidin-4(3H)-one

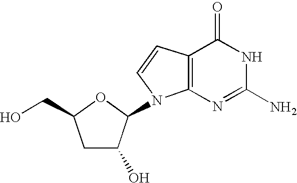

Step A: 2-Amino-7-(2,3-anhydro-□-D-ribofuranosyl)-4-methoxy-7H-pyrrolo[2,3-d]pyrimidine

To a mixture of 2-amino-7-(□-D-ribofuranosyl)-4-chloro-7H-pyrrolo[2,3-d]pyrimidine (1.8 g, 6.0 mmol) in acetonitrile (80 mL) were added a solution of H₂O/CH₃CN (1:9, 1.08 mL) and then α-acetoxyisobutyryl bromide (3.5 mL, 24 mmol). After 2 h stirring at room temperature, saturated aqueous NaHCO₃ (170 mL) was added and the mixture was extracted with EtOAc (300+200 mL). The combined organic phase was washed with brine (100 mL), dried (Na₂SO₄) and evaporated to a pale yellow foamy residue. This was suspended in anhydrous MeOH (80 mL) and stirred overnight with 25 mL of DOWEX OH⁻ resin (previously washed with anhydrous MeOH). The resin was filtered, washed thoroughly with MeOH and the combined filtrate evaporated to give a pale yellow foam (1.92 g).

Step B: 2-Amino-7-(3-deoxy-□-D-ribofuranosyl)-4-methoxy-7H-pyrrolo[2,3-d]pyrimidine

A solution of LiEt₃BH/THF (1M, 75 mL, 75 mmol) was added dropwise to a cold (ice bath) deoxygenated (Ar, 15 min) solution of the compound from Step A (1.92 g) under Ar. Stirring at 0° C. was continued for 4 h. At this point the reaction mixture was acidified with 5% aqueous acetic acid (110 mL), then purged with Ar for 1 h and and finally evaporated to a solid residue. Purification on a silica gel column using MeOH/CH₂Cl₂ as eluent yielded target compound as a colourless foam (1.01 g).

Step C: 2-Amino-7-(3-deoxy-□-D-ribofuranosyl)-7H-pyrrolo[2,3-d]pyrimidine-4(3H)-one

A mixture of compound from Step B (0.4 g, 1.4 mmol) in 2 N aqueous NaOH (40 mL) was stirred at reflux temperature for 3 h. The solution was cooled in ice bath, neutralized with 2 N aqueous HCl and evaporated to dryness. The residue was suspended in MeOH, mixed with silica and evaporated. The residue was placed onto a silica gel column which was eluted with CH₂Cl₂/MeOH: 10/1 and 5/1 to give the title compound as white solid (0.3 g).

¹H NMR (DMSO-d₆): δ1.85, 2.12 (2m, 2H), 3.55, 3.46 (2dd, 2H), 4.18 (m, 1H); 4.29 (m, 1H), 4.85 (7, 1H), 5.42 (d, 1H) 5.82 (d, 1H, J=2.4 Hz), 6.19 (s, 2H), 6.23 (d, 1H, J=3.6 Hz), 6.87 (d, 1H), 10.31 (s, 1H).

### Example 11

2-Amino-7-(2-O-methyl-□-D-ribofuranosyl)-7H-pyrrolo[2,3-d]pyrimidin-4(3H)-one

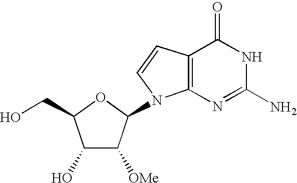

Step A: 2-Amino-4-chloro-7-(5-t-butyldimethylsilyl-2,3-O-isopropylidene-□-D-ribofuranosyl)-7H-pyrrolo[2,3-d]pyrimidine

HMPT (10.65 ml, 55 mmol) was added portionwise over 30 min. to a solution of 5-O-tert-butyldimethylsilyl-2,3-O-isopropylidene-D-ribofuranose (13.3 g, 44 mmol), dry THF (135 mL), CC1₄ (5.62 mL, 58 mmol) under N₂ at −76° C. After 30 min., the temp. was raised to −20° C. In a separate flask, a suspension of 2-amino-4-chloro-1H-pyrrolo-[2,3-d]-pyrimidine (15 g, 89 mmol) in CH₃CN (900 mL) was treated at 15° C. with 60% NaH (3.60 g., 90 mmol.). The reaction was stirred 30 min.whereupon the previous reaction mixture was cannulated with vigorous stirring. The reaction was stirred 16 hrs. and then concentrated in vacuo. The resulting semisolid was added to ice/water/EtOAc and extracted with EtOAc (3×200 mL), dried NaSO₄, filtered and evaporated. The resulting oil was chromatographed on silica gel (EtOAc/ Hexane 1/1) to afford the product as an oil (9.0 g).

Step B: 2-Amino4-chloro-7-(□-D-ribofuranosyl)-7H-pyrrolo[2,3-d]pyrimidine

A solution of the compound from Step A (5.76 g, 13 mmol) in MeOH/H₂O(1200 mL/600 mL) and Dowex WX8-400 (4.8 g) was stirred 16 hrs. at room temperature. The resin was filtered off and the filtrate evaporated to afford the title compound as a white solid; yield 3.47 g.

¹H NMR (DMSO-d₆): δ3.56 (m, 2H), 3.86 (m, 1H), 4.07 (m, 1H), 4.32 (m, 1H), 4.99 (t, 1H), 5.10 (d, 1H), 5.30 (d, 1H), 6.00 (d, 1H), 6.38 (d, 1H), 6.71 (s br, 2H), 7.39 (d, 1H).

Step C: 2-Amino-4-chloro-7-(2-O-methyl-□-D-ribofuranosyl)-7H-pyrrolo[2,3-d]pyrimidine

A solution of the compound from Step B (1.0 g, 3.3 mmol) in dry DMF (100 mL) at 15° C. was treated with 60% NaH (0.14 g, 3.5 mmol). After 30 min., iodomethane (47 g, 3.3 mmol) was added portionwise to the stirred solution. The reaction was stirred at room temperature for 16 hrs. and then evaporated at a temperature below 40° C. The resulting solid was chromatographed on silica gel to afford the product as a white solid; yield 0.81 g.

¹H NMR (DMSO-d₆): δ3.25 (s, 3H), 3.54 (m, 2H), 3.87 (m, 1H), 4.07 (m, 1H), 4.22 (m, 1H), 5.01 (m, 1H), 5.16 (d, 1H), 6.07 (d, 1H), 6.37 (d, 1H), 6.70 (s br, 2H), 7.40 (s, 1H). Mass spectrum: m/z 316 (M+1)⁺.

Step D: 2-Amino-7-(2-O-methyl-□-D-ribofuranosyl)-7H-pyrrolo[2,3-d]pyrimidin-4(3H)-one

A solution of the compound from Step C (80 mg, 0.25 mmol) in NaOH/H₂O (1.6 g/20 ml) was heated at reflux for 7 hrs., whereupon the solution was adjusted with dilute HCl to a pH of 7 and then evaporated. Chromatography of the resulting solid on silica gel with EtOAc/MeOH 8/2 afforded the product as a white solid; yield 64 mg.

¹H NMR (DMSO-d₆): δ3.25 (s, 3H), 3.52 (m, 2H) 3.81 (m, 1H), 4.00 (m, 1H), 4.19 (m, 1H), 5.10 (s br, 2H), 5.95 (d, 1H), 6.27 (d, 1H), 6.33 (s br, 2H), 6.95 (d, 1H), 10.55 (s br, 1H).

### Example 12

2-Amino-5-methyl-7-(□-D-ribofuranosyl)-7H-pyrrolo[2,3-d]pyrimidin4(3H)-one

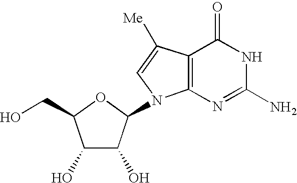

This compound is described in Biochemistry, 33: 2703 (1994) and was synthesized by the following procedure:

Step A: 2-Amino-7-(5-O-tert-butyldimethylsilyl-2,3-O-isopropylidene-□-D-ribofuranosyl)-4-chloro-5-methyl-7H-pyrrolo[2,3-d]pyrimidine

To a stirred suspension of 2-amino-4-chloro-5-methyl-1H-pyrrolo[2,3-d]pyrimidine (Liebigs Ann. Chem. 1984, 4, 708) (0.91 g, 5 mmol) in dry MeCN (30 ml) was added NaH (60% in mineral oil; 0.2 g, 5 mmol) and the mixture was stirred at room temperature for 0.5 h. A solution of 5-O-tert-butyldimethylsilyl-2,3-O-isopropylidene-α-D-ribofuranosyl chloride [generated in situ from the corresponding lactol (1.22 g, 4 mmol) according to Tetrahedron Lett. 27: 1011 (1986)] in dry THF (6 mL) was added at room temperature, and the mixture was stirred overnight, then evaporated to dryness. The residue was suspended in water (100 mL) and extracted with EtOAc (2×100 mL). The combined organic extracts were washed with brine, dried (Na₂SO₄) and evaporated. The residue was purified on a silica gel column using a solvent system of hexanes/EtOAc: 7/1 and 5/1. Appropriate fractions were collected and evaporated to dryness to give the title compound (0.7 g) as a colorless foam.

Step B: 2-Amino-4-chloro-5-methyl-7-(□-D-ribofuranosyl)-7H-pyrrolo[2,3-d]pyrimidine

A mixture of the intermediate from Step A (0.67 g, 1.4 mmol) in MeOH (70 ml) and water (35 ml) was stirred with DOWEX H⁺ (to adjust pH of the mixture to 5) at room temperature for 4 h. The mixture was filtered and the resin thoroughly washed with MeOH. The combined filtrate and washings were evaporated and the residue coevaporated several times with water to yield the title compound (0.37 g) as a white solid.

Step C: 2-Amino-5-methyl-7-(□-D-ribofuranosyl)-7H-pyrrolo[2,3-d]pyrimidin-4(3H)-one

A mixture of intermediate from Step B (100 mg, 0.32 mmol) in 2N aqueous NaOH (20 mL) was stirred at reflux temperature for 1.5 h. The solution was cooled in ice bath, neutralized with 2 N aqueous HCl and evaporated to dryness. The residue was suspended in MeOH, mixed with silica gel and evaporated. The solid residue was placed onto a silica gel column (packed in a solvent mixture of CH₂Cl₂/MeOH: 10/1) which was eluted with a solvent system of CH₂Cl₂/MeOH: 10/1 and 5/1. The fractions containing the product were collected and evaporated to dryness to yield the title compound (90 mg) as a white solid.

¹H NMR (DMSO-d₆): □2.15 (d, 3H), 3.47, 3.50 (2m, 2H), 3.75 (m, 1H), 3.97 (m, 1H), 4.17 (m, 1H), 4.89 (t, 1H), 4.96 (d, 1H), 5.14 (d, 1H), 5.80 (d, 1H, J=6.4 Hz), 6.14 (s, 2H), 6.60 (q, 1H, J=1.2 Hz), 10.23 (s, 1H).

### Example 13

2-Amino-3,4-dihydro-4-oxo-7-(2-O-methyl-□-D-ribofuranosyl)-7H-pyrrolo[2,3-d]pyrimidine-5-carbonitrile

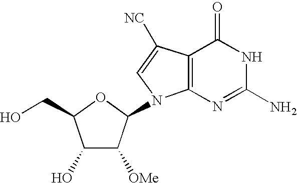

Step A: 2-Amino-4-chloro-7-□-D-ribofuranosyl-7H-pyrrolo[2,3-d]pyrimidine-5-carbonitrile

This intermediate was prepared according to J. Chem. Soc. Perkin Trans. 1. 2375 (1989).

Step B: 2-Amino-4-chloro-7-[3,5-O-(1,1,3,3-tetraisopropyldisiloxane-1,3-diyl)-□-D-ribofuranosyl]-7H-pyrrolo[2,3-d]pyrimidine-5-carbonitrile

To a solution of the compound from Step A (1.64 g, 5.00 mmol) in DMF (30 mL) was added imidazole (0.681 g, 10.0 mmol). The solution was cooled to 0° C. and 1,3-dichloro-1,1,3,3-tetraisopropyldisiloxane (1.58 g, 5.00 mmol) was added dropwise. The bath was removed and the solution stirred at room temperature for 30 minutes, evaporated in vacuo to an oil, taken up in ethyl acetate (150 mL) and washed with saturated aqueous sodium bicarbonate (50 mL) and with water (50 mL). The organic phase was dried over magnesium sulfate, filtered and evaporated in vacuo. The residue was purified on silica gel using ethyl acetate/hexane (1:2) as eluent. Fractions containing the product were pooled and evaporated in vacuo to give the desired product (2.05 g) as a colorless foam.

¹H NMR (DMSO-d₆): □1.03 (m, 28H), 3.92 (m, 1H), 4.01 (m, 1H), 4.12 (m, 1H), 4.24 (m, 2H), 5.67 (m, 1H), 5.89 (s, 1H), 7.17 (bs, 2H), 8.04 (s, 1H).

Step C: 2-Amino-4-chloro-7-[2-O-methyl-□-D-ribofuranosyl]-7H-pyrrolo[2,3-d]pyrimidine-5-carbonitrile

To a pre-cooled solution (0° C.) of the compound from Step B (1.70 g, 3.00 mmol) in DMF (30 mL) was added methyl iodide (426 mg, 3.00 mmol) and then NaH (60% in mineral oil) (120 mg, 3.00 mmol). The mixture was stirred at rt for 30 minutes and then poured into a stirred mixture of saturated aqueous ammonium chloride (100 mL) and ethyl acetate (100 mL). The organic phase was washed with water (100 mL), dried over magnesium sulfate, filtered and evaporated in vacuo. The resulting oily residue was co-evaporated three times from acetonitrile (10 mL), taken up in THF (50 mL) and tetrabutylammonium fluoride (1.1 mmol/g on silica) (4.45 g, 6.00 mmol) was added. The mixture was stirred for 30 minutes, filtered and the filtrate evaporated in vacuo. The crude product was purified on silica using methanol/dichloromethane (7:93) as eluent. Fractions containing the product were pooled and evaporated in vacuo to give the desired product (359 mg) as a colorless solid.

¹H NMR (DMSO-d₆): □3.30 (s, 3H), 3.56 (m, 2H) 3.91 (m, 1H), 4.08 (m, 1H), 4.23 (m, 1H), 5.11 (m, 1H), 5.23 (m, 1H), 7.06 (m, 1H), 7.16 (bs, 2H), 8.38 (s, 1H).

Step D: 2-Amino-3,4-dihydro-4-oxo-7-[2-O-methyl-□-D-ribofuranosyl]-7H-pyrrolo[2,3-d]pyrimidine-5-carbonitrile

To a solution of the compound from Step D in DMF (5.0 mL) and dioxane (3.5 mL) was added syn-pyridinealdoxime (336 mg, 2.75 mmol) and then tetramethylguanidine (288 mg, 2.50 mmol). The resulting solution was stirred overnight at rt, evaporated in vacuo and and co-evaporated three times from acetonitrile (20 mL). The oily residue was purified on silica gel using methanol/dichloromethane (7:93) as eluent. Fractions containing the product were pooled and evaporated in vacuo to give the desired product (103 mg) as a colorless solid.

¹H NMR (DMSO-d₆): □3.30 (s, 3H), 3.57 (m, 2H), 3.86 (m, 1H), 4.00 (m, 1H), 4.21 (m, 1H), 5.07 (m, 1H), 5.17 (m, 1H), 5.94 (m, 1H), 6.56 (bs, 2H), 7.93 (s, 1H), 10.82 (bs, 1H).

### Example 14

2-Amino-5-methyl-7-(2-O□methyl-□-D-ribofuranosyl)-7H-pyrrolo[2,3-d]pyrimidin-4(3H)-one

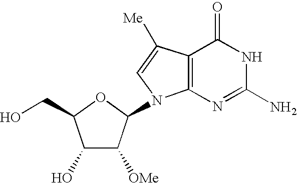

Step A: 2-Amino-4-chloro-5-methyl-7-(2-O-methyl-□-D-ribofuranosyl)-7H-pyrrolo[2,3-d]-pyrimidine

Into a solution of the compound from Example 12, Step B (188 mg, 0.6 mmol) in anhydrous DMF (6 mL) was added NaH (60% in mineral oil; 26 mg, 0.66 mmol). The mixture was stirred at room temperature for 0.5 h and then cooled. MeI (45 □L) was added at 0° C. and the reaction mixture allowed to warm to 15° C. in 5 h. Then the mixture was poured into ice-water (20 mL) and extracted with CH₂Cl₂ (100+50 mL). The combined organic extracts were washed with water (50 mL), brine (50 mL) and dried (Na₂SO₄). The evaporated residue was purified on a silica gel column with a solvent system of CH₂Cl₂/MeOH: 30/1. Appropriate fractions were pooled and evaporated to yield the title compound (50 mg) as a colorless glass.

Step B: 2-Amino-7-(2-O-methyl-□-D-ribofuranosyl)-5-methyl-7H-pyrrolo[2,3-d]pyrimidin-4(3H)-one

A solution of the compound from Step A (50 mg, 0.15 mmol) in 0.5M NaOMe/MeOH (4 mL) was stirred at reflux temperature for 1.5 h. The mixture was cooled, mixed with silica gel and evaporated to dryness. The silica gel was loaded onto a silica gel column and eluted with a solvent system of CH₂Cl₂/MeOH: 30/1. The fractions containing the product were collected and evaporated to yield 2-amino-7-(2-O-methyl-□-D-ribofuranosyl)-4-methoxy-5-methyl-7H-pyrrolo[2,3-d]pyrimidine (40 mg). This was mixed with 2 N aqueous NaOH (4 mL) and stirred at reflux temperature for 10 h. The mixture was cooled in ice bath, neutralized with 2 N aqueous HCl and evaporated. The solid residue was suspended in MeOH, mixed with silica gel and evaporated. The silica gel was loaded onto a silica gel column and eluted with a solvent system of CH₂Cl₂/MeOH: 5/1. Appropriate fractions were pooled and evaporated to give the title compound (40 mg) as a white solid.

¹H NMR (DMSO-d₆): □2.18 (s, 3H), 3.26 (s, 3H), 3.45, 3.52 (2m, 2H), 3.82 (m, 1H), 3.97 (dd, 1H), 4.20 (m, 1H), 4.99 ((t, 1H), 5.10 (d, 1H), 5.94 (d, 1H, J=7.0 Hz), 6.19 (bs, 2H), 6.68 (s, 1H), 10.60 (br, 1H).

### Example 15

2-Amino-7-(2-deoxy-2-fluoro-β-D-arabinofuranosyl)-7H-pyrrolo[2,3-d]pyrimidin-4(3H)-one

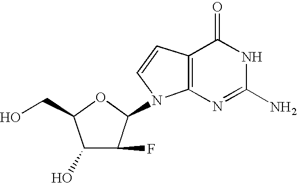

This compound was prepared following the procedures described in J. Med. Chem. 38: 3957 (1995).

### Example 16

2-Amino-7-(□-D-arabinofuranosyl)-7H-pyrrolo[2,3-d]pyrimidin-4(3H)-one

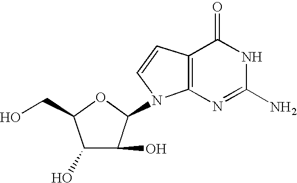

This compound was prepared following the procedures described in J. Org. Chem. 47: 226 (1982).

### Example17

2-Amino-7-(□-D-arabinofuranosyl)-3,4-dihydro-4-oxo-7H-pyrrolo[2,3-d]pyrimidine-5-carbonitrile

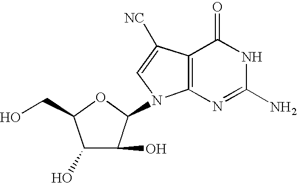

Step A: 2-Amino-7-(□-D-arabinofuranosyl)4-chloro-7H-pyrrolo[2,3-d]pyrimidine-5-carbonitrile

This intermediate was prepared according to J. Chem. Soc. Perkin Trans. 1, 2375 (1989).

Step B: 2-Amino-7-(□-D-arabinofuranosyl)-3,4-dihydro-4-oxo-7H-pyrrolo[2,3-d]pyrimidine-5-carbonitrile

To a solution of the compound from Step A (163 mg, 0.50 mmol) in DMF (5.0 mL) and dioxane (3.5 mL) was added syn-pyridinealdoxime (336 mg, 2.75 mmol) and then tetramethylguanidine (288 mg, 2.50 mmol). The resulting solution was stirred overnight at rt, evaporated in vacuo and and co-evaporated three times from acetonitrile (20 mL). The oily residue was purified on silica using methanol/dichloromethane (1:4) as eluent. Fractions containing the product were pooled and evaporated in vacuo to give the desired product (72 mg) as a colorless solid.

¹H NMR (DMSO-d₆): □3.60 (m, 2H), 3.73 (m, 1H), 4.01 (m, 2H), 5.06 (m, 1H), 5.48 (m, 2H), 6.12 (m, 1H), 6.52 (bs, 2H), 7.70 (s, 1H), 10.75 (bs, 1H).

### Example 18

2-Amino-5-methyl-7-(□-D-arabinofuranosyl)-7H-pyrrolo[2,3-d]pyrimidin-4(3H)-one

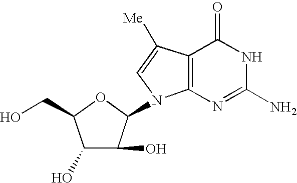

Step A 2-Amino-7-(2,3,5-tri-O-benzyl-□-D-arabinofuranosyl)-4-chloro-5-methyl-7H-pyrrolo[2,3-d]pyrimidine

To a solution of 1-O-p-nitrobenzyl-D-arabinofuranose (3.81 g, 6.70 mmol) in DCM was bubbled HBr until TLC (hexane/ethylacetate (2: 1)) showed complete reaction (about 30 min). The reation mixture was filtered and evaporated in vacuo. The oily residue was taken up in acetonitrile (10 mL) and added to a vigorously stirred suspension of 2-amino4-chloro-5-methyl-7H-pyrrolo[2,3-d]pyrimidine (Liebigs Ann. Chem. (1984), 4, 708) (1.11 g, 6.00 mmol) KOH (1.12 g, 20.0 mmol) and tris[2-(2-methoxyethoxy)ethyl]amine (0.216 g, 0.67 mmol) in acetonitrile (80 mL). The resulting suspension was stirred at rt for 30 min, filtered and evaporated in vacuo. The crude product was purified on silica using hexane/ethylacetate (3:1) as the eluent. Fractions containing the product were pooled and evaporated in vacuo to give the desired product (1.13 g) as a colorless foam.

Step B: 2-Amino-7-□-D-arabinofuranosyl-4-chloro-5-methyl-7H-pyrrolo[2,3-d]pyrimidine

To a precooled (−78° C.) solution of the compound from Step A (0.99 g, 1.7 mmol) in dichloromethane (30 mL) was added borontrichloride (IM in dichloromethane) (17 mL, 17.0 mmol) over a 10 min. The resulting solution was stirred at −78° C. for lh, allowed to warm to −15° C. and stirred for another 3h. The reaction was quenched by addition of methanol/dichloromethane (1:1) (15 mL), stirred at −15° C. for 30 min, and pH adjusted to 7.0 by addtion of NH₄OH. The mixture was evaporated in vacuo and the resulting oil purified on silica using methanol/dichloromehane (1:9) as eluent. Fractions containing the product were pooled and evaporated in vacuo to give the desired product (257 mg) as a colorless foam.

Step C:2-Amino-7-(□-D-arabinofuranosyl)-5-methyl-7H-pyrrolo[2,3-d]pyrimidin-4(3H)-one

To the compound from Step B (157 mg, 0.50 mmol) was added NaOH (2M, aqueous) (2 mL). The resulting solution was stirred at relux for 1h, cooled and neutralized by addition of HCl (2M, aqueous). The mixture was evaporated in vacuo and the crude product purified on silica using methanol/dichloromehane (2:8) as eluent. Fractions containing the product were pooled and evaporated in vacuo to give the desired product (53 mg) as a colorless powder.

¹H NMR (DMSO-d₆): □2.13 (d, 3H), 3.58 (m, 2H), 3.71 (m, 1H), 4.00 (m, 2H), 5.09 (m, 1H), 6.22 (bs, 2H), 5.50 (m, 2H), 6.12 (m, 1H), 6.64 (s, 1H), 10.75 (bs, 1H).

### Example 19

2-Amino-7-(3-deoxy-3-fluoro-□-D-ribofuranosyl)-7H-pyrrolo[2,3-d]pyrimidin-4(3H)-one

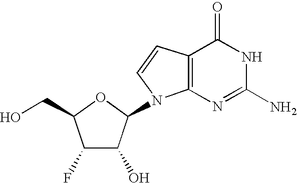

A solution 1-O-acetyl-2-O-benzyl-5-O-(p-toluoyl)-3-deoxy-3-fluoro-D-ribofuranose (410 mg, 1.01 mmol) (prepared by a modified method described for similar sugar derivatives, Helv. Chim. Acta 82: 2052 (1999) and J. Med. Chem. 1991, 34, 2195) in anhydrous CH₂Cl₂ (1.5 mL) was cooled to −15° C. in a dry ice/CH₃CN bath. After cooling the reaction mixture for 10 min. under the argon atmosphere, 33% HBr/AcOH (370 □L, 1.5 equiv.) was added slowly over 20 min keeping the bath temperature around −15° C. After the addition was complete, the reaction mixture was stirred at −10° C. for 1 hr. The solvent was removed under reduced pressure and the residue azeotroped with anhydrous toluene (5×10 mL). In a separate flask, 2-amino-4-chloro-7H-pyrrolo[2,3-d]pyrimidine (210 mg, 1.2 mmol) was suspended in anhydrous CH₃CN (10 mL) and cooled to −10° C. To this was added 60% NaH dispersion in oil (57 mg) in two portions, and the reaction mixture was stirred for 45 min. during which time the solid dissolved and the bath temperature rose to 0° C. The bath was removed and stirring was continued for about 20 additional min. It was cooled back to −10° C. and the bromo sugar, prepared above, was taken up in anhydrous CH₃CN (1.5 mL) and added slowly to the anion of nucleobase. After the addition was complete, the reaction mixture was stirred for an additional 45 min allowing the temperature of the reaction to rise to 0° C. The bath was removed and the reaction allowed to stir at room temperature for 3 hr. Methanol was added carefully to the reaction mixture and the separated solid removed by filtration. The solvent was removed under reduced pressure and the residual oil dissolved in EtOAc (50 mL) and washed with water (3×20mL). The organic layer was dried over Na₂SO₄ and concentrated to give an oil. It was purified by column chromatography to furnish fully protected 2-amino-7-(5-O-(p-toluoyl)-2-O-benzyl-3-deoxy-3-fluoro-□-D-ribofuranosyl)-4-chloro-7H-pyrrolo[2,3-d]pyrimidine (190 mg) as an □/□ mixture (1:1). After conversion of 4-chloro to 4-oxo by heating the compound with 2N NaOH/dioxane mixture at 105° C. and after the usual workup the residue was debenzylated using 20 mol % w/w of 10% Pd/C and ammonium formate in refluxing methanol to give title compound after purification by HPLC; yield 10%. ESMS: calcd. for C₁₁H₁₃FN₄O₄284.24, found 283.0 (M+1).

### Example 20

2-Amino-3,4-dihydro-4-oxo-7-(2-deoxy-□-D-ribofuranosyl)-7H-pyrrolo[2,3-d]pyrimidine-5-carbonitrile

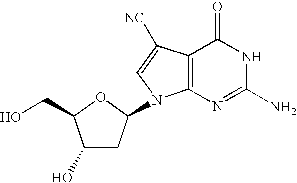

This compound was prepared following the procedures described in Synthesis 1327 (1998).

### Example 21

6-Amino-1-(□-D-ribofuranosyl)-1H-imidazo[4,5-c]pyridin-4(5H)-one

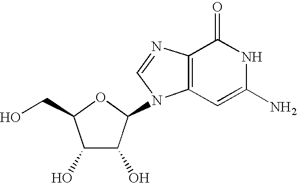

This compound was prepared following the conditions described in J. Am. Chem. Soc. 97: 2916 (1975).

### Example 22

2-Amino-7-(2-O-methyl-□-D-ribofuranosyl)-5H-pyrrolo[3,2-d]pyrimidin-4-(3H)-one

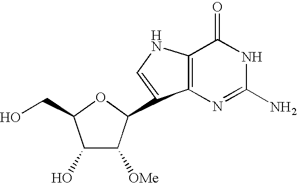

To a suspension of 2-amino-5H-pyrrolo[3,2-d]pyrimidin-4(3H)-one (9-deazaguanine) (0.454 g, 3.0 mmol) (prepared according to J. Org. Chem.1978, 43, 2536) and 2-O-methyl-1,3,5-tri-O-benzoyl-□-D-ribofuranose (1.54 g, 3.2 mmol) in dry nitromethane (23 mL) at 60° C. was added stannic chloride (0.54 mL, 4.5 mmol). The reaction mixture was maintained at this temperature for 0.5 hr., cooled and poured onto ice-cold saturated sodium bicarbonate solution (70 mL). The insoluble material was filtered through florisil and washed with ethyl acetate (3×50 mL). The filtrate was extracted with ethyl acetate (2×50 mL), and organic layer was washed with water (2×50 mL), dried over Na₂SO₄ and evaporated to dryness. Chromatography of the resulting foam on silica gel with CH₂Cl₂/MeOH(14:1) afforded the benzoylated product (0.419 g, 30% yield). To a suspension of the benzoylated product (0.25 g) in MeOH (2.4 mL) was added t-butylamine (0.52 mL) and stirring at room temperature was continued for 24 hrs. followed by addition of more t-butylamine (0.2 mL). The reaction mixture was stirred at ambient temperature overnight, concentrated in vacuum and the residue was purified by flash chromatography over silica gel using CH₂Cl₂/MeOH (85:15) as eluent giving the desired compound as a foam (0.80 g).

¹H NMR (200 MHz, DMSO-d₆): □Hz3.28 (s, 3H), 3.40-3.52 (m, 3H), 3.87-3.90 (m, 1H), 4.08-4.09 (m, 1H), 4.67 (d, 1H, J=5.2 Hz), 4.74 (d, 1H, J=7.0 Hz), 5.62 and 5.50 (2 bs, 3H), 7.14 (d, 1H, J=2.6 Hz), 10.43 (s,1H), 11.38 (s,1H); Mass spectrum: calcd. for C₁₂H₁₆N₄O₅: 296.28; found: 295.11.

### Example 23

6-Amino-1-(3-deoxy-□-D-ribofuranosyl)-1H-imidazo[4,5-c]pyridine-4(5H)-one (3′-deoxy-3-deaza-guanosine)

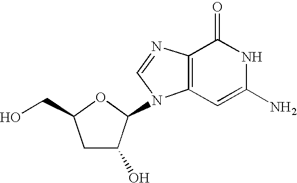

Step A: 3-Deoxy-4-O-p-toluoyl-2-O-acetyl-□-D-ribofuranosyl acetate

A solution of 3-deoxy-4-O-p-toluoyl-1,2-O-isopropylidene-□-D-ribofuranose (Nucleosides Nucleotides 1994, 13, 1425 and Nucleosides Nucleotides 1992, 11, 787) (5.85 g, 20 mmol) in 64 mL of 80% acetic acid was stirred at 85° C. overnight. The reaction mixture was concentrated and co-evaporated with toluene. The residue was dissolved in 90 mL of pyridine. Acetic anhydride (6 mL) was added at 0° C., and the reaction mixture was stirred at rt for 6 h. After condensation, the residue was dissolved in ethyl acetate and washed with aqueous sodium bicarbonate solution, water and brine. The organic phase was dried and concentrated. Chromatographic purification on a silica gel column using 3:1 and 2:1 hexanes-EtOAc as eluent provided 5.51 g of the title compound as a clear oil.

¹H NMR (CDCl₃): □□1.98 (s, 3H), 2.09 (s, 3H), 2.15-2.35 (m, 2H), 2.41 (s, 3H) 4.27-4.42 (m, 1H), 4.46-4.58 (m, 1H), 4.65-4.80 (m, 1H), 5.21-5.28 (m, 1H), 6.20 (s, 1H), 7.19-7.31 (m, 2H), 7.90-8.01 (m, 2H).

Step B: Methyl 5-cyanomethyl-1-(3-deoxy-4-O-p-toluoyl-2-O-acetyl-□-D- ribofuranosyl)-1H-imidazole-4-carboxylate

A mixture of methyl 5(4)-(cyanomethyl)-1H-imidazole-4(5)-carboxylate (J. Am. Chem. Soc.1976, 98, 1492 and J. Org. Chem. 1963, 28, 3041) (1.41 g, 8.53 mmol), 1,1,1,3,3,3-hexamethyldisilazane (20.5 mL) and ammonium sulfate (41 mg) was refluxed at 125° C. under Ar atmosphere for 18 h. After evaporation, the residue was dissolved in 10 mL of dichloroethane. A solution of the compound from Step A (2.86 g, 8.5 mmol) in 10 mL of dichloroethane was added followed by addition of SnCl₄ (1.44 mL, 3.20 g). The resulted reaction mixture was stirred at rt overnight and diluted with chloroform. The mixture was washed with aqueous sodium bicarbonate, water and brine. The organic phase was dried and concentrated. Chromatographic purification of the residue on a silica gel column using 1:1, 1:2, and 1:3 hexanes-EtOAc as eluent provided 2.06 g of the title compound as a white foam.

¹H NMR (CDCl₃) □2.15 (s, 3H), 2.28-2.40 (m, 2H), 2.38 (s, 3H), 3,87 (s, 3H), 4.46 (dd, 2H, J=7.6, 2.0 Hz), 4.50-4.57 (m, 1H), 4.68-4.75 (m, 1H), 4.76-4.83 (m, 1H), 5.41 (d, 1H, J=5.6 Hz), 5.91 (s, 1H), 7.24-7.28 (m, 2H), 7.80 (s, 1H), 7.82-7.90 (m, 2H); ¹³C NMR (CDCl₃) □13.1, 20.7, 21.6, 31.5, 51.8, 63.5, 77.9, 79.2, 89.8, 115.1, 126.2, 129.3, 129.5, 131.7, 135.1, 144.3, 163.1, 166.1, 170.3.

Step C: 6-Amino-1-(3-deoxy-□-D-ribofuranosyl)-1H-imidazo[4,5-c]pyridine-4(5H)-one

A solution of the compound from Step B (2.00 g, 4.53 mmol) in methanol (30 mL) was saturated with ammonia at 0° C. Concentrated ammonium hydroxide (30 mL) was added and the sealed metal reactor was heated at 85° C. for 5 h. After cooling to rt, the reaction mixture was transferred directly onto a silica gel column. Elution with 4:1, 3:1 and 2:1 CHCl₃-MeOH provided 0.79 g of the title compound as a white solid.

¹H NMR (DMSO-d₆): □□2.41-2.46 (m, 1H), 2.52-2.58 (m, 1H), 3.48-3.55 (m, 1H), 3.60-3.70 (m, 1H), 4.27-4.36 (m, 2H), 4.97 (t, 1H, J=5.6 Hz), 5.44 (s, 1H), 5.47 (s, 1H), 5.60 (s, 2H), 5.66, (d, 1H, J=4.4 Hz), 7.90 (s, 1H), 10.33 (s, 1H); ¹³C NMR (DMSO d₆) □34.1, 62.4, 70.4, 74.7, 80.4, 91.6, 123.0, 136.3, 141.9, 147.6, 156.5.

### Example 24

6-Amino-1-(3-deoxy-3-fluoro-□-D-ribofuranosyl)-1H-imidazo[4,5-c]pyridin-4(3H)-one

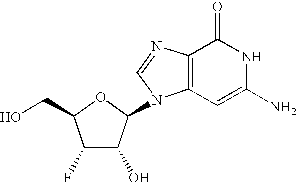

This compound was prepared in a manner similar to the preparation of 2-amino-7-(3-deoxy-3-fluoro-□-D-ribofuranosyl)-7H-pyrrolo[2,3-d]pyrimidin-4(3H)-one (Example 23).

### Example 25

1-(□-D-Ribofuranosyl)-1H-pyrazolo[3,4-d]pyrimidin-4(3H)-one (Allopurinol riboside)

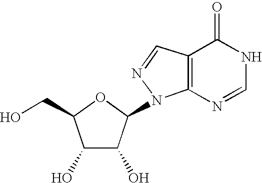

This compound was obtained from commercial sources.

### Example 26

9-(β-D-Arabinofuranosyl)-9H-purin-6(1H)-one

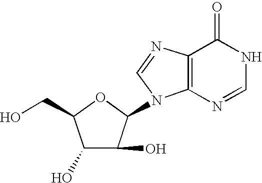

This compound was prepared following the conditions described in J. Med. Chem. 18: 721(1975).

### Example 27

2-Amino-7-(2-O-methyl-β-D-ribofuranosyl)-7H-pyrrolo[2,3-d]pyrimidin-4(3H)-thione

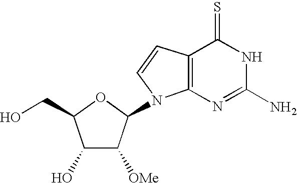

A solution of the compound from Example 11, Step C (1.5 g, 5 mmol), thiourea (0.4 g, 5.2 mmol.) in abs. EtOH was refluxed for 16 hrs. The solution was evaporated and the resulting oil chromatographed on silica gel (EtOAc/MeOH: 9/1) to afford the desired product as a foam.

¹H NMR (DMSO-d₆): □3.30 (s, 3H), 5.00-5.06 (t, 1H), 5.19 (d, 1H), 5.95 (d, 1H), 6.43 (d, 1H), (d, 1H).

### Example 28

2-Amino-7-(□-D-ribofuranosyl)-7H-pyrrolo[2,3-d]pyrimidine

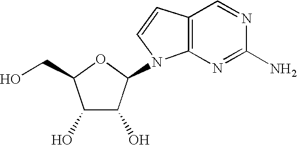

This compound was obtained from commercial sources.

### Example 29

2-Amino-4-chloro-7-(2-O-methyl-□-D-ribofuranosyl)-7H-pyrrolo[2,3-d]pyrimidin-5-carbonitrile

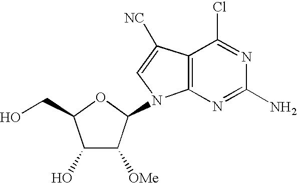

This compound was prepared as described in Example 13, Steps A-C.

### Example 30

2-Amino-4-chloro-5-ethyl-7-(2-O-methyl-□-D-ribofuranosyl)-7H-pyrrolo[2,3-d]pyrimidine

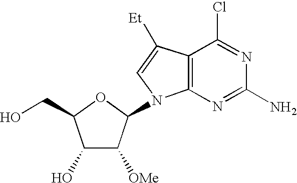

Step A: 2-Amino-4-chloro-5-ethyl-7-[3,5-O-(tetraisopropyldisiloxane-1,3-diyl)-□-D-ribofuranosyl]-7H-pyrrolo[2,3-d]pyrimidine

To a solution of 2-amino-4-chloro-5-ethyl-7-(□-D-ribofuranosyl)-7H-pyrrolo[2,3-d]pyrimidine (0.300 g, 0.913 mmol) in pyridine (8 mL) was added 1,3-dichloro-1,1,3,3-tetraisopropyldisiloxane (0.317 g, 1.003 mmol) dropwise. The solution stirred at rt overnight, evaporated in vacuo to an oil, and evaporated repeatedly from acetonitrile. The crude product was purified on silica using 5% methanol in dichloromethane as eluent. Fractions containing the product were pooled and evaporated in vacuo to give the desired product (254 mg) as a colorless solid.

Step B: 2-Amino-4-chloro-5-ethyl-7-(2-O-methyl-□-D-ribofuranosyl)-7H-pyrrolo[2,3-d]pyrimidine

To a pre-cooled solution (0° C.) of the compound from step A (192 mg, 0.337 mmol) in DMF (3 mL) was added methyl iodide (45.4 mg, 0.320 mmol) and ) then NaH (60% in mineral oil) (8.10 mg, 0.320 mmol). The mixture was stirred at rt for 45 minutes and then poured into a stirred mixture of saturated aqueous ammonium chloride (10 mL) and ethyl acetate (10 mL). The organic phase phase was washed with brine (10 mL) and dried over MgS₄O and evaporated in vacuo. The resulting oily residue was taken up in THF (5 mL) and tetrabutylammonium fluoride (1.1 mmol/g on silica) (0.529 g, 0.582 mmol) was added. The mixture was stirred for 30 minutes, filtered and the filtrate evaporated in vacuo. The crude product was purified on silica using 10% methanol in dichloromethane as eluent. Fractions containing the product were pooled and evaporated in vacuo to give the desired product (66 mg) as a colorless solid.

¹H NMR (DMSO-d₆): □1.15 (t, 3H), 2.65 (q, 2H), 3.20 (s, 3H), 3.51 (m, 2H), 3.84 (m, 1H), 4.04 (m, 1H), 4.21 (m, 1H), 4.99 (m, 2H), 5.15 (m, 2H), 6.07 (m, 2H), 6.62 (s br, 2H), 7.06 (s, 2H).

### Example 31

2-Amino-4-chloro-5-methyl-7-(2-O-methyl-□-D-ribofuranosyl)-7H-pyrrolo[2,3-d]pyrimidine

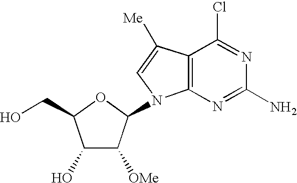

This compound was prepared as described in Example 14, Step A.

¹H NMR (CD₃OD): □2.33 (s, 3H), 3.39 (s, 1H), 3.72, 3.83 (2dd, 2H), 4.03 (m, 1H), 4.17 (t, 1H), 4.39 (dd, 1H), 5.98 (d, 1H, J=5.9 Hz), 6.7 (bs, 2H), 7.01 (s, 1H).

### Example 32

2-Amino-4-chloro-7-(2-O-methyl-□-D-ribofuranosyl)-7H-pyrrolo[2,3-d]pyrimidine

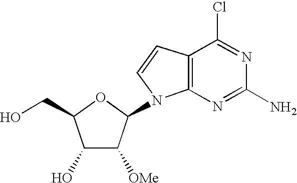

This compound was synthesized as described in Example 11, Steps A-C.

### Example 33

2-Amino-4-chloro-7-(□-D-ribofuranosyl)-7H-pyrrolo[2,3-d]pyrimidine

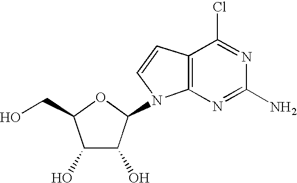

This compound was prepared following the procedures described in Helv. Chim. Acta 73: 1879 (1990).

### Example 34

2-Amino-4-chloro-5-methyl-7-(□-D-ribofuranosyl)-7H-pyrrolo[2,3-d]pyrimidine

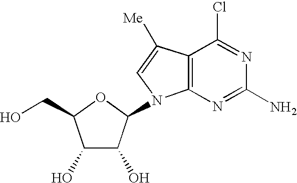

The compound was prepared as described in Example 12, Steps A-B.

¹H NMR (DMSO-d₆): □2.29 (s, 3H), 3.54 (m, 2H), 3.84 (m, 1H), 4.04 (dd, 1H, J₁=3.0, J₂=4.9 Hz), 4.80-5.50 (bs, 3H), 4.28 (t, 1H), 5.98 (d, 1H, J=6.5 Hz), 6.7 (bs, 2H), 7.13 (s, 1H).

### Example 35

2-Amino-4-chloro-5-ethyl-7-(β-D-ribofuranosyl)-7H-pyrrolo[2,3-d]pyrimidine

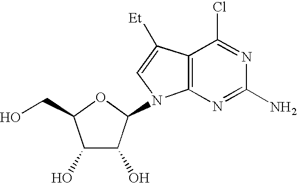

This compound was prepared as described in Example 9, Steps A-B.

¹H NMR (DMSO-d₆): δ2.00 (t, 3H), 2.69 (q, 2H), 3.48 (dd, 1H, J₁=4.2 Hz, J₂=11.8 Hz), 3.56 (dd, 1H, J₁=4.3 Hz, J₂ =11.8 Hz), 3.80 (m, 1H), 4.02 (dd, 1H, J₁=3.1 Hz, J₂=5.0 Hz), 4.62 (t, 1H), 5.0 (bs, 2H), 5.2 (bs, 1H), 5.60 (d, 1H, J=6.4 Hz), 6.61 (bs, 2H), 7.09 (s, 1H).

### Example 36

2-Amino-6-chloro-9-(□-D-ribofuranosyl)-9H-purine

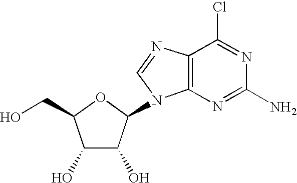

This compound was obtained from commercial sources.

### Example 37

2-Amino-4-chloro-7-(□-D-ribofuranosyl)-7H-pyrrolo[2,3-d]pyrimidine-5-carbonitrile

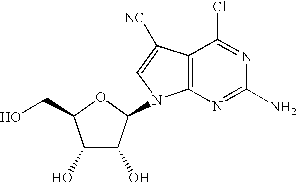

This compound was prepared following the procedures described in J. Chem. Soc. Perkin Trans. 1, 2375 (1989).

### Example 38

2-Amino-4-chloro-7-(2-deoxy-2-fluoro-β-D-arabinofuranosyl)-7H-pyrrolo[2,3-d]pyrimidine

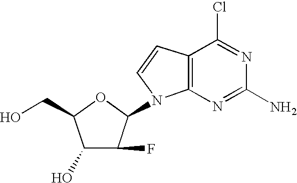

This compound was prepared following the procedures described in J. Med. Chem. 38: 3957 (1995).

### Example 39

2-Amino-4-chloro-5-methyl-7-(□-D-arabinofuranosyl)-7H-pyrrolo[2,3-d]pyrimidine

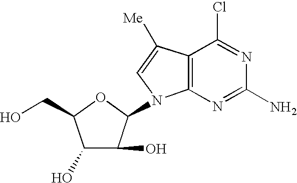

The compound was prepared as described in Example 18, Steps A-B. ¹H NMR (DMSO-d₆): □2.24 (s, 3H), 3.60 (m, 3H), 3.98 (m, 2H), 4.98 (m, 1H), 5.43 (bs, 2H), 6.25 (s, 1H), 6.57 (bs, 2H), 7.01 (s, 1H).

### Example 40

2′-O-Methylcytidine

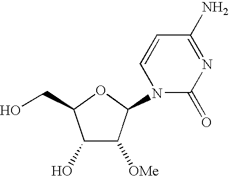

This compound was obtained from commercial sources.

### Example 41

3′-Deoxy-3′-methylcytidine

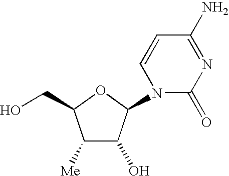

This compound was prepared following the procedures described in U.S. Pat. No. 3,654,262 (1972), which is incorporated by reference herein in its entirety.

### Example 42

3′-Deoxycytidine

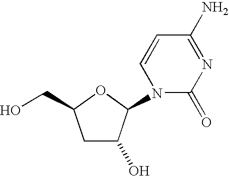

This compound was obtained from commercial sources.

### Example 43

3′-Deoxy-3′-fluorocytidine

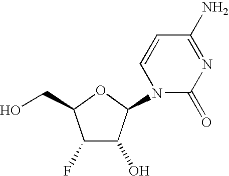

This compound was prepared following the procedures described in J. Med. Chem. 34: 2195 (1991).

### Example 44

1-(β-D-Arabinofuranosyl)-1H-cytosine

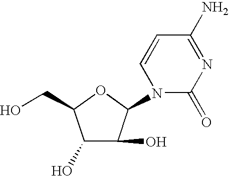

This compound was obtained from commercial sources.

### Example 45

2′-Amino-2′-deoxycytidine

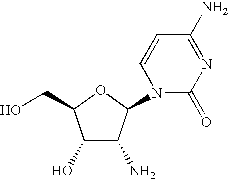

This compound was obtained from commercial sources.

### Example 46

3′-Deoxy-3′-methyluridine

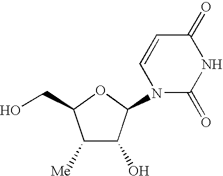

This compound was prepared following procedures described in U.S. Pat. No. 3,654,262, which is incorporated by reference herein in its entirety.

### Example 47

3′-Deoxy-3′-fluorouridine

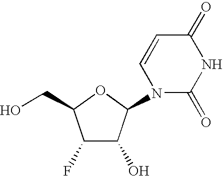

This compound was prepared following procedures described in J. Med. Chem. 34: 2195 (1991) and FEBS Lett. 250: 139 (1989).

### Example 48

3′-Deoxy-5-methyluridine

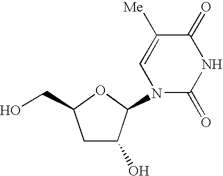

This compound was obtained from commercial sources.

### Example 49

3′-Deoxy-2′-O-(2-methoxyethyl)-3′-methyl-5-methyluridine

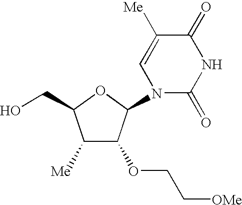

Step A: 5′-O-(tert-butyldiphenylsilyl)-3′-O-(3-tert-butylphenoxythiocarbonyl)-2′-O-(2-methoxyethyl)-5-methyluridine

This compound was synthesized by the reaction of the corresponding 5′-protected-2′-substituted-5-methyluridine with 3′-t-butylphenoxy chlorothionoformate following the similar procedure for the preparation of 3′-phenoxythiocarbonyl-2′-deoxy derivative (Synthesis 1994, 1163).

Step B: 5′-O-(tert-Butyldiphenylsilyl)-3′-deoxy-2′-O-(2-methoxyethyl)-3′-(2-phenylethenyl)-5-methyluridine

To a solution of 5′-O-(tert-butyldiphenylsilyl)-3′-O-(3-tert-butylphenoxythiocarbonyl)-2′-O-(2-methoxyethyl)-5-methyluridine (15.0 g, 20.0 mmol) in 150 mL of benzene was added PhCH═CHSnBu₃ (18.7 g, 50 mmol). The resulting solution was degassed three times with argon at rt and 45° C. After AIBN (1.0 g, 6.1 mmol) was added, the resulting solution was refluxed for 2 h. Another portion of AIBN (1.0 g, 6.1 mmol) was added after cooling to about 40° C. and refluxed for 2 h. This procedure was repeated until the starting material disappeared. The solvent was evaporated and the residue was purified by flash chromatography on a silica gel column using 10:1 and 5:1 hexanes-EtOAc as eluent to give 1.74 g of 5′-O-(tert-butyldiphenylsilyl)-3′-deoxy-2′-O-(2-methoxyethyl)-3′-(2-phenylethenyl)5-methyluridine as a white foam.

¹H NMR (CDCl₃): δ1.13, (s, 9H), 1.43 (s, 3H), 3.18-3.30 (m, 1H), 3.37 (s, 3H), 3.58-3.62 (m, 2H), 3.79-3.80 (m, 2H), 4.06-4.37 (m, 4H), 4.95 (s, 1H), 6.25-6.40 (m, 1H), 6.62 (d, 1H, J=16 Hz), 7.27-7.71 (m, 16H), 9.21 (s, 1H); ¹³C NMR (CDCl₃) δ11.9, 19.6, 27.2, 45.3, 59.0, 62.1, 70.2, 72.0, 84.6, 87.1, 90.2, 110.4, 122.8, 126.4, 127.8, 128.0, 128.3, 128.6, 130.0, 132.7, 133.5, 134.7, 135.3, 135.4, 136.9, 150.3, 154.1; HRMS (FAB) m/z 641.302 (M+H)⁺(C₃₇H₄₅N₂O₆Si requires 641.304).

Step C: 5′-O-(tert-Butyldiphenylsilyl)-3′-deoxy-3′-(hydroxymethyl)-2′-O-(2-methoxyethyl)-5-methyluridine

To a solution of 5′-O-(tert-butyldiphenylsilyl)-3′-deoxy-2′-O-(2-methoxyethyl)-3′-(2-phenylethenyl)-5-methyluridine. (5.0 g, 7.8 mmol) and N-methylmorpholine N-oxide (NMO) (1.47 g, 12.5 mmol) in 150 mL of dioxane was added a catalytic amount of osmium tetraoxide (4% aqueous solution, 2.12 mL, 85 mg, 0.33 mmol). The flask was covered by aluminum foil and the reaction mixture was stirred at rt overnight. A solution of NaIO₄ (5.35 g, 25 mmol) in 5 mL of water was added to the above stirred reaction mixture. The resulting reaction mixture was stirred for 1 h at 0° C. and 2 h at rt, followed by addition of 10 mL of ethyl acetate. The mixture was filtered through a celite pad and washed with ethyl acetate. The filtrate was washed 3 times with 10% aqueous Na₂S₂O₃ solution until the color of aqueous phase disappeared. The organic phase was further washed with water and brine, dried (Na₂SO₄) and concentrated. The aldehyde thus obtained was dissolved in 130 mL of ethanol-water (4:1, v/v). Sodium borohydride (NaBH₄) (1.58 g, 40 mmol) was added in portions at 0° C. The resulting reaction mixture was stirred at rt for 2 h and then treated with 200 g of ice water. The mixture was extracted with ethyl acetate. The organic phase was washed with water and brine, dried (Na₂SO₄) and concentrated. The resulted residue was purified by flash chromatography on a silica gel column using 2:1, 1:1 and 1:2 hexanes-EtOAc as eluents to give 1.6 g of 5′-O-(tert-butyldiphenylsilyl)-3′-deoxy-3′-(hydroxymethyl)-2′-O-(2-methoxyethyl)-5-methyluridine as a white foam.

¹H NMR (CDCl₃): δ1.09 (s, 9H), 1.50 (s, 3H), 2.25 (bs, 1H), 2.52-2.78 (m, 1H), 3.38 (s, 3H), 3.52-4.25 (m, 10H), 5.86 (s, 1H), 7.38-7.70 (m, 11H), 9.95 (bs, 1H); ¹³C NMR (CDCl₃): δ12.1, 19.5, 27.1, 43.1, 58.2, 58.8, 63.1, 69.5, 71.6, 82.3, 86.1, 89.8, 110.5, 128.0, 130.2, 132.5, 133.2, 135.1, 135.3, 136.5, 150.5, 164.4; HRMS (FAB) m/z 569.268 (M+H)⁺(C₃₀H₄₁N₂O₇Si requires 569.268).

Step D: 5′-O-(tert-Butyldiphenylsilyl)-3′-deoxy-3′-(iodomethyl)-2′-O-(2-methoxyethyl)-5-methyluridine

To a solution of 5′-O-(tert-butyldiphenylsilyl)-3′-deoxy-3′-(hydroxymethyl)-2′-O-(2-methoxyethyl)-5-methyluridine (1.34 g, 2.35 mmol) in 25 mL of anhydrous DMF under stirring was added sequentially at 0° C. 2,6-lutidine (0.55 mL, 0.51 g, 4.7 mmol, 2.0 equiv) and methyl triphenoxy-phosphonium iodide (1.28 g, 2.83 mmol). The resulting reaction mixture was stirred at 0° C. for 1 h and at rt for 2 h. The reaction mixture was diluted with 10 mL of ethyl acetate and washed twice with 0.1 N Na₂S₂O₃ aqueous solution to remove iodine. The organic phase was further washed with aqueous NaHCO3 solution, water, and brine. The aqueous phases were back extracted with ethyl acetate. The combined organic phases were dried (Na₂SO₄) and concentrated. The resulting residue was purified by flash chromatography on a silica gel column using 5:1, 3:1 and then 1:1 hexanes-EtOAc to provide 1.24 g of 5′-O-(tert-butyldiphenylsilyl)-3′-deoxy-3′-(iodomethyl)-2′-O-(2-methoxyethyl)-5-methyluridine as a white foam.

¹H NMR (CDCl₃): δ1.13 (s, 9H), 1.62 (s, 3H), 2.64-2.85 (m, 2H), 3.20-3.35 (m, 1H), 3.38 (s, 3H), 3.50-4.25 (m, 8H), 5.91 (s, 1H), 7.32-7.50 (m, 6H), 7.60 (s, 1H), 7.62-7.78 (m, 4H), 10.46 (s, 1H); ¹³C NMR (CDCl₃): δ12.4, 19.5, 27.2, 45.0, 58.0, 62.5, 70.3, 71.9, 83.3, 85.6, 88.9, 110.5, 128.1, 128.2, 130.1, 130.3, 132.4, 132.9, 135.0, 135.4, 135.6, 150.7, 164.7; HRMS (FAB) m/z 679.172 (M+H)⁺ (C₃₀H₄₀IN₂O₆Si requires 679.170).

Step E: 3′-Deoxy-3′-(iodomethyl)-2′-O-(2-methoxyethyl)-5-methyluridine

A solution of 5′-O-(tert-butyldiphenylsilyl)-3′-deoxy-3′-(iodomethyl)-2′-O-(2-methoxyethyl)-5-methyluridine (1.12 g, 1.65 mmol) and triethylamine trihydrofluoride (1.1 mL, 1.1 g, 6.7 mmol) in 20 mL of THF was stirred at rt for 24 h. The reaction mixture was diluted with 50 mL of ethyl acetate and washed with water and brine. The organic phase was dried (Na₂SO₄) and concentrated. The residue was purified by flash chromatography on a silica gel column. Gradient elution with 2:1, 1:2 and then 1:3 hexanes-EtOAc provided 504 mg of the title compound as a white foam.

¹H NMR (CD₃OD): δ1.87 (s, 3H), 2.47-2.75 (m, 1H), 3.18-3.37 (m, 2H), 3.40 (s, 3H), 3.59-3.70 (m, 2H), 3.71-3.90 (m, 2H), 3.92-4.17 (m, 4H), 5.87 (s, 1H), 8.17 (s, 1H); ¹³C NMR (CD₃OD): δ12.5, 45.2, 59.2, 60.9, 71.0, 72.9, 85.4, 87.3, 89.7, 110.5, 138.0, 152.1, 166.6; HMS (FAB) m/z 441.053 (M+H)⁺ (C₁₄H₂₂IN₂O₆ requires 441.052). ps Step F: 3′-Deoxy-5′-O-(4-methoxytrityl)-3′-(iodomethyl)-2′-O-(2-methoxyethyl)-5-methyluridine

A mixture of 3′-deoxy-3′-(iodomethyl)-2′-O-(2-methoxyethyl)-5-methyluridine (472 mg, 1.1 mmol), diisopropylethylamine (0.79 mL, 0.586 g, 4.5 mmol), and p-anisyl chlorodiphenyl methane (4′-methoxytrityl chloride, MMT-Cl) (1.32 g, 4.27 mmol) in 6 mL of ethyl acetate and 4 mL of THF was stirred at rt for 48 h. The reaction mixture was diluted with ethyl acetate and washed with water, followed by brine. The organic phase was dried (Na₂SO₄) and concentrated. The crude product was purified by flash chromatography on a silica gel column. Gradient elution with 3:1, 2:1, 1:1, and then 1:3 hexanes-EtOAc provided 690 mg of the title compound as a white foam.

¹H NMR (CDCl₃): δ1.46 (s, 3H), 2.70-2.89 (m, 2H), 3.19-3.31 (m, 2H), 3.39 (s, 3H), 3.58-3.70 (m, 3H), 3.80 (s, 3H), 3.80-3.94 (m, 1H), 4.05-4.25 (m, 3H), 5.89 (s, 1H), 6.85 (s, 1H), 6.89 (s, 1H), 7.24-7.48 (m, 12H), 7.78 (s, 1H), 9.69 (s, 1H); ¹³C NMR (CDCl₃): δ12.3, 45.3, 55.3, 58.9, 61.6, 70.2, 71.9, 82.6, 85.6, 87.1, 89.1, 110.5, 113.4, 127.4, 128.2, 128.4, 130.5, 134.7, 135.3, 143.6, 143.7, 150.5, 158.9, 164.6. HRMS (FAB) m/z 735.155 (M+Na)⁺ (C₃₄H₃₇IN₂O₇Na requires 735.154).

Step G: 3′-Deoxy-5′-O-(4-methoxytrityl)-3′-methyl-2′-O-(2-methoxyethyl)-5-methyluridine

A mixture of ammonium phosphinate (410 mg, 5.1 mmol) and 1,1,1,3,3,3-hexamethyldisilazane (1.18 mL, 0.90 g, 5.59 mmol) was heated at 100-110° C. for 2 h under nitrogen atmosphere with condenser. The intermediate BTSP(bis[trimethylsilyl]phosphinate) was cooled to 0° C. and 5 mL of dichloromethane was injected. To this mixture was injected a solution of 3′-deoxy-5′-O-(4-methoxytrityl)-3′-(iodomethyl)-2′-O-(2-methoxyethyl)-5-methyluridine (0.78 g, 1.1 mmol) and diisopropylethylamine (0.39 mL, 287 mg, 2.23 mmol) in 7 mL of dichloromethane. After the reaction mixture was stirred at rt overnight, a mixture of THF-MeOH-NEt₃ (3/6/0.3 mL) was added and continued to stir for 1 h. The reaction mixture was filtered through a pad of celite and washed with dichloromethane. The solvent was evaporated and the residue was purified by flash chromatography on a silica gel column using 2:1, 1:1, and then 1:2 hexanes-EtOAc as eluent providing 380 mg of the title compound.

¹H NMR (CDCl₃): δ0.97 (d, 3H, J=6.8 Hz), 1.41 (s, 3H), 2.35-2.55 (m, 1H), 3.27 (dd, 1H, J=11.0, 3.0 Hz), 3.37 (s, 3H), 3.54-3.68 (m, 3H), 3.79 (s, 3H), 3.75-3.87 (m, 1H), 3.94 (d, 1H, J=5.0 Hz), 4.03-4.16 (m, 2H), 5.84 (s, 1H), 6.83 (s, 1H), 6.87 (s, 1H), 7.20-7.37 (m, 8H), 7.39-7.50 (m, 4H), 7.86 (s, 1H), 9.50 (s, 1H); ¹³C NMR (CDCl₃): δ8.7, 12.1, 35.6, 55.3, 59.0, 61.7, 69.8, 72.1, 85.4, 86.4, 86.7, 89.8, 110.0, 113.3, 127.2, 128.0, 128.4, 130.4, 135.0, 135.7, 143.9, 150.5, 158.8, 164.6.

HRMS (FAB) m/z 609.256 (M+Na)⁺ (C₃₄H₃₈N₂O₇Na requires 609.257).

Step H: 3′-Deoxy-3′-methyl-2′-O-(2-methoxyethyl)-5-methyluridine

Trifluoroacetic acid (1.5 mL) was added dropwise to a stirred solution of 3′-deoxy-5′-O-(4-methoxytrityl)-3′-methyl-2′-O-(2-methoxyethyl)-5-methyluridine (370 mg, 0.63 mmol) in 50 mL of chloroform at 0° C. The mixture was stirred at rt for 30 min, concentrated, and then dissolved in ethyl acetate. The solution was washed with dilute sodium bicarbonate and brine. The organic phase was dried (Na₂SO₄) and concentrated. The resulting residue was purified by flash chromatography on a silica gel column. Elution with 1:1, 1:3 and then 0:1 hexanes-EtOAc provided 170 mg of the title compound as a white foam.

¹H NMR (CDCl₃): δ1.03 (d, 3H, J=6.8 Hz), 1.83 (s, 3H), 2.20-2.40 (m, 1H), 3.10-3.28 (m, 1H), 3.35 (s, 3H), 3.50-4.15 (m, 10H), 5.81 (s, 1H), 7.89 (s, 1H), 9.77 (s, 1H); ¹³C NMR (CDCl₃): δ8.9, 12.4, 34.7, 59.0, 60.6, 69.7, 72.0, 86.3, 89.8, 109.7, 136.9, 150.4, 164.7. HRMS (FAB) m/z 315.154 (M+H)⁺ (C₄H₂₃N₂O₆ requires 315.155).

### Example 50

2′-Amino-2′-deoxyuridine

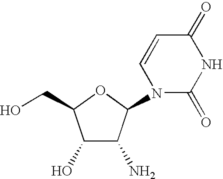

This compound was prepared following the procedures described in J. Org. Chem. 61: 781 (1996).

### Example 51

3′-Deoxyuridine

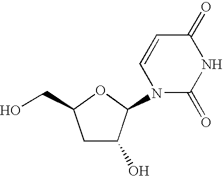

This compound was obtained from commercial sources.

### Example 52

2′-C-Methyladenosine

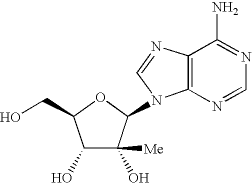

This compound was prepared following the conditions described in J. Med. Chem. 41: 1708 (1998).

### Example 53

3′-Deoxyadenosine (Cordycepin)

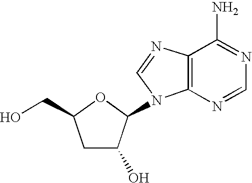

This compound was obtained from commercial sources.

### Example 54

3′-Amino-3′-deoxyadenosine

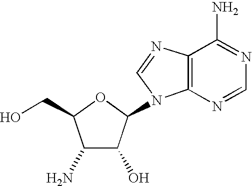

This compound was prepared following the conditions described in Tetrahedron Lett. 30: 2329 (1989).

### Example 55

8-Bromoadenosine

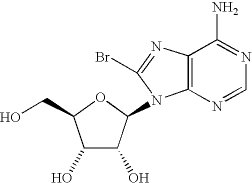

This compound was obtained from commercial sources.

### Example 56

2′-O-Methyladenosine

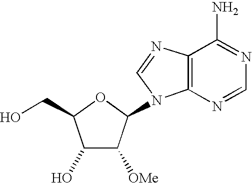

This compound was obtained from commercial sources.

### Example 57

3′-Deoxy-3′-fluoroadenosine

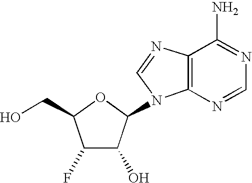

This compound was prepared following the procedures described in J. Med. Chem. 34: 2195 (1991).

### Example 58

6-Methyl-9-(□-D-ribofuranosyl)-9H-purine

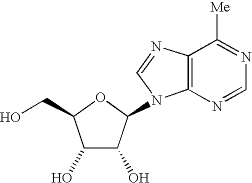

This compound was prepared following the procedures described in Nucleosides, Nucleotides, Nucleic Acids 19: 1123 (2000).

### Example 59

2′,3′,5′-tri-O-acetyl-8-methylsulfonyladenosine

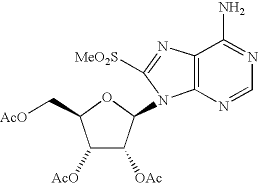

### Example 60

1-Methyl-9-[2,3,5-tri-O-(p-toluoyl)-β-D-ribofuranosyl]-9H-purine-6(1H)-thione

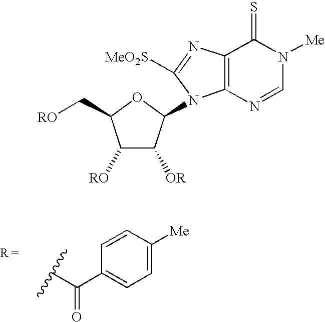

### Example 61

4-Amino-7-(2-C-methyl-β-D-arabinofuranosyl)-7H-pyrrolo[2,3-d]pyrimidine

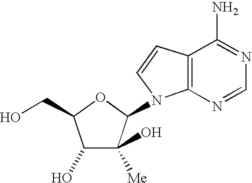

To CrO₃ (1.57 g, 1.57 mmol) in dichloromethane (DCM) (10 mL) at 0° C. was added acetic anhydride (145 mg, 1.41 mmol) and then pyridine (245 mg, 3.10 mmol). The mixture was stirred for 15 min, then a solution of 7-[3,5-O-[1,1,3,3-tetrakis(1-methylethyl)- 1,3-disiloxanediyl]-□-D-ribofuranosyl]-7H-pyrrolo[2,3-d]pyrimidin-4-amine [for preparation, see J. Am. Chem. Soc. 105: 4059 (1983)] (508 mg, 1.00 mmol) in DCM (3 mL) was added. The resulting solution was stirred for 2 h and then poured into ethyl acetate (10 mL), and subsequently filtered through silica gel using ethyl acetate as the eluent. The combined filtrates were evaporated in vacuo, taken up in diethyl ether/THF (1:1) (20 mL), cooled to −78° C. and methylmagnesium bromide (3M, in THF) (3.30 mL, 10 mmol) was added dropwise. The mixture was stirred at −78° C. for 10 min, then allowed to come to room temperature (rt) and quenched by addition of saturated aqueous ammonium chloride (10 mL) and extracted with DCM (20 mL). The organic phase was evaporated in vacuo and the crude product purified on silica gel using 5% methanol in dichloromethane as eluent. Fractions containing the product were pooled and evaporated in vacuo. The resulting oil was taken up in THF (5 mL) and tetrabutylammonium fluoride (TBAF) on silica (1.1 mmol/g on silica) (156 mg) was added. The mixture was stirred at rt for 30 min, filtered, and evaporated in vacuo. The crude product was purified on silica gel using 10% methanol in dichloromethane as eluent. Fractions containing the product were pooled and evaporated in vacuo to give the desired compound (49 mg) as a colorless solid.

¹H NMR (DMSO-d₆): □ 1.08 (s, 3H), 3.67 (m, 2H), 3.74 (m, 1H), 3.83 (m, 1H), 5.19 (m, 1H), 5.23 (m, 1H), 5.48 (m, 1H), 6.08 (1H, s), 6.50 (m, 1H), 6.93 (bs, 2H), 7.33 (m, 1H), 8.02 (s, 1H).

### Example 62

4-Amino-7-(2-C-methyl-β-D-ribofuranosyl)-7H-pyrrolo[2,3-d]pyrimidine

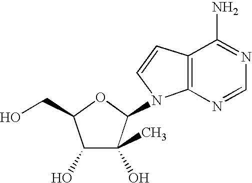

Step A: 3,5-Bis-O-(2,4-dichlorophenylmethyl)-1-O-methyl-□-D-ribofuranose

A mixture of 2-O-acetyl-3,5-bis-O-(2,4-dichlorophenylmethyl)-1-O-methyl-□-D-ribofuranose [for preparation, see: Helv. Chim. Acta 78: 486 (1995)] (52.4 g, 0.10 mol) in methanolic K₂CO₃ (500 mL, saturated at rt) was stirred at room temperature for 45 min. and then concentrated under reduced pressure. The oily residue was suspended in CH₂Cl₂ (500 mL), washed with water (300 mL+5×200 mL) and brine (200 mL), dried (Na₂SO₄), filtered, and concentrated to give the title compound (49.0 g) as colorless oil, which was used without further purification in Step B below.

¹H NMR (DMSO-d₆): δ3.28 (s, 3H, OCH₃), 3.53 (d, 2H, J_{5,4}=4.5 Hz, H-5a, H-5b), 3.72 (dd, 1H, J_{3,4}=3.6 Hz, J_{3,2}=6.6 Hz, H-3), 3.99 (ddd, 1H, J_{2,1}4.5 Hz, J_{2,OH-2}=9.6 Hz, H-2), 4.07 (m, 1H, H-4), 4.50 (s, 2H, CH₂Ph), 4.52, 4.60 (2d, 2H, J_{gem}=13.6 Hz, CH₂Ph), 4.54 (d, 1H, OH-2), 4.75 (d, 1H, H-1), 7.32-7.45, 7.52-7.57 (2m, 10H, 2Ph).

¹³C NMR (DMSO-d₆) δ55.40, 69.05, 69.74, 71.29, 72.02, 78.41, 81.45, 103.44, 127.83, 127.95, 129.05, 129.28, 131.27, 131.30, 133.22, 133.26, 133.55, 133.67, 135.45, 135.92.

Step B: 3,5-Bis-O-(2,4-dichlorophenylmethyl)-1-O-methyl-□-D-erythro-pentofuranos-2-ulose

To an ice-cold suspension of Dess-Martin periodinane (50.0 g, 118 mmol) in anhydrous CH₂Cl₂ (350 mL) under argon (Ar) was added a solution of the compound from Step A (36.2 g, 75 mmol) in anhydrous CH₂Cl₂ (200 mL) dropwise over 0.5 h. The reaction mixture was stirred at 0° C. for 0.5 h and then at room temperature for 3 days. The mixture was diluted with anhydrous Et₂O (600 mL) and poured into an ice-cold mixture of Na₂S₂O₃.5H₂O (180 g) in saturated aqueous NaHCO₃ (1400 mL). The layers were separated, and the organic layer was washed with saturated aqueous NaHCO₃ (600 mL), water (800 mL) and brine (600 mL), dried (MgSO₄), filtered and evaporated to give the title compound (34.2 g) as a colorless oil, which was used without further purification in Step C below.

¹H NMR (CDCl₃) δ3.50 (s, 3H, OCH₃), 3.79 (dd, 1H, J_{5a,5b}=11.3 Hz, J_{5a,4}=3.5 Hz, H-5a), 3.94 (dd, 1H, J_{5b,4}=2.3 Hz, H-5b), 4.20 (dd, 1H, J_{3,1}=1.3 Hz, J_{3,4}=8.4 Hz, H-3), 4.37 (ddd, 1H, H-4), 4.58, 4.69 (2d, 2H, J_{gem}=13.0 Hz, CH₂Ph), 4.87 (d, 1H, H-1), 4.78, 5.03 (2d, 2H, J_{gem}=12.5 Hz, CH₂Ph), 7.19-7.26, 7.31-7.42 (2m, 10H, 2Ph).

¹³C NMR (DMSO-d₆) δ55.72, 69.41, 69.81, 69.98, 77.49, 78.00, 98.54, 127.99, 128.06, 129.33, 129.38, 131.36, 131.72, 133.61, 133.63, 133.85, 133.97, 134.72, 135.32, 208.21.

Step C: 3,5-Bis-O-(2,4-dichlorophenylmethyl)-2-C-methyl-1-O-methyl-□-D-ribofuranose

To a solution of MeMgBr in anhydrous Et₂O (0.48 M, 300 mL) at −55° C. was added dropwise a solution of the compound from Step B (17.40 g, 36.2 mmol) in anhydrous Et₂O (125 mL). The reaction mixture was allowed to warm to −30° C. and stirred for 7 h at −30° C. to −15° C., then poured into ice-cold water (500 mL) and the mixture vigorously stirred at room temperature for 0.5 h. The mixture was filtered through a Celite pad (10×5 cm) which was thoroughly washed with Et₂O. The organic layer was dried (MgSO₄), filtered and concentrated. The residue was dissolved in hexanes (˜30 mL), applied onto a silica gel column (10×7 cm, prepacked in hexanes) and eluted with hexanes and hexanes/EtOAc (9/1) to give the title compound (16.7 g) as a colorless syrup.

¹H NMR (CDCl₃): δ1.36 (d, 3H, J_{Me, OH}=0.9 Hz, 2C-Me), 3.33 (q, 1H, OH), 3.41 (d, 1H, J_{3,4}=3.3 Hz), 3.46 (s, 3H, OCH₃), 3.66 (d, 2H, J_{5,4}=3.7 Hz, H-5a, H-5b), 4.18 (apparent q, 1H, H-4), 4.52 (s, 1H, H-1), 4.60 (s, 2H, CH₂Ph), 4.63, 4.81 (2d, 2H, J_{gem}=13.2 Hz, CH₂Ph), 7.19-7.26, 7.34-7.43 (2m, 10H, 2Ph).

¹³C NMR (CDCl₃): δ24.88, 55.45, 69.95, 70.24, 70.88, 77.06, 82.18, 83.01, 107.63, 127.32, 129.36, 130.01, 130.32, 133.68, 133.78, 134.13, 134.18, 134.45, 134.58.

Step D: 4-Chloro-7-[3,5-bis-O-(2,4-dichlorophenylmethyl)-2-C-methyl-β-D-ribofuranosyl]-7H-pyrrolo[2,3-d]pyrimidine

To a solution of the compound from Step C (9.42 g, 19 mmol) in anhydrous dichloromethane (285 mL) at 0° C. was added HBr (5.7 M in acetic acid, 20 mL, 114 mmol) dropwise. The resulting solution was stirred at 0° C. for 1 h and then at rt for 3 h, evaporated in vacuo and co-evaporated with anhydrous toluene (3×40 mL). The oily residue was dissolved in anhydrous acetonitrile (50 mL) and added to a solution of sodium salt of 4-chloro-1H-pyrrolo[2,3-d]pyrimidine [for preparation see: J. Chem. Soc.: 131 (1960)] in acetonitrile [generated in situ from 4-chloro-1H-pyrrolo[2,3-d]pyrimidine (8.76 g, 57 mmol) in anhydrous acetonitrile (1000 mL), and NaH (60% in mineral oil, 2.28 g, 57 mmol), after 4 h of vigorous stirring at rt]. The combined mixture was stirred at rt for 1 day, and then evaporated to dryness. The residue was suspended in water (250 mL) and extracted with EtOAc (2×500 mL). The combined extracts were washed with brine (300 mL), dried over Na₂SO₄, filtered and evaporated. The crude product was purified on a silica gel column (10 cm×10 cm) using ethyl acetate/hexane (1:3 and 1:2) as the eluent. Fractions containing the product were combined and evaporated in vacuo to give the desired product (5.05 g) as a colorless foam.

¹H NMR (CDCl₃): δ0.93 (s, 3H, CH₃), 3.09 (s, 1H, OH), 3.78 (dd, 1H, J_{5′,5″}=10.9 Hz, J_{5′,4}=2.5 Hz, H-5′), 3.99 (dd, 1H, J_{5″,4}=2.2 Hz, H-5″), 4.23-4.34 (m, 2H, H-3′, H-4′), 4.63, 4.70 (2d, 2H, J_{gem}=12.7 Hz, CH₂Ph), 4.71, 4.80 (2d, 2H, J_{gem}=12.1 Hz,CH₂Ph), 6.54 (d, 1H, , J_{5,6}=3.8 Hz, H-5), 7.23-7.44 (m, 10H, 2Ph).

¹³C NMR (CDCl₃): δ21.31, 69.10, 70.41, 70.77, 79.56, 80.41, 81.05, 91.11, 100.57, 118.21, 127.04, 127.46, 127.57, 129.73, 129.77, 130.57, 130.99, 133.51, 133.99, 134.33, 134.38, 134.74, 135.21, 151.07, 151.15 152.47.

Step E: 4-Chloro-7-(2-C-methyl-β-D-ribofuranosyl)-7H-pyrrolo[2,3-d]pyrimidine

To a solution of the compound from Step D (5.42 g, 8.8 mmol) in dichloromethane (175 mL) at −78° C. was added boron trichloride (1M in dichloromethane, 88 mL, 88 mmol) dropwise. The mixture was stirred at −78° C. for 2.5 h, then at −30° C. to −20° C. for 3 h. The reaction was quenched by addition of methanol/dichloromethane (1:1) (90 mL) and the resulting mixture stirred at −15° C. for 30 min., then neutralized with aqueous ammonia at 0° C. and stirred at rt for 15 min. The solid was filtered and washed with CH₂Cl₂/MeOH (1/1, 250 mL). The combined filtrate was evaporated, and the residue was purified by flash chromatography over silica gel using CH₂Cl₂ and CH₂Cl₂:MeOH (99:1, 98:2, 95:5 and 90:10) gradient as the eluent to furnish desired compound (1.73 g) as a colorless foam, which turned into an amorphous solid after treatment with MeCN.

¹H NMR (DMSO-d₆) δ0.64 (s, 3H, CH₃), 3.61-3.71 (m, 1H, H-5′), 3.79-3.88 (m, 1H, H-5″), 3.89-4.01 (m, 2H, H-3′, H-4′), 5.15-5.23 (m, 3H, 2′-OH, 3′-OH, 5′OH), 6.24 (s, 1H, H-1′), 6.72 (d, 1H, J_{5,6}=3.8 Hz, H-5), 8.13 (d, 1H, H-6), 8.65 (s, 1H, H-2).

¹³C NMR (DMSO-d₆) δ20.20, 59.95, 72.29, 79.37, 83.16, 91.53, 100.17, 117.63, 128.86, 151.13, 151.19, 151.45.

Step F: 4-Amino-7-(2-C-methyl-β-D-ribofuranosyl)-7H-pyrrolo[2,3-d]pyrimidine

To the compound from Step E (1.54 g, 5.1 mmol) was added methanolic ammonia (saturated at 0° C.; 150 mL). The mixture was heated in a stainless steel autoclave at 85° C. for 14 h, then cooled and evaporated in vacuo. The crude mixture was purified on a silica gel column with CH₂C₂/MeOH (9/1) as eluent to give the title compound as a colorless foam (0.8 g), which separated as an amorphous solid after treatment with MeCN. The amorphous solid was recrystallized from methanol/acetonitrile; m.p. 222° C.

¹H NMR (DMSO-d₆) δ0.62 (s, 3H, CH₃), 3.57-3.67 (m, 1H, H-5′), 3.75-3.97 (m, 3H, H-5″, H-4′, H-3′), 5.00 (s, 1H, 2′-OH), 5.04 (d, 1H, J_{3′OH,3′}=6.8 Hz, 1H, J_{5′OH,5′,5″}=5.1 Hz, 5′-OH), 6.11 (s, 1H, H-1′), 6.54 (d, 1H, J_{5,6}=3.6 Hz, H-5), 6.97 (br s, 2H, NH₂), 7.44 (d, 1H, H-6), 8.02 (s, 1H, H-2).

¹³C NMR (DMSO-d₆) δ20.26, 60.42, 72.72, 79.30, 82.75, 91.20, 100.13, 103.08, 121.96, 150.37, 152.33, 158.15.

LC-MS: Found: 279.10 (M-H⁺); calc. for C₁₂H₁₆N₄O₄+H⁺: 279.11.

### Example 63

4-Amino-7-(3-deoxy-3-methyl-□-D-ribofuranosyl)-7H-pyrrolo[2,3-d]pyrimidin-5-carboxamide

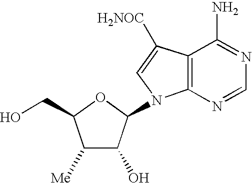

Step A: 4-Amino-6-bromo-7-(2-O-acetyl-5-O-benzoyl-3-deoxy-3-methyl-□-D-ribofuranosyl)-7H-pyrrolo[2,3-d]pyrimidin-5-carbonitrile

BSA (0.29 mL, 2.0 mmol) was added into a stirred suspension of 4-amino-6-bromo-5-cyano-1H-pyrrolo[2,3-d]pyrimidine (0.24 g, 1 mmol; prepared according to Nucleic Acid Chemistry, Part IV, Townsend, L. B. and Tipson, R. S.; Ed.; Wiley-Interscience: New York, 1991, pp. 16-17 and Synthetic Commun. 1998, 28, 3835) in dry acetonitrile (10 mL) at room temperature under argon. After 15 min, 1,2-di-O-acetyl-5-O-benzoyl-3-deoxy-3-methyl-D-ribofuranose (J. Med. Chem. (1976), 19, 1265) (0.36 g, 1.0 mmol) was added along with TMSOTf (0.54 g, 3 mmol). The mixture was stirred at room temperature for 5 min and then at 80° C. for 0.5 h. The solution was cooled, diluted with ethyl acetate (50 mL) and poured into ice-cold saturated aqueous NaHCO₃ (15 mL). The layers were separated. The organic layer was washed with brine (15 mL), dried (Na₂SO₄) and then evaporated. The residue was purified on silica gel column using a solvent system of hexanes/ EtOAc: 3/1. Appropriate fractions were collected and evaporated to provide the title compound as colorless foam (0.21 g).

Step B: 4-Amino-7-(2-O-acetyl-5-O-benzoyl-3-deoxy-3-methyl-□-D-ribofuranosyl)-7H-pyrrolo[2,3-d]pyrimidin-5-carbonitrile

To a suspension of the title compound from Step A (183 mg, 0.35 mmol) in EtOH (9 mL) were added ammonium formate (0.23 g, 3.6 mmol) and 10% palladium on activated carbon (20 mg) and the mixture was heated at reflux for 1.5 h. The hot reaction mixture was filtered through Celite and washed with hot EtOH. The solvent was removed and the residue treated with MeOH. The pale yellow solid was filtered thus yielding 105 mg of pure title compound. The filtrate was evaporated and purified on a silica gel column with a solvent system of CH₂Cl₂/MeOH: 50/1 to afford an additional 63 mg of title compound as a white solid.

Step C: 4-Amino-7-(3-deoxy-3-methyl-□-D-ribofuranosyl)-7H-pyrrolo[2,3-d]pyrimidin-5-carboxamide

A mixture of the compound from Step B (51 mg, 0.12 mmol), ethanolic ammonia (5 mL, saturated at 0° C.), aqueous ammonia (5 mL, 30%) and aqueous hydrogen peroxide (1 mL, 35%) was stirred room temperature for 8 h. The solution was evaporated and the residue purified on silica gel column with a solvent system of CH₂Cl₂/MeOH: 10/1 to give the title compound as a white solid (28 mg).

¹H-MNR (CD₃OD): □ 1.12 (d, 3H, J=6.8 Hz), 2.40 (m, 1H), 3.76(dd, 1H, J₁=12.8 Hz, J₂=4.0 Hz), 3.94-4.04 (m, 2H), 4.33 (d, 1H, J=5.4 Hz), 6.13 (s, 1H), 8.11 (s, 1H), 8.16 (s, 1H).

### Example 64

4-Amino-7-(3-deoxy-□-D-ribofuranosyl)-7H-pyrrolo[2,3-d]pyrimidine-5-carboxamide

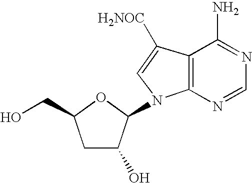

This compound was prepared following the procedures described in J. Med. Chem. 26: 25 (1983).

### Example 65

4-Amino-7-(□-D-ribofuranosyl)-7H-pyrrolo[2,3-d]pyrimidine-5-carboxamide (Sangivamycin)

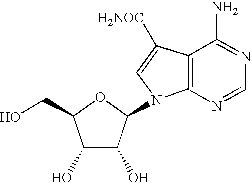

This compound was obtained from commercial sources.

### Example 66

7-(2-O-methyl-□-D-ribofuranosyl)-7H-pyrrolo[2,3-d]pyrimidine

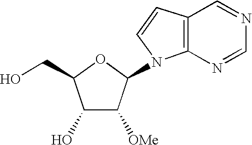

This compound was prepared following the procedures described in J. Org. Chem. 39: 1891 (1974).

### Example 67

4-Amino-7-(3-deoxy-3-fluoro-□-D-ribofuranosyl)-7H-pyrrolo[2,3-d]pyrimidine-5-carboxamide

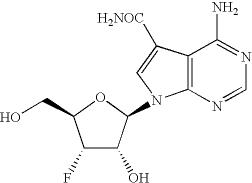

This compound was prepared following the procedures described in Chem. Pharm. Bull. 41: 775 (1993).

### Example 68

4-Amino-7-(3-deoxy-□-D-ribofuranosyl)-7H-pyrrolo[2,3-d]pyrimidine-5-carbonitrile

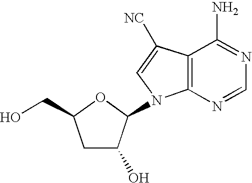

This compound was prepared following the procedures described in J. Med. Chem. 30: 481 (1987).

### Example 69

4-Amino-7-(2-O-methyl-□-D-ribofuranosyl)-7H-pyrrolo[2,3-d]pyrimidine

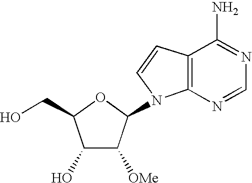

This compound was prepared following the procedures described in J. Org. Chem. 39: 1891 (1974).

### Example 70

3′-Amino-3′-deoxy-2′-O-methyladenosine

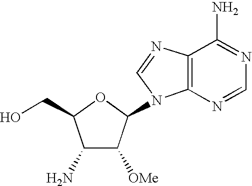

This compound is obtained by the methylation of appropriately protected 3′-amino-3′-deoxyadenosine derivative (Example 54).

### Example 71

4-Amino-7-(3-deoxy-□-D-ribofuranosyl)-7H-pyrrolo[2,3-d]pyrimidine

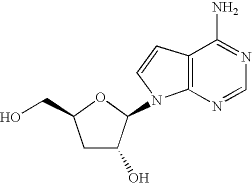

This compound was prepared following the following procedure described in Can. J. Chem. 55: 1251 (1977).

### Example 89

2-Amino-9-(□-D-arabinofuranosyl)-9H-purin-6(1H)-one

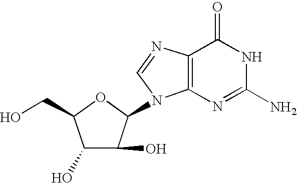

This compound was obtained from commercial sources.

### Example 90

3′-Deoxy-3′-methyliguanosine

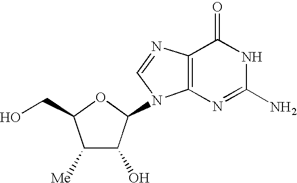

This compound was prepared following procedures described in U.S. Pat. No. 3,654,262 (1972).

### Example 91

2′-O-[4-(Imidazolyl-1)butyl]guanosine

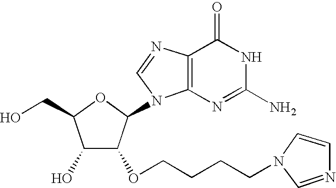

Step A: 2′-O-[4-(Imidazolyl-1)butyl]-2-amninoadenosine

A solution 2-aminoadenosine (7.36 g, 26 mmol) in dry DMF (260 mL) was treated portionwise with 60% NaH (3.92 g, 1000 mmol). After 1 hr., a solution of bromobutylimidazole (9.4 g, 286 mmol) in DMF (20ml) was added. After 16 hrs., the solution was conc. in vacuo, partitioned between H₂O/EtOAc and separated. The aqueous layer was evaporated, and the residue was chromatographed on silica gel (CHCl₃/MeOH) to afford the title nucleoside as a white solid; yield 4.2 g.

¹H NMR (DMSO-d₆): δ1.39 (t, 2H), 1.67 (t, 2H), 3.3-3.7 (m, 4H), 3.93 (m, 3H), 4.29 (m, 2H), 4.40 (d, 1H), 5.50 (5, 1H), 5.72 (d, 1H), 5.82 (bs, 2H), 6.72 (bs, 2H), (s, 1H), 7.08 (s, 1H), 7.57 (s, 1H). 7.91 (s, 1H).

Step B: 2′-O-[4-(Imidazolyl-1)butyl]guanosine

A mixture of the intermediate from Step A (3.2 g, 8 mmol) in H₂O (200 mL), DMSO (10 mL), trisodium phosphate (10 g), and adenosine deaminase (0.3 g) was stirred at room temperature and pH 7. The solution was filtered and and then evaporated. The resulting solid was crystallized from EtOAc/MeOH to afford the title compound as a white solid; yield 2.6 g.

¹H NMR (DMSO-d₆): δ1.39 (t, 2H), 1.67 (t, 2H), 3.3-3.7 (m, 4H), 3.93 (m, 3H), 4.29 (m, 2H), 5.10 (t, 1H), 5.20 (d, 1H), 5.79 (d, 1H), 6.50 (bs, 2H), 6.86 (s, 1H), 7.08 (s, 1H), 7.57 (s, 1H) 7.9 (s, 1H).

### Example 92

2′-Deoxy-2′-fluoroguanosine

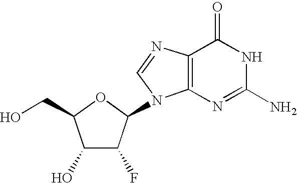

This compound was prepared following the conditions described in Chem. Pharm. Bull. 29: 1034 (1981).

### Example 93

2′-Deoxyguanosine

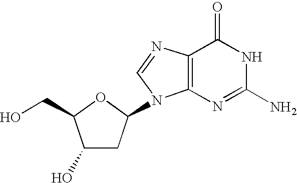

This compound was obtained from commercial sources.

### Example 94

2-Amino-7-(2-deoxy-2-fluoro-β-D-ribofuranosyl)-7H-pyrrolo[2,3-d]pyrimidin-4(3H)-one

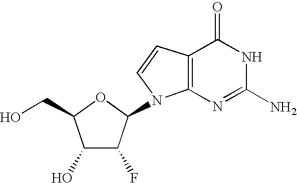

Step A: 2-Amino-4-chloro-7-(2,3,5-tri-O-benzyl-β-D-arabinofuranosyl)-7H-pyrrolo[2,3-d]-pyrimidine

To a suspension of 2-amino-4-chloro-1H-pyrrolo[2,3-d]pyrimidine [Liebigs Ann. Chem. 1: 137 (1983)] (3.03 g, 18 mmol) in anhydrous MeCN (240 mL), powdered KOH (85%; 4.2 g, 60 mmol) and tris[2-(2-methoxyethoxy)-ethyl]amine (0.66 mL, 2.1 mmol) were added and the mixture was stirred at room temperature for 10 min. Then a solution of 2,3,5-tri-O-benzyl-D-arabinofuranosyl bromide [prepared from corresponding 1-O-p-nitrobenzoate (11.43 g, 20.1 mmol) according to Seela et al., J. Org. Chem. (1982), 47, 226] in MeCN (10 mL) was added and stirring continued for another 40 min. Solid was filtered off, washed with MeCN (2×25 mL) and combined filtrate evaporated. The residue was purified on a silica gel column with a solvent system of hexanes/EtOAc: 7/1, 6/1 and 5/1. Two main zones were separated. From the more rapidly migrating zone was isolated the α anomer (0.74 g) and from the slower migrating zone the desired β anomer (4.01 g).

Step B: 2-Amino-7-(β-D-arabinofuranosyl)-4-chloro-7H-pyrrolo[2,3-d]pyrimidine

To a solution of the compound from Step A (4.0 g, 7 mmol) in CH₂Cl₂ (150 ml) at −78° C. was added a solution of 1.0 M BCl₃ in CH₂Cl₂ (70 mL, 70 mmol) during 45 min. The mixture was stirred at −78° C. for 3 h and at −20° C. for 2.5 h. MeOH-CH₂Cl₂ (70 mL, 1:1) was added to the mixture, which was then stirred at −20° C. for 0.5 h and neutralized with conc. aqueous NH₃ at 0° C. The mixture was stirred at room temperature for 10 min. and then filtered. The solid was washed with MeOH-CH₂Cl₂ (70 mL, 1:1) and the combined filtrate evaporated. The residue was purified on a silica gel column with a solvent system of CH₂Cl₂/MeOH: 20/1 to give the desired nucleoside (1.18 g) as a white solid.

Step C:2-Amino-7-[3,5-O-(1,1,3,3-tetraisopropyldisiloxane-1,3-diyl)-β-D-arabinofuranosyl]-4-chloro-7H-pyrrolo[2,3-d]pyrimidine

The compound from Step B (0.87 g, 2.9 mmol) and imidazole (0.43 g, 5.8 mmol were dissolved in DMF (3.5 mL). 1,3-Dichloro-1,1,3,3-tetraisopropyldisiloxane (1.0 mL) was added to the solution. The reaction mixture was stirred at room temperature for 1 h and then evaporated. The residue was partitioned between CH₂Cl₂ (150 mL) and water (30 mL). The layers were separated. The organic layer was dried (Na₂SO₄) and evaporated. The residue was purified on a silica gel column with a solvent system of hexanes/EtOAc: 7/1 and 5/1 to give the title compound (1.04 g).

Step D: 2-Amino-7-[2-O-acetyl-3,5-O-(1,1,3,3-tetraisopropyldisiloxane-1,3-diyl)-β-D-arabinofuranosyl]-4-chloro-7H-pyrrolo[2,3-d]pyrimidine

A mixture of the compound from Step C (0.98 g, 1.8 mmol) in MeCN (12 mL), Et₃N (0.31 mL) Ac₂O (0.21 mL) and DMAP (5 mg, 0.25 eq.) was stirred at room temperature for 5 h and then evaporated. The oily residue was dissolved in EtOAc (200 mL), washed with water (2×20 mL), dried (Na₂SO₄) and evaporated to yield pure title compound (1.12 g).

Step E:2-Amino-7-[2-O-acetyl-β-D-arabinofuranosyl]-4-chloro-7H-pyrrolo[2,3-d]pyrimidine

To an ice-cold solution of the compound from Step D (0.95 g, 1.63 mmol) in THF (10 mL) and AcOH (0.19 mL) was added dropwise 1.0 M tetrabutylammonium fluoride solution in THF (3.4 mL) and stirred at 0° C. for 15 min. The solution was concentrated and the oily residue applied onto a silica gel column packed in CH₂Cl₂ and eluted with CH₂Cl₂/MeOH: 50/1, 25/1 and 20/1. Appropriate fractions were pooled and evaporated to give the title nucleoside (0.56 g) as a white solid.

Step F: 2-Amino-7-[2-O-acetyl-3,5-di-O-(tetrahydro-2-pyranyl)-β-D-arabinofuranosyl]-4-chloro-7H-pyrrolo[2,3-d]pyrimidine

To a solution of the compound from Step E (0.5 g, 1.46 mmol) in CH₂Cl₂ (10 mL) and 3,4-dihydro-2-H-pyrane (0.67 mL) was added dropwise TMSI (30 μL, 0.2 mmol). The reaction mixture was stirred at room temperature for 1 h and then evaporated. The oily residue was purified on a silica gel column packed in a solvent system of hexanes/EtOAc/Et₃N: 75/25/1 and eluted with a solvent system of hexanes/EtOAc: 3/1. The fractions containing the product were collected and evaporated to give the desired compound (0.60 g).

Step G: 2-Amino-7-[3,5-di-O-(tetrahydro-2-pyranyl)-β-D-arabinofuranosyl]-4-chloro-7H-pyrrolo[2,3-d]pyrimidine

A mixture of the compound from Step F (0.27 g, 0.53 mmol) and methanolic ammonia (saturated at 0° C.; 10 mL) was kept overnight at 0° C. Evaporation of the solvent yielded the desired compound (0.25 g).

Step H: 2-Amino-7-[2-deoxy-2-fluoro-3,5-di-O-(tetrahydro-2-pyranyl)-(β-D-ribofuranosyl]-4-chloro-7H-pyrrolo[2,3-d]pyrimidine

To a solution of the compound from Step G (0.24 g, 0.51 mmol) in CH₂Cl₂ (5 mL) and pyridine (0.8 mL) at −60° C. was added diethylaminosulfur trifluoride (DAST; 0.27 mL) dropwise under Ar. The solution was stirred at −60° C. for 0.5 h, at 0° C. overnight and at room temperature for 3 h. The mixture was diluted with CH₂Cl₂ (25 mL) and poured into saturated aqueous NaHCO₃ (15 mL). The organic layer was washed with water (10 mL), dried (Na₂SO₄) and evaporated. The residue was purified on a silica gel column with a solvent system of hexanes/EtOAc: 5/1 to give the title compound (45 mg) as a pale yellow foam.

Step I: 2-Amino-7-(2-deoxy-2-fluoro-β-D-ribofuranosyl)-4-chloro-7H-pyrrolo[2,3-d]-pyrimidine

A solution of the compound from Step H (40 mg. 0.08 mmol) in EtOH (2 mL) was stirred with pyridinium p-toluenesulfonate (40 mg, 0.16 mmol) at 60° C. for 3 h. The mixture was then evaporated and the residue purified on a silica gel column with a solvent system of hexanes/EtOAc: 1/1 and 1/2 to give the desired compound (24 mg).

Step J: 2-Amino-7-(2-deoxy-2-fluoro-β-D-ribofuranosyl)-7H-pyrrolo[2,3-d]pyrimidin-4(3H)-one

A mixture of the compound from Step I (4 mg, 0.08 mmol) in 2N aqueous NaOH (1.2 mL) was stirred at reflux temperature for 1.5 h. The solution was cooled in an ice-bath, neutralized with 2 N aqueous HCl and evaporated to dryness. The residue was suspended in MeOH, mixed with silica gel and evaporated. The solid residue was placed onto a silica gel column (packed in a solvent system of CH₂Cl₂/MeOH: 10/1) which was eluted with a solvent system of CH₂Cl₂/MeOH: 10/1. The fractions containing the product were collected and evaporated to dryness to yield the title compound (20 mg) as a white solid.

¹H NMR (CD₃OD): δ3.73, 3.88 (2dd, 2H, J=12.4, 3.8, 2.6 Hz), 4.01 (m, 1H), 4.47 (ddd, 1H J=16.5, 6.6 Hz), 5.14 (ddd, 1H, J=5.3 , 4.7 Hz), 6.19 (dd, 1H, J=17.8, 3.0 Hz), 6.39 (d, 1H, J=3.6 Hz), 6.95 (d, 1H).

¹⁹F NMR (CD₃OD): δ−206.53 (dt).

### Example 95

2-Amino-7-(β-D-ribofuranosyl)-7H-pyrrolo[2,3-d]pyrimidin-4(3H)-one

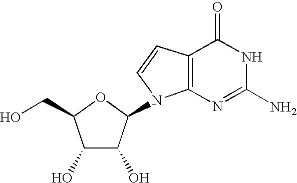

This compound was prepared following the procedures described in J. Chem. Soc. Perkin Trans. 1, 2375 (1989).

### Example 96

2-Amino-7-(2-deoxy-β-D-ribofuranosyl)-7H-pyrrolo[2,3-d]pyrimidin-4(3H)-one

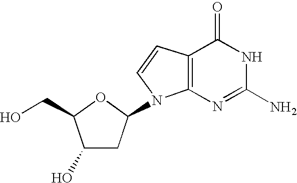

This compound was prepared following the procedures in Tetrahedron Lett. 28: 5107 (1987).

### Example 97

6-Amino-1-(2-O-methyl-β-D-ribofuranosyl)-1H-imidazo[4,5-c]pyridin-4(5H)-one

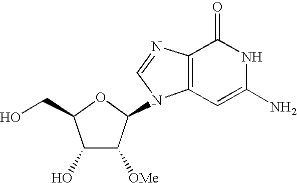

This compound was prepared in a manner similar to the preparation of 2-amino-7-(3-deoxy-3-fluoro-□-D-ribofuranosyl)-7H-pyrrolo[2,3-d]pyrimidin4(3H)-one (Example 23).

### Example 98

6-Amino-1-(2-deoxy-β-D-ribofuranosyl)-1H-imidazo[4,5-c]pyridin-4(5H)-one

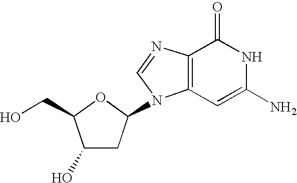

This compound was prepared following-the procedures described in J. Med. Chem. 26: 286 (1983).

### Example 99

6-Amino-1-(3-deoxy-3-methyl-β-D-ribofuranosyl)-1H-imidazo[4,5-c]pyridin-4(5H)-one

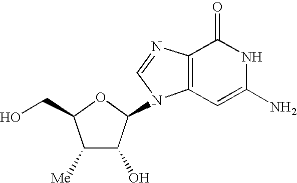

This compound was prepared in a manner similar to the preparation of 2-amino-7-(3-deoxy-3-fluoro-□-D-ribofuranosyl)-7H-pyrrolo[2,3-d]pyrimidin4(3H)-one (Example 23).

### Example 100

6-Amino-1-(2-deoxy-2-fluoro-β-D-ribofuranosyl)-1H-imidazo[4,5-c]pyridin-4(5H)-one

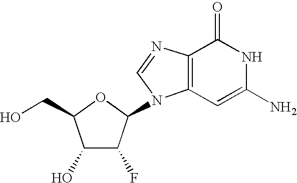

This compound was prepared in a manner similar to the preparation of 2-amino-7-(3-deoxy-3-fluoro-□-D-ribofuranosyl)-7H-pyrrolo[2,3-d]pyrimidin-4(3H-one (Example 23).

### Example 101

6-Amino-1-(β-D-arabinofuranosyl)-1H-imidazo[4,5-c]pyridin-4(5H)-one

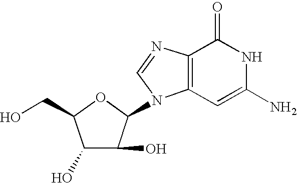

A preparation of this compound is given in Eur. Pat. Appln. 43722 A1 (1982).

### Example 102

2′-O-[2-(N,N-diethylaminooxy)ethyl]-5-methyluridine

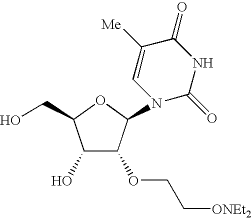

Step A: 5′-O-tert-Butyldiphenylsilyl-2′-O-(2-hydroxyethyl)-5-methyluridine

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-Butyldiphenylsilyl-O²-2′-anhydro-5-methyluridine (149 g, 0.311 mol) and sodium bicarbonate (0.074 g) were added with manual stirring. The reactor was sealed and heated in an oil bath until an internal temperature of 160° C. was reached and then maintained for 16 h (pressure <100 psig). The reaction vessel was cooled to ambient and opened. The reaction mixture was concentrated under reduced pressure (10 to 1 mm Hg) in a warm water bath (40-100° C.) with the more extreme conditions used to remove the ethylene glycol. The residue was purified by column chromatography (2 kg silica gel, ethyl acetate:hexanes gradient 1:1 to 4:1). The appropriate fractions were combined, stripped and dried to product as white crisp foam (84 g), contaminated starting material (17.4 g) and pure reusable starting material (20 g). TLC and NMR were consistent with 99% pure product.

¹H NMR (DMSO-d₆): δ1.05 (s, 9H), 1.45 (s, 3H), 3.54 4.10(m, 8H), 4.25 (m, 1 H), 4.80 (t, 1H), 5.18 (d, 2H), 5.95 (d, 1H), 7.35-7.75 (m, 11H), 11.42 (s, 1H).

Step B: 2′-O-[2-(2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol). It was then dried over P₂O₅ under high vacuum for two days at 40° C. The reaction mixture was flushed with argon and dry THF (369.8 mL) was added to get a clear solution. Diethyl azodicarboxylate (6.98 mL, 44.36 mmol) was added dropwise to the reaction mixture. The rate of addition was maintained such that resulting deep red coloration is just discharged before adding the next drop. After the addition was complete, the reaction was stirred for 4 h. By that time TLC showed the completion of the reaction (ethyl acetate/hexane, 60:40). The solvent was evaporated under vacuum. Residue obtained was placed on a flash silica gel column and eluted with ethyl acetate-hexane (60:40) to give the title compound as a white foam (21.8 g).

¹H NMR (DMSO-d₆): δ11.32 (s, 1H), 7.82 (m, 4H), 7.6-7.65 (m, 5H), 7.34-7.46 (m, 6H), 5.90 (d, 1H, J=6Hz), 5.18 (d, J=5.6 Hz), 4.31 (bs, 2H), 4.25 (m, 1H), 4.09 (t, 1H, J=5.6 Hz), 3.81-3.94 (m, 5H), 1.44 (s, 3H), 1.1 (s, 9H); ¹³C NMR (CDCl₃): δ11.8, 19.40, 26.99, 62.62, 68.36, 68.56, 77.64, 83.04, 84.14, 87.50, 110.93, 123.59, 127.86, 129.89, 132.45, 134.59, 134.89, 135.17, 150.50, 163.63, 163.97; MS [FAB] m/z 684 [M−H]⁻.

Step C: 5′-O-tert-Butyldiphenylsilyl-2′-O-[2-(acetaldoximinooxy)ethyl]-5-methyluridine

2′-O-[2-(2-Phthalimidoxy)ethyl]-5′-t-butyldiphenylsilyl-5-methyluridine (10 g, 14.6 mmol) was dissolved in CH₂Cl₂ (146 mL) and cooled to −10° C. in an isopropanol-dry ice bath. To this methylhydrazine (1.03 mL, 14.6 mmol) was added dropwise. Reaction mixture was stirred at −10° C. to 0 C. for 1 h. A white precipitate formed and was filtered and washed thoroughly with CH₂Cl₂ (ice cold). The filtrate was evaporated to dryness. Residue was dissolved in methanol (210 mL) and acetaldehyde (0.89 mL, 16 mmol) was added and stirred at room temperature for 12 h. Solvent was removed in vacuo and residue was purified by silica gel column chromatography using and ethyl acetate/hexane (6:4) as solvent system to yield the title compound (4.64 g).

¹H NMR (DMSO-d₆): δ1.02 (s, 9H), 1.44 (s, 3H), 1.69 (dd, 3H, J=5.6 Hz), 3.66 (m, 1H), 3.76 (m, 2H), 3.94 (m, 2H), 4.05 (s, 2H), 4.15 (m, 1H), 4.22 (m, 1H), 5.18 (d, 1H, J=6.0 Hz), 5.9 (dd, 1H, J=4.4 Hz), 7.36 (m, 1H), 7.40 (m, 7H), 7.63 (m, 5H), 11.38 (s, 1H), ¹³C NMR (CDCl₃): δ11.84, 15.05, 19.38, 26.97, 63.02, 68.62, 70.26, 71.98, 72.14, 82.72, 84.34, 87.02, 111.07, 127.89, 130.02, 134.98, 135.13, 135.42, 147.85, 150.51, 164.12; HRMS (FAB) Calcd for C₃₀H₃₉N₃O₇ SiNa^{⊕} 604.2455, found 604.2471.

Step D: 5′-O-tert-Butyldiphenylsilyl-2′-O-[2-(N,N-diethylaminooxy)ethyl]-5-methyluridine

5′-O-tert-Butyldiphenylsilyl-2′-O-[2-(acetaldoximinooxy)ethyl]-5-methyluridine (4.5 g, 7.74 mmol) was dissolved in 1M pyridinium p-toluenesulfonate (PPTS) in MeOH (77.4 mL). It was then cooled to 10° C. in an ice bath. To this mixture NaBH₃CN (0.97 g, 15.5 mmol) was added and the mixture was stirred at 10° C. for 10 minutes. Reaction mixture was allowed to come to room temperature and stirred for 4 h. Solvent was removed in vacuo to give an oil. Diluted the oil with ethyl acetate (100 mL), washed with water (75 mL), 5% NaHCO₃ (75 mL) and brine (75 mL). The organic phase was dried over anhydrous Na₂SO₄ and evaporated. Residue obtained was dissolved in 1M PPTS in MeOH (77.4 mL), acetaldehyde (0.48 mL, 8.52 mmol) was added and stirred at ambient temperature for 10 minutes. Then reaction mixture was cooled to 10° C. in an ice bath and NaBH₃CN (0.97 g, 15.50 mmol) was added and stirred at 10° C. for 10 minutes. Reaction mixture was allowed to come to room temperature and stirred for 4 h. Solvent was removed in vacuo to get an oil. The oil was dissolved in ethyl acetate (100 mL), washed with water (75 mL), 5% NaHCO₃ (75 mL) and brine (75 mL). The organic phase was dried over anhydrous Na₂SO₄ and evaporated to dryness. The residue obtained was purified by silica gel column chromatography and eluted with CH₂Cl₂/MeOH/NEt₃, 94:5:1 to give title compound (3.55 g) as a white foam.

¹H NMR (DMSO-d₆): δ0.95 (t, 6H, J=7.2 Hz), 1.03 (s, 9H), 1.43 (s, 3H), 2.58 (q, 4H, J=7.2 Hz), 3.59 (m, 1H), 3.73 (m, 3H), 3.81 (m, 1H), 3.88 (m, 1H), 3.96 (m, 2H), 4.23 (m, 1H), 5.21 (d, 1H, J=5.6 Hz), 5.95 (d, 1H, J=6.4 Hz), 7.43 (m, 7H), 7.76 (m, 4H), 11.39 (s, 1H); ¹³C NMR (CDCl₃): δ11.84, 19.35, 26.97, 52.27, 63.27, 68.81, 70.27, 72.27, 82.64, 84.47, 86.77, 111.04, 127.87, 130.01, 135.11, 135.41, 141.32, 150.48, 164.04; HRMS (FAB), Calcd for C₃₂H₄₅N₃O₇SiCs^{⊕}, 744.2081, found 744.2067.

Step E: 2′-O-[2-(N,N-diethylaminooxy)ethyl]-5-methyluridine

A mixture of triethlyamine trihydrogenfluoride (4.39 mL, 26.81 mmol) and triethylamine (1.87 mL, 13.41 mmol) in THF (53.6 mL) was added to 5′-O-tert-butyldiphenylsilyl-2′-O-[2-(N,N-diethylaminooxy)ethyl]-5-methyluridine (3.28 g, 5.36 mmol). The reaction mixture was stirred at room temperature for 18 h. Solvent was removed in vacuo. The residue was placed on a silica gel column and eluted with CH₂Cl₂/MeOH/NEt₃, 89:10:1, to yield the title compound (1.49 g).

¹H NMR (DMSO-d₆): δ0.97 (t, 6H, J=7.2 Hz), 1.75 (s, 3H), 2.58 (q, 4H, J=7.2 Hz), 3.55 (m, 4H), 3.66 (m, 2H), 3.83 (bs, 1H), 3.95 (t, 1H, J=5.6 Hz), 4.11 (q, 1H, J=4.8 Hz and 5.6 Hz), 5.05 (d, 1H, J=5.6 Hz), 5.87 (d, 1H, J=6.0 Hz), 7.75 (s, 1H), 11.31 (s, 1H); ¹³C NMR (CDCl₃): δ11.75, 12.27, 52.24, 61.31, 68.86, 70.19, 72.25, 81.49, 85.10, 90.29, 110.60, 137.79, 150.57, 164.37; HRMS (FAB) Calcd for C₁₆H₂₈N₃O₇^{⊕} 374.1927, found 374.1919.

### Example 103

1-(2-C-Methyl-β-D-arabinofuranosyl)uracil

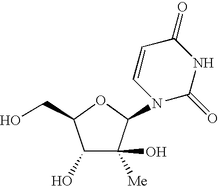

This compound was prepared following the procedures described in Chem. Pharm. Bull. 35: 2605 (1987).

### Example 104

5-Methyl-3′-deoxycytidine

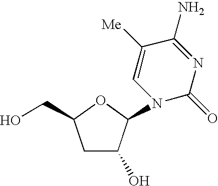

This compound was prepared following the procedures described in Chem. Pharm. Bull. 30: 2223 (1982).

### Example 105

2-Amino-2′-O-methyladenosine

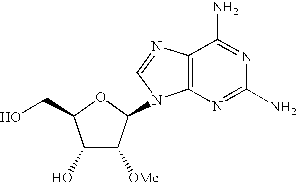

This compound was obtained from commercial sources.

### Example 106

2′-Deoxy-2′-fluoroadenosine

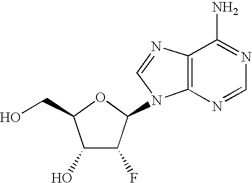

This compound was obtained from commercial sources.

### Example 107

3′-Deoxy-3′-fluoroadenosine

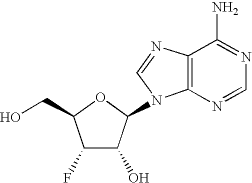

This compound was prepared following the procedures described in Nucleosides Nucleotides 10: 719 (1991).

### Example 108

3′-Deoxy-3′-methyladenosine

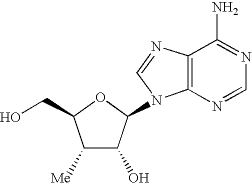

This compound was prepared following the procedures described in J. Med. Chem. 19:1265 (1976).

### Example 109

2-Amino-7-(2-deoxy-□-D-ribofuranosyl)-7H-pyrrolo[2,3-d]pyrimidine

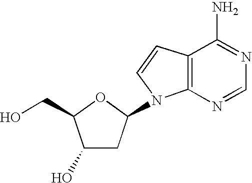

This compound was prepared following the procedures described in J. Am. Chem. Soc. 106: 6379 (1984).

### Example 110

4-Amino-7-(β-D-arabinofuranosyl)-7H-pyrrolo[2,3-d]pyrimidine

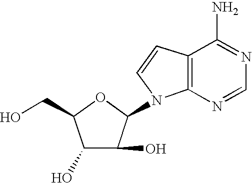

This compound is described in U.S. Pat. 4,439,604, which is incorporated by reference herein in its entirety.

### Example 111

4-Amino-1-(3-deoxy-3-fluoro-β-D-ribofuranosyl)-1H-imidazo[4,5-c]pyridine

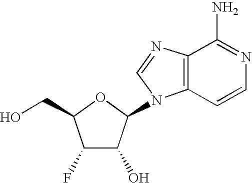

This compound can be prepared readily by the similar method described for the preparation of Example 24 except the nucleobase is 3-deazaadenine.

### Example 112

4-Amino-7-(β-D-ribofuranosyl)-7H-pyrrolo[2,3-d]pyrimidine (tubercidin)

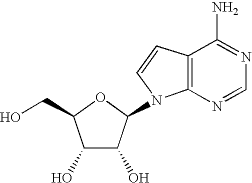

This compound was obtained from commercial sources.

### Example 113

4-Amino-1-(3-deoxy-β-D-ribofuranosyl)-1H-imidazo[4,5-c]pyridine

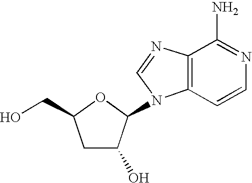

This compound is described in Acta Crystallogr., Sect. C: Cryst. Struct. Commun. C43: 1790 (1987).

### Example 114

4-Amino-1-(3-deoxy-3-methyl-β-D-ribofuranosyl)-1H-imidazo[4,5-c]pyridine

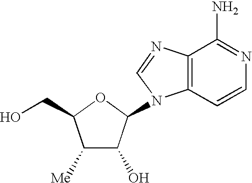

The procedure described earlier for Example 23 is used to synthesize this example by reacting the appropriately substituted 3-C-methyl-sugar intermediate with a protected 3-deazaadenine derivative.

### Example 115

4-Amino-1-β-D-ribofuranosyl-1H-imidazo[4,5-c]pyridine

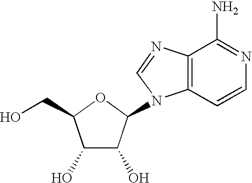

This compound was obtained from commercial sources.

### Example 116

9-(2-C-Methyl-β-D-arabinofuranosyl)adenine

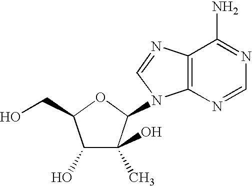

This compound is prepared from 4-amino-9-(3,5-bis-O-tert-butyldimethylsilyl-β-D-erythro-pentofuran-2-ulosyl)purine (J. Med. Chem. 1992, 35, 2283) by reaction with MeMgBr and deprotection as described in Example 61.

### Example 117

4-Amino-7-(2-C-ethyl-β-D-ribofuranosyl)-7H-pyrrolo[2,3-d]pyrimidine

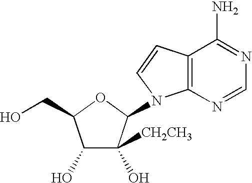

Step A: 3,5-Bis-O-(2,4-dichlorophenylmethyl)-2-C-ethyl-1-O-methyl-□-D-ribofuranose

To Et₂O (300 mL) at −78° C. was slowly added EtMgBr (3.0 M, 16.6 mL) and then dropwise the compound from Step B of Example 62 (4.80 g, 10.0 mmol) in anhydrous Et₂O (100 mL). The reaction mixture was stirred at −78° C. for 15 min, allowed to warm to −15° C. and stirred for another 2 h, and then poured into a stirred mixture of water (300 mL) and Et₂O (600 mL). The organic phase was separated, dried (MgSO₄), and evaporated in vacuo. The crude product was purified on silica gel using ethyl acetate/hexane (1:2) as eluent. Fractions containing the product were pooled and evaporated in vacuo to give the desired product (3.87 g) as a colorless oil.

Step B: 4-Chloro-7-[3,5-bis-O-(2,4-dichlorophenylmethyl)-2-C-ethyl-β-D-ribofuranosyl]-7H-pyrrolo[2,3-d]pyrimidine

To a solution of the compound from Step A (1.02 mg, 2.0 mmol) in dichloromethane (40 mL) was added HBr (5.7 M in acetic acid) (1.75 mL, 10.0 mmol) dropwise at 0° C. The resulting solution was stirred at rt for 2 h, evaporated in vacuo and co-evaporated twice from toluene (10 mL). The oily residue was dissolved in acetonitrile (10 mL) and added to a vigorously stirred mixture of 4-chloro-1H-pyrrolo[2,3-d]pyrimidine (307 mg, 2.00 mmol), potassium hydroxide (337 mg, 6.0 mmol) and tris[2-(2-methoxyethoxy)ethyl]amine (130 mg, 0.4 mmol) in acetonitrile (10 mL). The resulting mixture was stirred at room temperature overnight, and then poured into a stirred mixture of saturated ammonium chloride (100 mL) and ethyl acetate (100 mL). The organic layer was separated, washed with brine (100 mL), dried over MgSO₄, filtered and evaporated in vacuo. The crude product was purified on silica gel using ethyl acetate/hexane (1:2) as eluent to give the desired product (307 mg) as a colorless foam.

Step C: 4-Chloro-7-(2-C-ethyl-β-D-ribofuranosyl)-7H-pyrrolo[2,3-d]pyrimidine

To a solution of the compound from Step B (307 mg, 0.45 mmol) in dichloromethane (8 mL) was added boron trichloride (1M in dichloromethane) (4.50 mL, 4.50 mmol) at −78° C. The mixture was stirred at −78° C. for 1 h, then at −10° C. for 3 h. The reaction was quenched by addition of methanol/dichloromethane (1:1) (10 mL), stirred at −15° C. for 30 min, and neutralized by addition of aqueous ammonium hydroxide. The mixture was evaporated in vacuo and the resulting oil purified on silica gel using methanol/dichloromethane (1:9) as eluent. Fractions containing the product were pooled and evaporated in vacuo to give the desired product (112 mg) as a colorless foam.

Step D: 4-Amino-7-(2-C-ethyl-(β-D-ribofuranosyl)-7H-pyrrolo[2,3-d]pyrimidine

To the compound from Step C (50 mg, 0.16 mmol) was added saturated ammonia in methanol (4 mL). The mixture was stirred at 75° C. for 72 h in a closed container, cooled and evaporated in vacuo. The crude mixture was purified on silica gel using methanol/dichloromethane (1:9) as eluent. Fractions containing the product were pooled and evaporated in vacuo to give the desired product (29 mg) as a colorless powder.

¹H NMR (200 MHz, DMSO-d₆): □ 0.52 (t, 3H), 1.02 (m, 2H), 4.01-3.24 (m, 6H), 5.06 (m, 1H), 6.01 (s, 1H), 6.51 (d, 1H), 6.95 (s br, 2H), 6.70 (d, 1H), 7.99 (s, 1H).

LC-MS: Found: 295.2 (M+H⁺); calc. for C₁₃H₁₈N₄O₄+H⁺: 295.14.

### Example 118

2-Amino-7-(2-C-methyl-z,900 -D-ribofuranosyl)-7H-pyrrolo[2,3-d]pyrimdin-4(3H)-one

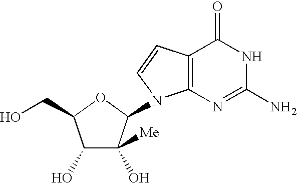

Step A: 2-Amino4-chloro-7-[3,5-bis-O-(2,4-dichlorophenylmethyl)-2-C-methyl- [symbol] -D-ribofuranosyl)-7H-pyrrolo[2,3-d]pyrimidine

To an ice-cold solution of product from Step C of Example 62 (1.27 g, 2.57 mmol) in CH₂Cl₂ (30 mL) was added HBr (5.7 M in acetic acid; 3 mL) dropwise. The reaction mixture was stirred at room temperature for 2 h, concentrated in vacuo and co-evaporated with toluene (2×15 mL). The resulting oil was dissolved in MeCN (15 mL) and added dropwise into a well-stirred mixture of 2-amino4-chloro-7H-pyrrolo[2,3-d]pyrimidine [for preparation see Heterocycles 35: 825 (1993)] (433 mg, 2.57 mmol), KOH (85%, powdered) (0.51 g, 7.7 mmol), tris-[2-(2-methoxyethoxy)ethyl]amine (165 μL, 0.51 mmol) in acetonitrile (30 mL). The resulting mixture was stirred at rt for 1h, filtered and evaporated. The residue was purified on a silica gel column using hexanes/EtOAc, 5/1, 3/1 and 2/1 as eluent to give the title compound as a colorless foam (0.65 g).

Step B: 2-Amino-4-chloro-7-(2-C-methyl- [symbol] -D-ribofuranosyl)-7H-pyrrolo[2,3-d]pyrimidine

To a solution of the product from Step A (630 mg, 1.0 mmol) in CH₂Cl₂ (20 mL) at −78° C. was added boron trichloride (1M in CH₂Cl₂) (10 mL, 10 mmol). The mixture was stirred at −78° C. for 2 h, then at −20° C. for 2.5 h. The reaction was quenched with CH₂Cl₂/MeOH (1:1) (10 mL), stirred at −20° C. for 0.5 h, and neutralized at 0° C. with aqueous ammonia. The solid was filtered, washed with CH₂Cl₂/MeOH (1:1) and the combined filtrate evaporated in vacuo. The residue was purified on a silica gel column with CH₂Cl₂/MeOH, 50/1 and 20/1 as eluent to give the title compound as a colorless foam (250 mg).

Step C: 2-Amino-7-(2-C-methyl- [symbol] -D-ribofuranosyl)-7H-pyrrolo[2,3-d]pyrimidin-4(3H)-one

A mixture of product from Step B (90 mg, 0.3 mmol) in aqueous NaOH (2N, 9 mL) was heated at reflux temperature for 5 h, then neutralized at 0° C. with 2 N aqueous HCl and evaporated to dryness. Purification on a silica gel column with CH₂Cl₂/MeOH, 5/1 as eluent afforded the title compound as a white solid (70 mg).

¹H NMR (200 MHz, CD₃OD): δ0.86 (s, 3H), 3.79 (m 1H), 3.90-4.05 (m, 3H), 6.06 (s, 1H), 6.42 (d, J=3.7 Hz, 1H), 7.05 (d, J=3.7 Hz, 1H).

### Example 119

2-Amino-4-cyclopropylamino-7-(2-C-methyl- [symbol] -D-ribofuranosyl)-7H-pyrrolo[2,3-d]pyrimidine

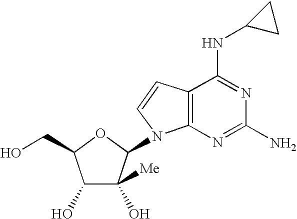

A solution of 2-amino-4-chloro-7-(2-C-methyl- [symbol] -D-ribofuranosyl)-7H-pyrrolo[2,3-d]pyrimidine (Example 118, Step B) (21 mg, 0.07 mmol) in cyclopropylamine (0.5 mL) was heated at 70° C. for two days, then evaporated to an oily residue and purified on a silica gel column with CH₂Cl₂/MeOH, 20/1, as eluent to give the title compound as a white solid (17 mg).

¹H NMR (200 MHz, CD₃CN): δ0.61 (m, 2H), 0.81 (m, 2H), 0.85 (s, 3H), 2.83 (m, 1H), 3.74-3.86 (m, 1H), 3.93-4.03 (m, 2H), 4.11 (d, J=8.9 Hz, 1H), 6.02 (s, 1H), 6.49 (d, J=3.7 Hz, 1H), 7.00 (d, J=3.7 Hz, 1H).

### Example 120

3′,5′-Bis-[O-(1-oxooctyl )-2′-O-methylcytidine

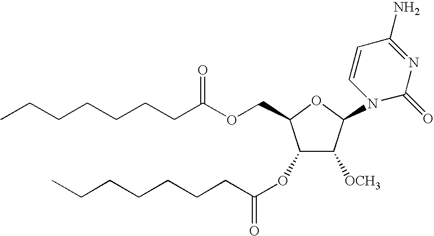

1,3-Dicyclohexylcarbodiimide (21.48 g, 104 mmol) was dissolved in anhydrous dichloromethane (100 mL). To the solution was added octanoic acid (5.49 mL, 34.5 mmol, made anhydrous by keeping over molecular sieves, 4 A° overnight at room temperature), and the resulting reaction mixture was stirred under argon atmosphere for 6 h. The white precipitate which formed was filtered, and the filtrate was concentrated under reduced pressure. The residue obtained was dissolved in anhydrous pyridine and added to N⁴-(4,4′-dimethoxytrityl)-2′-O-methylcytidine (0.43 g, 0.77). DMAP (0.09 g, 0.77 mmol) was added and the resulting mixture was stirred at room temperature under argon atmosphere for 12 h. The solvent was removed under reduced pressure and the residue obtained was dissolved in ethyl acetate (100 mL). The organic phase was washed with aqueous sodium bicarbonate (5%, 50 mL), dried over anhydrous Na₂SO₄ and concentrated under reduced pressure. The residue was purified by flash silica gel column chromatography and eluted with 5% MeOH in dichloromethane. The product obtained was dissolved in a mixture of acetic acid: MeOH: H₂O (20 mL, 3:6:1). The resulting mixture was heated at 50° C. for 24 h. The solvent was removed under reduced pressure. The residue obtained was purified by flash silica gel column chromatography and eluted with dichloromethane containing 0 to 5% of MeOH to give the title compound (0.22 g).

¹H NMR (200 MHz, DMSO-d₆) δ0.83 (m, 6H), 1.23 (br s, 16H), 1.51 (m, 4H), 2.33 (m, 4H), 3.26 (s, 3H), 4.06 (t, J=5.2 Hz, 1H), 4.21 (m, 3H), 5.11 (t, J=5.2Hz, 1H), 5.75 (d, J=7.4 Hz, 1H), 5.84 (d, J=4.8 Hz, 1H), 7.26 (br s, 2H), 7.61 (d, J=7.4Hz, 1H).

MS (ES): m/z 510.3 [M+H]⁺; HRMS (FAB) Calcd for C₂₆H₄₄N₃O₇: 510.3179; found 510.3170.

### Example 121

4-Amino-1- [symbol] -D-ribofuranosyl)-1H-pyrazolo[3,4-d]pyrimidine

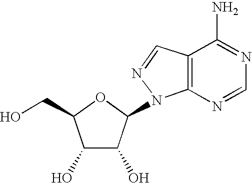

This compound was prepared following procedures described in Nucleic Acids Res., 11: 871-872 (1983).

### Example 122

2′C-Methyl-cytidine

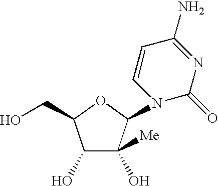

This compound was prepared following procedures described in L. Beigelman et al., Carbohyd. Res. 166: 219-232 (1987) or X-Q Tang, et al., J. Org. Chem. 64: 747-754 (1999).

### Example 123

4-Amino-7-(2-C-methyl-β-D-ribofuranosyl)-7H-pyrrolo[2,3-d]pyrimidine-5-carbonitrile

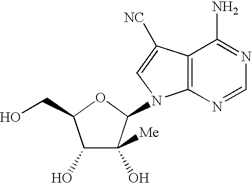

This compound was prepared following procedures described by Y. Murai et al. in Heterocycles 33: 391-404 (1992).

### Example 124

4-Amino-7-(2-C-methyl-β-D-ribofuranosyl)-7H-pyrrolo2,3-d]pyrimidine-5-carboxamide

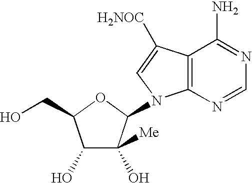

This compound was prepared following procedures described by Y. Murai et al. in Heterocycles 33: 391-404 (1992).

### Example 125

8-Aminoadenosine

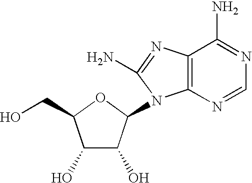

This compound was prepared following the procedure described in M. Ikehara and S. Yamada, Chem. Pharm. Bull., 19: 104 (1971).

### Example 130

7-(2-C-methyl-β-D-ribofuranosyl)-7H-pyrrolo[2,3-d]pyrimdin-4(3H)-one

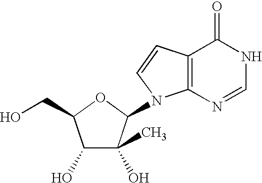

To the compound from Step E of Example 62 (59 mg, 0. 18 mmol) was added aqueous sodium hydroxide (1M). The mixture was heated to reflux for 1 hr, cooled, neutralized with aqueous HCl (2M) and evaporated in vacuo. The residue was purified on silica gel using dichloromethane/methanol (4:1) as eluent. Fractions containing the product were pooled and evaporated in vacuo to give the desired product (53 mg) as a colorless oil.

¹H NMR (acetonitrile-d₃): δ0.70 (s, 3H), 3.34-4.15 (overlapping m, 7H), 6.16 (s, 1H), 6.57 (d, 3.6 Hz, 1H), 7.37 (d, 3.6 Hz, 1H), 8.83 (s, 1H).

### Example 131

4-Amino-5-chloro-7-(2-C-methyl-β-D-ribofuranosyl)-7H-pyrrolo[2,3-d]pyrimidine

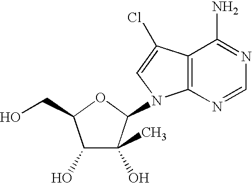

To a pre-cooled solution (0° C.) of the compound from Step F of Example 62 (140 mg, 0.50 mmol) in DMF (2.5 mL) was added N-chlorosuccinimide (0.075 g, 0.55 mmol) in DMF (0.5 mL) dropwise. The solution was stirred at rt for 1 h and the reaction quenched by addition of methanol (4 mL) and evaporated in vacuo. The crude product was purified on silica gel using methanol/dichloromethane (1:9) as cluent. Fractions containing the product were pooled and evaporated in vacuo to give the desired product (55 mg) as a colorless solid.

¹H NMR (acetonitrile-d₃): δ0.80 (s, 3H), 3.65-4.14 (overlapping m, 7H), 5.97 (s br, 2H), 6.17 (s, 1H), 7.51 (s, 1H), 8.16 (s, 1H).

ES-MS: Found: 315.0 (M+H⁺), calc.for C₁₂H₁₅ClN₄O₄+H⁺: 315.09.

### Example 132

4-Amino-5-bromo-7-(2-C-methyl-β-D-ribofuranosyl)-7H-pyrrolo[2,3-d]pyrimidine

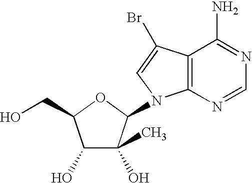

To a pre-cooled solution (0° C.) of the compound from Step F of Example 62 (28 mg, 0.10 mmol) in DMF (0.5 mL) was added N-bromosuccinimide (0.018 g, 0.10 mmol) in DMF (0.5 mL) dropwise. The solution was stirred at 0° C. for 20 min, then at rt for 10 min. The reaction was quenched by addition of methanol (4 mL) and evaporated in vacuo. The crude product was purified on silica gel using methanol/dichloromethane (1:9) as eluent. Fractions containing the product were pooled and evaporated in vacuo to give the desired product (13.0 mg) as a colorless solid.

¹H NMR (acetonitrile-d₃): δ0.69 (s, 3H), 3.46-4.00 (overlapping m, 7H), 5.83 (s br, 2H), 6.06 (s, 1H), 7.45 (s, 1H), 8.05 (s, 1H).

ES-MS: Found: 359.1 (M+H⁺), calc.for C₁₂H₁₅BrN₄O₄+H⁺: 359.04.

### Example 133

2-Amino-7-(2-C-methyl-β-D-ribofuranosyl)-7H-pyrrolo[2,3-d]pyrimidine

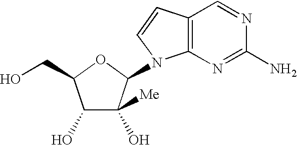

A mixture of 2-amino4-chloro-7-(2-C-methyl-β-D-ribofuranosyl)-7H-pyrrolo[2,3-d]pyrimidine (Example 118, Step B) (20 mg, 0.07 mmol) in EtOH (1.0 mL), pyridine (0.1 mL) and 10% Pd/C (6 mg) under H₂ (atmospheric pressure) was stirred overnight at room temperature. The mixture was filtered through a Celite pad which was thoroughly washed with EtOH. The combined filtrate was evaporated and purified on a silica gel column with CH₂Cl₂/MeOH, 20/1 and 10/1, as eluent to give the title compound as a white solid (16 mg).

¹H NMR (200 MHz, CD₃OD): δ0.86 (s, 3H, 2′C-Me), 3.82 (dd, J_{5′4′}=3.6 Hz, J_{5′,5″}=12.7 Hz, 1H, H-5′), 3.94-4.03 (m, 2H, H-5′, H-4′), 4.10 (d, J_{3′4′}=8.8 Hz, 1H, H-3′), 6.02 (s, 1H, H-1′), 6.41 (d, J_{5,6}=3.8 Hz, 1H, H-5), 7.39 (d, 1H, H-6), 8.43 (s, 1H, H-4). ES MS: 281.4 (MH⁺).

### Example 134

2-Amino-5-methyl-7-(2-C,2-O-dimethyl-β-D-ribofuranosyl)-7H-pyrrolo[2,3-d]pyrimidin-4(3H)-one

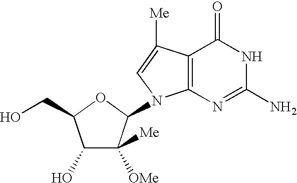

Step A: 2-Amino-4-chloro-7-[3,5-bis-O-(2,4-dichlorophenylmethyl)-2-C-methyl-β-D-ribofuranosyl]-5-methyl-7H-pyrrolo[2,3-d]pyrimidine

To an ice-cold solution of the product from Step C of Example 62 (1.57 g, 3.16 mmol) in CH₂Cl₂ (50 mL) was added HBr (5.7 M in acetic acid; 3.3 mL) dropwise. The reaction mixture was stirred at 0° C. for 1 h and then at room temperature for 2 h, concentrated in vacuo and co-evaporated with toluene (2×20 mL). The resulting oil was dissolved in MeCN (20 mL) and added dropwise to a solution of the sodium salt of 2-amino4-chloro-5-methyl-1H-pyrrolo[2,3-d]pyrimidine in acetonitrile [generated in situ from 2-amino-4-chloro-5-methyl-1H-pyrrolo[2,3-d]pyrimidine [for preparation, see Liebigs Ann. Chem. 1984: 708-721] (1.13 g, 6.2 mmol) in anhydrous acetonitrile (150 mL), and NaH (60% in mineral oil, 248 mg, 6.2 mmol), after 2 h of vigorous stirring at rt]. The combined mixture was stirred at rt for 1 day and then evaporated to dryness. The residue was suspended in water (100 mL) and extracted with EtOAc (300+150 mL). The combined extracts were washed with brine (100 mL), dried over Na₂SO₄, filtered and evaporated. The crude product was purified on a silica gel column (5×7 cm) using ethyl acetate/hexane (0 to 30% EtOAc in 5% step gradient) as the eluent. Fractions containing the product were combined and evaporated in vacuo to give the desired product (0.96 g) as a colorless foam.

Step B: 2-Amino-4-chloro-7-[3,5-bis-O-(2,4-dichlorophenylmethyl)-2-C,2-O-dimethyl-β-D-ribofuranosyl]-5-methyl-7H-pyrrolo[2,3-d]pyrimidine

To an ice-cold mixture of the product from Step A (475 mg, 0.7 mmol) in THF (7 mL) was added NaH (60% in mineral oil, 29 mg) and stirred at 0° C. for 0.5 h. Then Mel (48 μL) was added and reaction mixture stirred at rt for 1 day. The reaction was quenched with MeOH and the mixture evaporated. The crude product was purified on a silica gel column (5×3.5 cm) using hexane/ethyl acetate (9/1, 7/1, 5/1 and 3/1) as eluent. Fractions containing the product were combined and evaporated to give the desired compound (200 mg) as a colorless foam.

Step C: 2-Amino-7-[3,5-bis-O-(2,4-dichlorophenylmethyl)-2-C,2-O-dimethyl-β-D-ribofuranosyl]-5-methyl-7H-pyrrolo[2,3-d]pyrimidine-4(3H)-one

A mixture of the product from Step B (200 mg, 0.3 mmol) in 1,4-dioxane (15 mL) and aqueous NaOH (2N, 15 mL) in a pressure bottle was heated overnight at 135° C. The mixture was then cooled to 0° C., neutralized with 2N aqueous HCl and evaporated to dryness. The crude product was suspended in MeOH, filtered, and the solid thoroughly washed with MeOH. The combined filtrate was concentrated, and the residue purified on a silica gel column (5×5 cm) using CH₂Cl₂/MeOH (40/1, 30/1 and 20/1) as eluent to give the desired compound (150 mg) as a colorless foam.

Step D: 2-Amino-5-methyl-7-(2-C,2-O-dimethyl-β-D-ribofuranosyl)-7H-pyrrolo[2,3-d]pyrimidin-4(3H)-one

A mixture of the product from Step C (64 mg, 0.1 mmol) in MeOH (5 mL) and Et₃N (0.2 mL) and 10% Pd/C (24 mg) was hydrogenated on a Parr hydrogenator at 50 psi at r.t. for 1.5 days, then filtered through a Celite pad which was thoroughly washed with MeOH. The combined filtrate was evaporated and the residue purified on a silica gel column (3×4 cm) with CH₂Cl₂/MeOH (30/1, 20/1) as eluent to yield 2-amino-5-methyl-7-(5-O-benzyl-2-C,2-O-dimethyl-β-D-ribofuranosyl)-7H-pyrrolo[2,3-d]pyrimdin-4(3H)-one. The compound (37 mg) was further hydrogenated in EtOH (2 mL) with 10% Pd/C and under atmospheric pressure of hydrogen. After stirring 2 days at r.t., the reaction mixture was filtered through Celite, the filtrate evaporated and the crude product purified on a silica gel column (1×7 cm) with CH₂Cl₂/MeOH (30/1, 20/1 and 10/1) as eluent to yield the title compound (12 mg) after freeze-drying.

¹H NMR (200 MHz, CD₃OD): δ0.81 (s, 3H, 2′C-Me), 2.16 (d, J_{H-6,C5-Me}=1.3 Hz, 3H, C5-Me), 3.41 (s, 3H, 2′-OMe), 3.67 (dd, J_{5′4′}=3.4 Hz, J_{5′,5″}=12.6 Hz, 1H, H-5′), 3.81-3.91 (m, 3H, H-5″, H-4′, H-3′), 6.10 (s, 1H, H-1′), 6.66 (d, 1H, H-6).

ES MS: 323.3 (M−H)⁺.

### Example 135

4-Amino-5-methyl-7-(2-C-methyl-β-D-ribofuranosyl)-7H-pyrrolo[2,3-d]pyrimidine

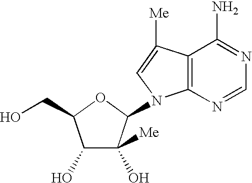

Step A: 4-Chloro-7-[3,5-bis-O-(2,4-dichlorophenylmethyl)-2-C-methyl-β-D-ribofuranosyl]-5-methyl-7H-pyrrolo[2,3-d]pyrimidine

To an ice-cold solution of the product from Step C of Example 62 (1.06 g, 2.1 mmol) in CH₂Cl₂ (30 mL) was added HBr (5.7 M in acetic acid; 2.2 mL) dropwise. The reaction mixture was stirred at 0° C. for 1 h and then at room temperature for 2 h, concentrated in vacuo and co-evaporated with toluene (2×15 mL). The resulting oil was dissolved in MeCN (10 mL) and added dropwise into a solution of the sodium salt of 4-chloro-5-methyl-1H-pyrrolo[2,3-d]pyrimidine in acetonitrile [generated in situ from 4-chloro-5-methyl-1H-pyrrolo[2,3-d]pyrimidine [for preparation, see J. Med. Chem. 33: 1984 (1990)] (0.62 g, 3.7 mmol) in anhydrous acetonitrile (70 mL), and NaH (60% in mineral oil, 148 mg, 3.7 mmol), after 2 h of vigorous stirring at rt]. The combined mixture was stirred at rt for 1 day and then evaporated to dryness. The residue was suspended in water (100 mL) and extracted with EtOAc (250+100 mL). The combined extracts were washed with brine (50 mL), dried over Na₂SO₄, filtered and evaporated. The crude product was purified on a silica gel column (5×5 cm) using hexane/ethyl acetate (9/1, 5/1, 3/1) gradient as the eluent. Fractions containing the product were combined and evaporated in vacuo to give the desired product (0.87 g) as a colorless foam.

Step B: 4-Chloro-5-methyl-7-(2-C-methyl-β-D-ribofuranosyl)-7H-pyrrolo2,3-d]pyrimidine

To a solution of the compound from Step A (0.87 g, 0.9 mmol) in dichloromethane (30 mL) at −78° C. was added boron trichloride (1M in dichloromethane, 9.0 mL, 9.0 mmol) dropwise. The mixture was stirred at −78° C. for 2.5 h, then at −30° C. to −20° C. for 3 h. The reaction was quenched by addition of methanol/dichloromethane (1:1) (9 mL) and the resulting mixture stirred at −15° C. for 30 min., then neutralized with aqueous ammonia at 0° C. and stirred at rt for 15 min. The solid was filtered and washed with CH₂Cl₂/MeOH (1/1, 50 mL). The combined filtrate was evaporated, and the residue was purified on a silica gel column (5×5 cm) using CH₂Cl₂ and CH₂Cl₂/MeOH (40/1 and 30/1) gradient as the eluent to furnish the desired compound (0.22 g) as a colorless foam.

Step C: 4-Amino-5-methyl-7-(2-C-methyl-β-D-ribofuranosyl)-7H-pyrrolo[2,3-d]pyrimidine

To the compound from Step B (0.2 g, 0.64 mmol) was added methanolic ammonia (saturated at 0° C.; 40 mL). The mixture was heated in a stainless steel autoclave at 100° C. for 14 h, then cooled and evaporated in vacuo. The crude mixture was purified on a silica gel column (5×5 cm) with CH₂Cl₂/MeOH (50/1, 30/1, 20/1) gradient as eluent to give the title compound as a white solid (0.12 g).

¹H NMR (DMSO-d₆): δ0.60 (s, 3H, 2′C-Me), 2.26 (s, 3H, 5C-Me), 3.52-3.61 (m, 1H, H-5′), 3.70-3.88 (m, 3H, H-5″, H-4′, H-3′), 5.00 (s, 1H, 2′-OH), 4.91-4.99 (m, 3H, 2′-OH, 3′-OH, 5′-OH), 6.04 (s, 1H, H-1′), 6.48 (br s, 2H, NH₂), 7.12 (s, 1H, H-6), 7.94 (s, 1H, H-2). ES MS: 295.2 (MH⁺).

### Example 136

4-Amino-7-(2-C-methyl-β-D-ribofuranosyl)-7H-pyrrolo[2,3-d]pyrimidine-5-carboxylic acid

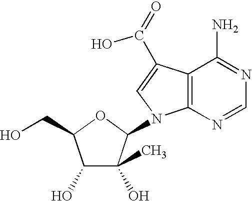

The compound of Example 123 (0.035 g, 0.11 mmol) was dissolved in a mixture of aqueous ammonia (4 mL, 30 wt %) and saturated methanolic ammonia (2 mL), and a solution of H₂O₂ in water (2 mL, 35 wt %) was added. The reaction mixture was stirred at room temperature for 18 h. Solvent was removed under reduced pressure, and the residue obtained was purified by HPLC on a reverse phase column (Altech Altima C-18, 10×299 mm, A=water, B=acetonitrile, 10 to 60% B in 50 min, flow 2 mL/min) to yield the title compound (0.015 g, 41%) as a white solid.

¹H NMR (CD₃OD): δ0.85 (s, 3H, Me), 3.61 (m, 1H), 3.82 (m, 1H) 3.99-4.86 (m, 2H), 6.26 (s, 1H), 8.10 (s, 2H) 8.22(s, 1H); ¹³C NMR (CD₃OD): 20.13, 61.37, 73.79, 80.42, 84.01, 93.00, 102.66, 112.07, 130.07, 151.40, 152.74, 159.12, 169.30.

HRMS (FAB) Calcd for C₁₃H₁₇N₄O₆⁺ 325.1148, found 325.1143.

### Example 137

4-Amino-7-(2-C-vinyl-(β-D-ribofuranosyl)-7H-pyrrolo[2,3-d]pyrimidine

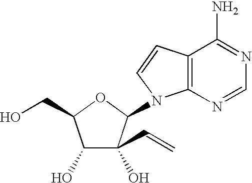

Step A: 3,5-Bis-O-(2,4-dichlorophenylmethyl)-2-C-vinyl-1-O-methyl-α-D-ribofuranose

Cerium chloride heptahydrate (50 g, 134.2 mmol) was finely crushed in a pre-heated mortar and transferred to a round-bottom flask equipped with a mechanical stirrer. The flask was heated under high vacuum overnight at 160° C. The vacuum was released under argon and the flask was cooled to room temperature. Anhydrous THF (300 mL) was cannulated into the flask. The resulting suspension was stirred at room temperature for 4 h and then cooled to −78° C. Vinylmagnesium bromide (1M in THF, 120 mL, 120 mmol) was added and stirring continued at −78° C. for 2 h. To this suspension was added a solution of 3,5-bis-O-(2,4-dichlorophenylmethyl)-1-O-methyl-α-D-erythro-pentofuranose-2-ulose (14 g, 30 mmol) [from Example 2, Step B] in anhydrous THF (100 mL), dropwise with constant stirring. The reaction was stirred at −78° C. for 4 h. The reaction was quenched with sat. ammonium chloride solution and allowed to come to room temperature. The mixture was filtered through a celite pad and the residue washed with Et₂O (2×500 mL). The organic layer was separated and the aqueous layer extracted with Et₂O (2×200 mL). The combined organic layers were dried over anhydrous Na₂SO₄ and concentrated to a viscous yellow oil. The oil was purified by flash chromatography (SiO₂, 10% EtOAc in hexanes). The title compound (6.7 g, 13.2 mmol) was obtained as a pale yellow oil.

Step B: 4-Chloro-7-[3,5-bis-O-(2,4-dichlorophenylmethyl)-2-C-vinyl-β-D-ribofuranosyl]-7H-pyrrolo[2,3-d]pyrimidine

To a solution of the compound from Step A (6.4 g, 12.6 mmol) in anhydrous dichloromethane (150 mL) at −20° C. was added HBr (30% solution in AcOH, 20 mL, 75.6 mmol) dropwise. The resulting solution was stirred between −10° C. and 0° C. for 4 h, evaporated in vacuo and co-evaporated with anhydrous toluene (3×40 mL). The oily residue was dissolved in anhydrous acetonitrile (100 mL) and added to a solution of the sodium salt of 4-chloro-1H-pyrrolo[2,3-d]pyrimidine (5.8 g, 37.8 mmol) in acetonitrile (generated in situ as described in Example 62) at −20° C. The resulting mixture was allowed to come to room temperature and stirred at room temperature for 1 day. The mixture was then evaporated to dryness, taken up in water and extracted with EtOAc (2×300 mL). The combined extracts were dried over Na₂SO₄, filtered and evaporated. The crude mixture was purified by flash chromatography (SiO₂, 10% EtOAc in hexanes) and the title compound (1.75 g) isolated as a white foam.

Step C: 4-Amino-7-[3,5-bis-O-(2,4-dichlorophenylmethvl)-2-C-vinyl-β-D-ribofuranosyl]-7H-pyrrolo[2,3-d]pyrimidine

The compound from Step B (80, mg) was dissolved in the minimum amount of 1,4-dioxane and placed in a stainless steel bomb. The bomb was cooled to −78° C. and liquid ammonia was added. The bomb was sealed and heated at 90° C. for 1 day. The ammonia was allowed to evaporate and the residue concentrated to a white solid which was used in the next step without further purification.

Step D: 4-Amino-7-(2-C-vinyl-β-D-ribofuranosyl)-7H-pyrrolo[2,3-d]pyrimidine

To a solution of the compound from Step C (60 mg) in dichloromethane at −78° C. was added boron trichloride (1M in dichloromethane) dropwise. The mixture was stirred at −78° C. for 2.5 h, then at −30° C. to −20° C. for 3 h. The reaction was quenched by addition of methanol/dichloromethane (1:1) and the resulting mixture stirred at −15° C. for 0.5 h, then neutralized with aqueous ammonia at 0° C. and stirred at room temperature for 15 min. The solid was filtered and washed with methanol/dichloromethane (1:1). The combined filtrate was evaporated and the residue purified by flash chromatography (SiO₂, 10% methanol in EtOAc containing 0.1% triethylamine). The fractions containing the product were evaporated to give the title compound as a white solid (10 mg).

¹H NMR (DMSO-d₆): δ3.6 (m, 1H, H-5′), 3.8 (m, 1H, H-5″), 3.9 (m d, 1-H, H-4′), 4.3 (t, 1H, H-3′), 4.8-5.3(m, 6H, CH═CH₂, 2′-OH, 3′-OH, 5′-OH) 6.12 (s, 1H, H-1). 6.59 (d, 1H, H-5), 7.1 (br s, 1H, NH2), 7.43 (d, 1H, H-6), 8.01 (s, 1H, H-2). ES-MS: Found: 291.1 (M−H⁻); calc. for C₁₃H₁₆N₄O₄−H⁻: 291.2.

### Example 138

4-Amino-7-(2-C-hydroxymethyl-β-D-ribofuranosyl)-7H-pyrrolo[2,3-d]pyrimidine

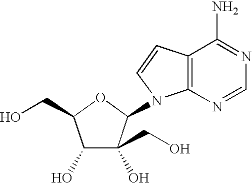

Step A: 4-Chloro-7-[3,5-bis-O-(2,4-dichlorophenylmethyl)-2-C-hydroxymethyl-β-D-ribofuranosyl]-7H-pyrrolo[2,3-d]pyrimidine

To a solution of the compound from Example 137, Step B (300 mg, 0.48 mmol) in 1,4-dioxane (5 mL) were added N-methylmorpholine-N-oxide (300 mg, 2.56 mmol) and osmium tetroxide (4% solution in water, 0.3 mL). The mixture was stirred in the dark for 14 h. The precipitate was removed by filtration through a celite plug, diluted with water (3×), and extracted with EtOAc. The EtOAc layer was dried over Na₂SO₄ and concentrated in vacuo. The oily residue was taken up in dichloromethane (5 mL) and stirred over NaIO₄ on silica gel (3 g, 10% NaIO₄) for 12 h. The silica gel was removed by filtration and the residue was evaporated and taken up in absolute ethanol (5 mL). The solution was cooled in an ice bath and sodium borohydride (300 mg, 8 mmol) was added in small portions. The resulting mixture was stirred at room temperature for 4 h and then diluted with EtOAc. The organic layer was washed with water (2×20 mL), brine (20 mL) and dried over Na₂SO₄. The solvent was evaporated and the residue purified by flash chromatography (SiO₂, 2:1 hexanes/EtOAc) to give the title compound (160 mg, 0.25 mmol) as white flakes.

Step B: 4-Amino-7-[3,5-bis-O-(2,4-dichlorophenylmethyl)-2-C-hydroxymethyl-β-D-ribofuranosyl]-7H-pyrrolo[2,3-d]pyrimidine

The compound from Step A (150 mg, 0.23 mmol) was dissolved in the minimum amount of 1,4-dioxane (10 mL) and placed in a stainless steel bomb. The bomb was cooled to −78° C. and liquid ammonia was added. The bomb was sealed and heated at 90° C. for 1 day. The ammonia was allowed to evaporate and the residue concentrated to a white solid which was used in the next step without further purification.

Step C: 4-Amino-7-(2-C-hydroxymethyl-β-D-ribofuranosyl)-7H-pyrrolo[2,3-d]pyrimidine

The compound from Step B (120 mg, 0.2 mmol) was dissolved in 1:1 methanol/dichloromethane, 10% Pd-C was added, and the suspension stirred under an H₂ atmosphere for 12 h. The catalyst was removed by filtration through a celite pad and washed with copious amounts of methanol. The combined filtrate was evaporated in vacuo and the residue was purified by flash chromatography (SiO₂, 10% methanol in EtOAc containing 0.1% triethylamine) to give the title compound (50 mg) as a white powder.

¹H NMR (CD₃OD): δ3.12 (d, 1H, CH₂′), 3.33 (d, 1H, CH₂″), 3.82 (m, 1H, H-5′), 3.99-4.1(m, 2H, H-4′, H-5″), 4.3 (d, 1H, H-3′), 6.2 (s, 1H, H-1′), 6.58 (d, 1H, H-5), 7.45 (d, 1H, H-6), 8.05 (s, 1H, H-2). LC-MS: Found: 297.2 (M+H⁺); calc. for C₁₂H₁₆N₄O₅+H⁺: 297.3.

### Example 139

4-Amino-7-(2-C-fluoromethyl-β-D-ribofuranosyl)-7H-pyrrolo[2.3-d]pyrimidine

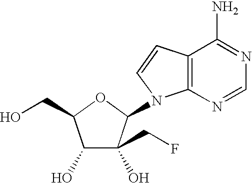

Step A: 4-Chloro-7-[3,5-bis-O-(2,4-dichlorophenylmethyl)-2-C-fluoromethyl-β-D-ribofuranosyl]-7H-pyrrolo[2,3-d]pyrimidine

To a solution of the compound from Example 138, Step A (63 mg, 0.1 mmol) in anhydrous dichloromethane (5 mL) under argon, were added 4-dimethylaminopyridine (DMAP) (2 mg, 0.015 mmol) and triethylamine (62 μL, 0.45 mmol). The solution was cooled in an ice bath and p-toluenesulfonyl chloride (30 mg, 0.15 mmol) was added. The reaction was stirred at room temperature overnight, washed with NaHCO₃ (2×10 mL), water (10 mL), brine (10 mL), dried over Na₂SO₄ and concentrated to a pink solid in vacuo. The solid was dissolved in anhydrous THF (5 mL) and cooled in an icebath. Tetrabutylammonium fluoride (1M solution in THF, 1 mL, 1 mmol) was added and the mixture stirred at room temperature for 4 h. The solvent was removed in vacuo, the residue taken up in dichloromethane, and washed with NaHCO₃ (2×10 mL), water (10 mL) and brine (10 mL). The dichloromethane layer was dried over anhydrous Na₂SO₄, concentrated in vacuo, and purified by flash chromatography (SiO₂, 2:1 hexanes/EtOAc) to afford the title compound (20 mg) as a white solid.

Step B: 4-Amino-7-[3,5-bis-O-(2,4-dichlorophenylmethyl)-2-C-fluoromethyl-β-D-ribofuranosyl]-7H-pyrrolo[2,3-d]pyrimidine

The compound from Step A (18 mg, 0.03 mmol) was dissolved in the minimum amount of 1,4-dioxane and placed in a stainless steel bomb. The bomb was cooled to −78° C. and liquid ammonia was added. The bomb was sealed and heated at 90° C. for 1 day. The ammonia was allowed to evaporate and the residue concentrated to a white solid which was used in the next step without further purification.

Step C: 4-Amino-7-(2-C-fluoromethyl-β-D-ribofuranosyl)-7H-pyrrolo[2,3-d]pyrimidine

The compound from Step B (16 mg) was dissolved in 1:1 methanol/dichloromethane, 10% Pd-C was added, and the suspension stirred under an H₂ atmosphere for 12 h. The catalyst was removed by filtration through a celite pad and washed with copious amounts of methanol. The combined filtrate was evaporated in vacuo and the residue was purified by flash chromatography (SiO₂, 10% methanol in EtOAc containing 0.1% triethylamine) to give the title compound (8 mg) as a white powder.

¹H NMR (DMSO-d₆): δ3.6-3.7 (m, 1H, H-5′), 3.8-4.3 (m, 5H, H-5″, H-4′, H-3′, CH₂) 5.12 (t, 1H, 5′-OH), 5.35 (d, 1H, 3′-OH), 5.48 (s, 1H, 2′-OH), 6.21 (s, 1H, H-1′), 6.52 (d, 1H, H-5), 6.98 (br s, 2H, NH2), 7.44 (d, 1H, H-6), 8.02 (s, 1H, H-2). ¹⁹F NMR (DMSO-d₆): δ-230.2 (t).

ES-MS: Found: 299.1 (M+H⁺), calc.for C₁₂H₁₅FN₄O₄+H⁺: 299.27.

### Examples 140 and 141

7-(3-Deoxy-2-C-methyl-β-D-ribofuranosyl)-7H-pyrrolo[2,3-d]pyrimidine and 7-(3-deoxy-2-C-methyl-β-D-arabinofuranosyl)-7H-pyrrolo[2,3-d]pyrimidine

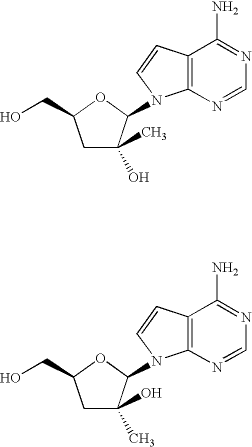

Step A: 7-[2,5-Bis-O-(tert-butyldimethylsilyl)-β-D-ribofuranosyl]-7H-pyrrolo[2,3-d]pyrimidine and 7-[3,5-Bis-O-(tert-butyldimethylsilyl)-β-D-ribofuranosyl]-7H-pyrrolo[2,3-d]pyrimidine

To a stirred solution of tubercidin (5.0 g, 18.7 mmol) in a mixture of pyridine (7.5 mL) and DMF (18.5 mL) was added silver nitrate (6.36 g, 38.8 mmol). This mixture was stirred at room temperature for 2 h. It was cooled in an ice bath and THF (37.4 mL) and tert-butyldimethylsilyl chloride (5.6 g, 37 mmol) was added and the mixture was stirred at room temperature for 2 h. The mixture was then filtered through a pad of celite and washed with THF. The filtrate and washings were diluted with ether containing a small amount of chloroform. The organic layer was washed successively with sodium bicarbonate and water (3×50 mL), dried over anhydrous sodium sulfate and concentrated. The pyridine was removed by coevaporation with toluene and the residue was purified by flash chromatography on silica gel using 5-7% MeOH in CH₂Cl₂ as the eluent; yield 3.0 g.

Step B: 7-[2,5-Bis-O-(tert-butyldimethylsilyl)-β-D-ribofuranosyl)]-4-[di-(4-methoxyphenyl)phenylmethyl]amino-7H-pyrrolo[2,3-d]pyrimidine and 7-[3,5-bis-O-(tert-butyldimethylsilyl)-β-D-ribofuranosyl]-4-[di-(4-methoxyphenyl)phenylmethyl]amino-7H-pyrrolo[2,3-d]pyrimidine

To a solution of mixture of the compounds from Step A (3.0 g, 6.0 mmol) in anhydrous pyridine (30 mL) was added 4,4′-dimethoxytrityl chloride (2.8 g, 8.2 mmol) and the reaction mixture was stirred at room temperature overnight. The mixture was then triturated with aqueous pyridine and extracted with ether. The organic layer was washed with water, dried over anhydrous sodium sulfate and concentrated to a yellow foam (5.6 g). The residue was purified by flash chromatography over silica gel using 20-25% EtOAc in hexanes as the eluent. The appropriate fractions were collected and concentrated to furnish 2′,5′-O-bis-O-(tert-butyldimethylsilyl)-and 3′,5′-bis-O-(tert-butyldimethylsilyl) protected nucleosides as colorless foams (2.2 g and 1.0 g, respectively).

Step C: 7-[2,5-Bis-O-(tert-butyldimethylsilyl)-3-O-tosyl-β-D-ribofuranosyl)]-4-[di-(4-methoxyphenol)phenylmethyl]amino-7H-pyrrolo[2,3-d]pyrimidine

To an ice-cooled solution of 2′,5′-bis-O-(tert-butyldimethylsilyl)-protected nucleoside from Step B (2.0 g, 2.5 mmol) in pyridine (22 mL) was added p-toluenesulfonyl chloride (1.9 g, 9.8 mmol). The reaction mixture was stirred at room temperature for four days. It was then triturated with aqueous pyridine (50%, 10 mL) and extracted with ether (3×50 mL) containing a small amount of CH₂Cl₂ (10 mL). The organic layer was washed with sodium bicarbonate and water (3×30 mL). The organic layer was dried over anhydrous Na₂SO₄ and concentrated. Pyridine was removed by co-evaporation with toluene (3×25 mL). The residual oil was filtered through a pad of silica gel using hexane:ethyl acetate (70:30) as eluent; yield 1.4 g.

Step D: 4-[di-(4-methoxyphenyl)phenylmethyl]amino-7-[3-O-tosyl-β-D-ribofuranosyl-7H-pyrrolo[2,3-d]pyrimidine

A solution of the compound from Step C (1.0 g, 1.1 mmol) and THF (10 mL) was stirred with tetrabutylammonium fluoride (1M solution in THF, 2.5 mL) for 0.5 h. The mixture was cooled and diluted with ether (50 mL). The solution was washed with water (3×50 mL), dried over anhydrous Na₂SO₄ and concentrated to an oil. The residue was purified by passing through a pad of silica gel using hexane: ethyl acetate (1:1) as eluent; yield 780 mg.

Step E: 7-(3-Deoxy-2-C-methyl-β-D-ribofuranosyl)-7H-pyrrolo[2,3-d]-pyrimidine and 7-(3-Deoxy-2-C-methyl-β-D-arabinofuranosyl)-7H-pyrrolo-[2,3-d]pyrimidine

A solution of CH₃MgI (3.0 M solution in ether, 3.0 mL) in anhydrous toluene (3.75 mL) was cooled in an ice bath. To this was added a solution of the compound from Step D (500 mg, 0.8 mmol) in anhydrous toluene (3.7 mL). The resulting mixture was stirred at room temperature for 3.5 h. It was cooled and treated with aqueous NH₄Cl solution and extracted with ether (50 mL containing 10 mL of CH₂Cl₂). The organic layer was separated and washed with brine (2×30 mL) and water (2×25 mL), dried over anhydrous Na₂SO₄ and concentrated to an oil which was purified by flash chromatography on silica gel using 4% MeOH in CH₂Cl₂ to furnish the 2-C-α-methyl compound (149 mg) and the 2-C-β-methyl compound (34 mg). These derivatives were separately treated with 80% acetic acid and the reaction mixture stirred at room temperature for 2.5 h. The acetic acid was removed by repeated co-evaporation with ethanol and toluene. The residue was partitioned between chloroform and water. The aqueous layer was washed with chloroform and concentrated. The evaporated residue was purified on silica gel using 5-10% MeOH in CH₂Cl₂ as the eluent to furnish the desired compounds as white solids.

7-(3-Deoxy-2-C-methyl-β-D-ribofuranosyl)-7H-pyrrolo[2,3-d]pyrimidine (9.0 mg):

¹H NMR (DMSO-d₆): δ0.74 (s, 3H, CH₃), 1.77 (dd, 1H, H-3′), 2.08 (t, 1H, H-3″), 3.59 (m, 1H, H-5′), 3.73 (m, 1H, H-5″), 4.15 (m, 1H, H-4′), 5.02 (t, 1H, OH-5′), 5.33 (s, 1H, OH-2′), 6.00 (s, 1H, H-1′), 6.54 (d, 1H, H-7), 6.95 (br s, 2H, NH₂), 7.47 (d, 1H, H-8), 8.00 (s, 1H, H-2); ES-MS: 263.1 [M−H].

7-(3-Deoxy-2-C-methyl-β-D-arabinofuranosyl)-7H-pyrrolo[2,3-d]pyrimidine (15 mg):

¹H NMR (DMSO-d₆): δ1.23 (s, 3H, CH₃), 2.08 (ddd, 2H, H-3′ and 3″), 3.57 (m, 2H, H-5′ and 5″), 4.06 (m, 1H, H-4), 5.10 (s, 1H, OH-2′), 5.24 (t, 1H, OH-5′), 6.01 (s, 1H, H-1′), 6.49 (d, 1H, H-7),6.89 (br s, 2H, NH₂), 7.35 (d, 1H, H-8), 8.01 (s,1H,H-2). ES-MS: 265.2[M+H].

### Example 142

4-Amino-7-(2,4-C-dimethyl-β-D-ribofuranosyl)-7H-pyrrolo[2,3-d]pyrimidine

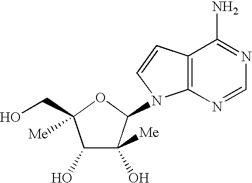

Step A: 5-Deoxy-1,2-O-isopropylidene-D-xylofuranose

1,2-O-Isopropylidene-D-xylofuranose (38.4 g, 0.2 mol), 4-dimethylaminopyridine (5 g), triethylamine (55.7 mL, 0.4 mol) were dissolved in dichloromethane (300 mL). p-Toluenesulfonyl chloride (38.13 g, 0.2 mol) was added and the reaction mixture was stirred at room temperature for 2 hours. The reaction mixture was then poured into saturated aqueous sodium bicarbonate (500 mL) and the two layers were separated. The organic layer was washed with aqueous citric acid solution (20%, 200 mL), dried (Na₂SO₄) and evaporated to give a solid (70.0 g). The solid was dissolved in dry THF (300 mL) and LiAlH₄ (16.0 g, 0.42 mol) was added in portions over 30 min. The mixture was stirred at room temperature for 15 hours. Ethyl acetate (100 mL) was added dropwise over 30 min and the mixture was filtered through a silica gel bed. The filtrate was concentrated and the resulting oil was chromatographed on silica gel (EtOAc/hexane 1/4) to afford the product as a solid (32.5 g).

Step B: 3,5-Bis-O-(2,4-dichlorophenylmethyl)-1-O-methyl-4-methyl-α-D-ribofuranose

Chromium oxide (50 g, 0.5 mol), acetic anhydride (50 mL, 0.53 mol) and pyridine (100 mL, 1.24 mol) were added to dichloromethane (1 L) in an ice water bath and the mixture was stirred for 15 min. 5-Deoxy-1,2-O-isopropylidene-D-xylofuranose (32 g, 0.18 mol) in dichloromethane (200 mL) was added, and the mixture was stirred at the same temperature for 30 min. The reaction solution was diluted with ethyl acetate (1 L) and filtered through a silica gel bed. The filtrate was concentrated to give a yellow oil. The oil was dissolved in 1,4-dioxane (1 L) and formaldehyde (37%, 200 mL). The solution was cooled to 0° C. and-solid KOH (50 g) was added. The mixture was stirred at room temperature overnight and was then extracted with ethyl acetate (6×200 mL). After concentration, the residue was chromatographed on silica gel (EtOAc) to afford the product as an oil (1.5 g). The oil was dissolved in 1-methyl-2-pyrrolidinone (20 mL) and 2,4-dichlorophenylmethyl chloride (4 g, 20.5 mmol) and NaH (60%, 0.8 g) were added. The mixture was stirred overnight and diluted with toluene (100 mL). The mixture was then washed with saturated aqueous sodium bicarbonate (3×50 mL), dried (Na₂SO₄) and evaporated. The residue was dissolved in methanol (50 m]L) and HCl in dioxane (4 M, 2 mL) was added. The solution was stirred overnight and evaporated. The residue was chromatographed on silica gel (EtOAc/hexane: 1/4) to afford the desired product as an oil (2.01 g).

Step C: 3,5-Bis-O-(2,4-dichlorophenylmethyl)-2,4-di-C-methyl-1-O-methyl-α-D-ribofuranose

The product (2.0 g, 4.0 mmol) from Step B and Dess-Martin periodinane (2.0 g) in dichloromethane (30 mL) were stirred overnight at room temperature and was then concentrated under reduced pressure. The residue was triturated with ether ether (50 mL) and filtered. The filtrate was washed with a solution of Na₂S₂O₃.5H₂O (2.5 g) in saturated aqueous sodium bicarbonate solution (50 mL), dried (MgSO₄), filtered and evaporated. The residue was dissolved in anhydrous Et₂O (20 mL) and was added dropwise to a solution of MeMgBr in Et₂O (3 M, 10 mL) at −78° C. The reaction mixture was allowed to warm to −30° C. and stirred at −30° C. to −15° C. for 5 h, then poured into saturated aqueous ammonium chloride (50 mL). The two layers were separated and the organic layer was dried (MgSO₄), filtered and concentrated. The residue was chromatographed on silica gel (EtOAc/hexane: 1/9) to afford the title compound as a syrup (1.40 g).

Step D: 4-Chloro-7-[3,5-bis-O-(2,4-dichlorophenylmethyl)-2,4-di-C-methyl-β-D-ribofuranosyl]-7H-pyrrolo[2,3-d]pyrimidine

To the compound from Step C (0.70 g, 1.3 mmol) was added HBr (5.7 M in acetic acid, 2 mL). The resulting solution was stirred at room temperature for 1 h, evaporated in vacuo and co-evaporated with anhydrous toluene (3×10 mL). 4-Chloro-1H-pyrrolo[2,3-d]pyrimidine (0.5 g, 3.3 mmol) and powdered KOH (85%, 150 mg, 2.3 mmol) were stirred in 1-methyl-2-pyrrolidinone (5 mL) for 30 min and the mixture was co-evaporated with toluene (10 mL). The resulting solution was poured into the above bromo sugar residue and the mixture was stirred overnight. The mixture was diluted with toluene (50 mL), washed with water (3×50 mL) and concentrated under reduced pressure. The residue was chromatographed on silica gel eluting with (EtOAc/ Hexane 15/85) to afford a solid (270 mg).

Step E: 4-Amino-7-(2,4-di-C-methyl-β-D-ribofuranosyl)-7H-pyrrolo[2,3-d]pyrimidine

The compound from Step D (270 mg) was dissolved in dioxane (2 mL) and liquid ammonia (20 g) was added in a stainless steel autoclave. The mixture was heated at 100° C. for 15 hours, then cooled and evaporated. The residue was chromatographed on silica gel (EtOAc) to afford a solid (200 mg). The solid (150 mg) and Pd/C (10% 150 mg) in methanol (20 mL) were shaken under H₂ (30 psi) for 3 h, filtered and evaporated. The residue was chromatographed on silica gel (MeOH/CH₂Cl₂: 1/9) to afford the desired product as a solid (35 mg).

¹H NMR (DMSO-d₆): δ0.65 (s, 3H), 1.18 (s, 3H), 3.43 (m, 2H), 4.06 (d, 1H, J 6.3 Hz), 4.87 (s, 1H), 5.26 (br, 1H), 5.08 (d, 1H, J 6.3 Hz), 5.25 (t, 1H, J 3.0 Hz), 6.17 (s, 1H), 6.54 (d, 1H, J 3.5 Hz), 6.97 (s, br, 2H), 7.54 (d, 1H, J 3.4 Hz), 8.02 (s, 1H). ¹³C NMR (DMSO-d₆): δ18.19, 21.32, 65.38, 73.00, 79.33, 84.80, 90.66, 99.09, 102.41, 12.90, 149.58, 151.48, 157.38. LC-MS: Found: 295.1 (M+H⁺); calculated for C₁₃H₁₈N₄_{O}₄+H⁺: 295.1

### Example 143

4-Amino-7-(3-deoxy-3-fluoro-2-C-methyl-β-D-ribofuranosyl)-7H-pyrrolo[2,3-d]pyrimidine

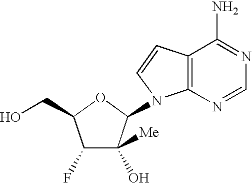

Step A: 3-Deoxy-3-fluoro-1-O-methyl-5-O-toluoyl-α-D-ribofuranose

1,2-O-Isopropylidene-D-xylofuranose (9.0 g, 50 mmol) and p-toluoyl chloride (7.0 mL, 50 mmol) in pyridine (50 mL) were stirred for 30 min. Water (10 mL) was added and the mixture was concentrated under reduced pressure. The residue was dissolved in toluene (500 mL) and the solution was washed with water (200 mL) and saturated aqueous sodium bicarbonate (200 mL). The two layers were separated and the organic layer was evaporated. The residue was dissolved in methanol (100 mL) and HCl in dioxane (4 M, 10 mL) was added. The mixture was stirred at room temperature overnight and was then evaporated under reduced pressure. The resulting oil was chromatographed on silica gel (EtOAc/hexane: 1/1) to afford an oil (10.1 g). The oil was dissolved in dichloromethane (100 mL) and diethylaminosulfur trifluoride (DAST) (5.7 mL) was added. The mixture was stirred overnight and was then poured into saturated aqueous sodium bicarbonate solution (100 mL). The mixture was extracted with toluene (2×50 mL) and the combined organic layers were concentrated. The residue was chromatographed on silica gel (EtOAc/hexane: 15/85) to afford the title compound as an oil (1.50 g).

Step B: 3-Deoxy-3-fluoro-2-C-methyl-1-O-methyl-α-O-toluoyl-α-D-ribofuranose

The product from Step A (1.0 g, 3.5 mmol) and Dess-Martin periodinane (2.5 g) in dichloromethane (20 mL) were stirred overnight at room temperature and was then concentrated under reduced pressure. The residue was triturated with diethyl ether (50 mL) and filtered. The filtrate was washed with a solution of Na₂S₂O₃. 5H₂O (12.5 g) in saturated aqueous sodium bicarbonate (100 mL), dried (MgSO₄), filtered and evaporated. The residue was dissolved in anhydrous THF (50 mL). TiCl₄ (3 mL) and methyl magnesium bromide in ethyl ether (3 M, 10 mL) were added at −78° C. and the mixture was stirred at −50 to −30° C. for 2 h. The mixture was poured into saturated aqueous sodium bicarbonate solution (100 mL) and filtered through Celite. The filtrate was extracted with toluene (100 mL) and evaporated. The residue was chromatographed on silica gel (EtOAc/hexane: 15/85) to afford the title compound as an oil (150 mg).

Step C: 4-Amino-7-(3-deoxy-3-fluoro-2-C-methyl-β-D-ribofuranosyl)-7H-pyrrolo[2,3-d]pyrimidine

The product from Step B (150 mg, 0.5 mmol) was dissolved in HBr (30%) in acetic acid (2 mL). After one hour, the mixture was evaporated under reduced pressure and co-evaporated with toluene (10 mL). 4-Chloro-1H-pyrrolo[2,3-d]pyrimidine (0.5 g, 3.3 mmol) and powdered KOH (85%, 150 mg, 2.3 mmol) were stirred in DMF (3 mL) for 30 min and the mixture was co-evaporated with toluene (2 mL). The resulting solution was poured into the above bromo sugar and the mixture was stirred overnight. The mixture was diluted with toluene (50 mL), washed with water (3×50 mL) and concentrated under reduced pressure. The residue was chromatographed on silica gel (EtOAc/hexane 15/85) to afford an oil (60 mg). The oil was dissolved in dioxane (2 mL) and liquid ammonia (20 g) was added in a stainless steel autoclave. The mixture was heated at 85° C. for 18 hours, then cooled and evaporated. The residue was chromatographed on silica gel (methanol/dichloromethane: 1/9) to afford the title compound as a solid (29 mg).

¹H NMR (DMSO-d₆): δ0.81 (s, 3H), 3.75 (m, 2H), 4.16 (m, 1H), 5.09 (dd, 1H, J 53.2, 7.8 Hz), 5.26 (br, 1H), 5.77 (s, 1H), 6.15 (d, 1H, J 2.9 Hz), 6.59 (d, 1H, J 3.4 Hz), 7.02 (s br, 2H), 7.39 (d, 1H, J 3.4 Hz), 8.06 (s, 1H).

13C NMR (DMSO-d₆): 19.40, 59.56, 77.24, 79.29, 90.15, 91.92, 99.88, 102.39, 121.17, 149.80, 151.77, 157.47.

¹⁹F NMR (DMSO-d₆): δ14.66 (m).

ES-MS: Found: 283.1 (M+H⁺); calculated for C₁₂H₁₅FN₄O₃+H⁺: 283.1

Representative Preparation of Nucleoside Amidites

Exocyclic moieties, e.g., exocyclic amino moieties, on the heterocyclic moiety (also referenced as the base or nucleobase) of nucleosides are protected during oligonucleotide synthesis utilizing blocking groups as are know in the art, e.g., benzoyl blocking group for protection of amines. Further for those nucleoside units that include a hydroxyl group on the sugar moiety of the nucleoside, appropriate hydroxyl blocking groups, e.g., t-butylsilyl, are utilized to protect the hydroxyl group during oligonucleotide synthesis, also as is know is the art of oligonucleotide synthesis.

### Example 225

Nucleoside Phosphoramidites for Oligonucleotide Synthesis Deoxy and 2′-alkoxy amidites

2′-Deoxy and 2′-methoxy beta-cyanoethyldiisopropyl phosphoramidites were purchased from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, optimized synthesis cycles were developed that incorporate multiple steps coupling longer wait times relative to standard synthesis cycles.

The following abbreviations are used in the text: thin layer chromatography (TLC), melting point (MP), high pressure liquid chromatography (HPLC), Nuclear Magnetic Resonance (NMR), argon (Ar), methanol (MeOH), dichloromethane , (CH₂Cl₂), triethylamine (TEA), dimethyl formamide (DMF), ethyl acetate (EtOAc), dimethyl sulfoxide (DMSO), tetrahydrofuran (THF).

Oligonucleotides containing 5-methyl-2′-deoxycytidine (5-Me-dC) nucleotides were synthesized according to published methods (Sanghvi, et. al., Nucleic Acids Research, 1993, 21, 3197-3203) using commercially available phosphoramidites (Glen Research, Sterling Va. or ChemGenes, Needham Mass.) or prepared as follows:

### Example 226

5′-O-Dimethoxytrityl-thymidine intermediate for 5-methyl dC amidite

To a 50 L glass reactor equipped with air stirrer and Ar gas line was added thymidine (1.00 kg, 4.13 mol) in anhydrous pyridine (6 L) at ambient temperature. Dimethoxytrityl (DMT) chloride (1.47 kg, 4.34 mol, 1.05 eq) was added as a solid in four portions over 1 h. After 30 min, TLC indicated approx. 95% product, 2% thymidine, 5% DMT reagent and by-products and 2% 3′,5′-bis DMT product (R_{f} in EtOAc 0.45, 0.05, 0.98, 0.95 respectively). Saturated sodium bicarbonate (4 L) and CH₂Cl₂ were added with stirring (pH of the aqueous layer 7.5). An additional 18 L of water was added, the mixture was stirred, the phases were separated, and the organic layer was transferred to a second 50 L vessel. The aqueous layer was extracted with additional CH₂Cl₂ (2×2 L). The combined organic layer was washed with water (10 L) and then concentrated in a rotary evaporator to approx. 3.6 kg total weight. This was redissolved in CH₂Cl₂ (3.5 L), added to the reactor followed by water (6 L) and hexanes (13 L). The mixture was vigorously stirred and seeded to give a fine white suspended solid starting at the interface. After stirring for 1 h, the suspension was removed by suction through a ½″ diameter teflon tube into a 20 L suction flask, poured onto a 25 cm Coors Buchner funnel, washed with water (2×3 L) and a mixture of hexanes- CH₂Cl₂ (4:1, 2×3 L) and allowed to air dry overnight in pans (1″ deep). This was further dried in a vacuum oven (75° C., 0.1 mm Hg, 48 h) to a constant weight of 2072 g (93%) of a white solid, (mp 122-124° C.). TLC indicated a trace contamination of the bis DMT product. NMR spectroscopy also indicated that 1-2 mole percent pyridine and about 5 mole percent of hexanes was still present.

5′-O-Dimethoxytrityl-2′-deoxy-5-methylcytidine intermediate for 5-methyl-dC amidite

To a 50 L Schott glass-lined steel reactor equipped with an electric stirrer, reagent addition pump (connected to an addition funnel), heating/cooling system, internal thermometer and an Ar gas line was added 5′-O-dimethoxytrityl-thymidine (3.00 kg, 5.51 mol), anhydrous acetonitrile (25 L) and TEA (12.3 L, 88.4 mol, 16 eq). The mixture was chilled with stirring to −10° C. internal temperature (external −20° C). Trimethylsilylchloride (2.1 L, 16.5 mol, 3.0 eq) was added over 30 minutes while maintaining the internal temperature below −5°′ C., followed by a wash of anhydrous acetonitrile (1 L). Note: the reaction is mildly exothermic and copious hydrochloric acid fumes form over the course of the addition. The reaction was allowed to warm to 0° C. and the reaction progress was confirmed by TLC (EtOAc-hexanes 4:1; R_{f} 0.43 to 0.84 of starting material and silyl product, respectively). Upon completion, triazole (3.05 kg, 44 mol, 8.0 eq) was added the reaction was cooled to −20° C. internal temperature (external −30° C.). Phosphorous oxychloride (1035 mL, 11.1 mol, 2.01 eq) was added over 60 min so as to maintain the temperature between −20° C. and −10° C. during the strongly exothermic process, followed by a wash of anhydrous acetonitrile (1 L). The reaction was warmed to 0° C. and stirred for 1 h. TLC indicated a complete conversion to the triazole product (R_{f} 0.83 to 0.34 with the product spot glowing in long wavelength UV light). The reaction mixture was a peach-colored thick suspension, which turned darker red upon warming without apparent decomposition. The reaction was cooled to −15° C. internal temperature and water (5 L) was slowly added at a rate to maintain the temperature below +10° C. in order to quench the reaction and to form a homogenous solution. (Caution: this reaction is initially very strongly exothermic). Approximately one-half of the reaction volume (22 L) was transferred by air pump to another vessel, diluted with EtOAc (12 L) and extracted with water (2×8 L). The combined water layers were back-extracted with EtOAc (6 L). The water layer was discarded and the organic layers were concentrated in a 20 L rotary evaporator to an oily foam. The foam was coevaporated with anhydrous acetonitrile (4 L) to remove EtOAc. (note: dioxane may be used instead of anhydrous acetonitrile if dried to a hard foam). The second half of the reaction was treated in the same way. Each residue was dissolved in dioxane (3 L) and concentrated ammonium hydroxide (750 mL) was added. A homogenous solution formed in a few minutes and the reaction was allowed to stand overnight (although the reaction is complete within 1 h).

TLC indicated a complete reaction (product R_{f} 0.35 in EtOAc-MeOH 4:1). The reaction solution was concentrated on a rotary evaporator to a dense foam. Each foam was slowly redissolved in warm EtOAc (4 L; 50° C.), combined in a 50 L glass reactor vessel, and extracted with water (2×4 L) to remove, the triazole by-product. The water was back-extracted with EtOAc (2 L). The organic layers were combined and concentrated to about 8 kg total weight, cooled to 0° C. and seeded with crystalline product. After 24 hours, the first crop was collected on a 25 cm Coors Buchner funnel and washed repeatedly with EtOAc (3×3 L) until a white powder was left and then washed with ethyl ether (2×3 L). The solid was put in pans (1″ deep) and allowed to air dry overnight. The filtrate was concentrated to an oil, then redissolved in EtOAc (2 L), cooled and seeded as before. The second crop was collected and washed as before (with proportional solvents) and the filtrate was first extracted with water (2×1 L) and then concentrated to an oil. The residue was dissolved in EtOAc (1 L) and yielded a third crop which was treated as above except that more washing was required to remove a yellow oily layer.

After air-drying, the three crops were dried in a vacuum oven (50° C., 0.1 mm Hg, 24 h) to a constant weight (1750, 600 and 200 g, respectively) and combined to afford 2550 g (85%) of a white crystalline product (MP 215-217° C.) when TLC and NMR spectroscopy indicated purity. The mother liquor still contained mostly product (as determined by TLC) and a small amount of triazole (as determined by NMR spectroscopy), bis DMT product and unidentified minor impurities. If desired, the mother liquor can be purified by silica gel chromatography using a gradient of MeOH (0-25%) in EtOAc to further increase the yield.

5′-O-Dimethoxytrityl-2′-deoxy-N4-benzoyl-5-methylcytidine penultimate intermediate for 5-methyl dC amidite

Crystalline 5′-O-dimethoxytrityl-5-methyl-2′-deoxycytidine (2000 g, 3.68 mol) was dissolved in anhydrous DMF (6.0 kg) at ambient temperature in a 50 L glass reactor vessel equipped with an air stirrer and argon line. Benzoic anhydride (Chem Impex not Aldrich, 874 g, 3.86 mol, 1.05 eq) was added and the reaction was stirred at ambient temperature for 8 h. TLC (CH₂Cl₂-EtOAc; CH₂Cl₂-EtOAc 4:1; R_{f} 0.25) indicated approx. 92% complete reaction. An additional amount of benzoic anhydride (44 g, 0.19 mol) was added. After a total of 18 h, TLC indicated approx. 96% reaction completion. The solution was diluted with EtOAc (20 L), TEA (1020 mL, 7.36 mol, ca 2.0 eq) was added with stirring, and the mixture was extracted with water (15 L, then 2×10 L). The aqueous layer was removed (no back-extraction was needed) and the organic layer was concentrated in 2×20 L rotary evaporator flasks until a foam began to form. The residues were coevaporated with acetonitrile (1.5 L each) and dried (0.1 mm Hg, 25° C., 24 h) to 2520 g of a dense foam. High pressure liquid chromatography (HPLC) revealed a contamination of 6.3% of N4, 3′-O-dibenzoyl product, but very little other impurities.

The product was purified by Biotage column chromatography (5 kg Biotage) prepared with 65:35:1 hexanes-EtOAc-TEA (4L). The crude product (800 g),dissolved in CH₂Cl₂ (2 L), was applied to the column. The column was washed with the 65:35:1 solvent mixture (20 kg), then 20:80:1 solvent mixture (10 kg), then 99:1 EtOAc:TEA (17 kg). The fractions containing the product were collected, and any fractions containing the product and impurities were retained to be resubjected to column chromatography. The column was re-equilibrated with the original 65:35:1 solvent mixture (17 kg). A second batch of crude product (840 g) was applied to the column as before. The column was washed with the following solvent gradients: 65:35:1 (9 kg), 55:45:1 (20 kg), 20:80:1 (10 kg), and 99:1 EtOAc:TEA(15 kg). The column was reequilibrated as above, and a third batch of the crude product (850 g) plus impure fractions recycled from the two previous columns (28 g) was purified following the procedure for the second batch. The fractions containing pure product combined and concentrated on a 20 L rotary evaporator, co-evaporated with acetontirile (3 L) and dried (0.1 mm Hg, 48 h, 25° C.) to a constant weight of 2023 g (85%) of white foam and 20 g of slightly contaminated product from the third run. HPLC indicated a purity of 99.8% with the balance as the dibenzoyl product.

[5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite)

5′-O-(4,4′-Dimethoxytriphenylmethyl)-2′-deoxy-N⁴-benzoyl-5-methylcytidine (998 g, 1.5 mol) was dissolved in anhydrous DMF (2 L). The solution was co-evaporated with toluene (300 ml) at 50° C. under reduced pressure, then cooled to room temperature and 2-cyanoethyl tetraisopropylphosphorodiamidite (680 g, 2.26 mol) and tetrazole (52.5 g, 0.75 mol) were added. The mixture was shaken until all tetrazole was dissolved, N-methylimidazole (15 ml) was added and the mixture was left at room temperature for 5 hours. TEA (300 ml) was added, the mixture was diluted with DMF (2.5 L) and water (600 ml), and extracted with hexane (3×3 L). The mixture was diluted with water (1.2 L) and extracted with a mixture of toluene (7.5 L) and hexane (6 L). The two layers were separated, the upper layer was washed with DMF-water (7:3 v/v, 3×2 L) and water (3×2 L), and the phases were separated. The organic layer was dried (Na₂SO₄), filtered and rotary evaporated. The residue was co-evaporated with acetonitrile (2×2 L) under reduced pressure and dried to a constant weight (25° C., 0.1 mm Hg, 40 h) to afford 1250 g an off-white foam solid (96%).

### Example 227

2′-Fluoro amidites

2′-Fluorodeoxyadenosine amidites

2′-fluoro oligonucleotides were synthesized as described previously [Kawasaki, et. al., J. Med. Chem., 1993, 36, 831-841] and U.S. Pat. No. 5,670,633, herein incorporated by reference. The preparation of 2′-fluoropyrimidines containing a 5-methyl substitution are described in U.S. Pat. No. 5,861,493. Briefly, the protected nucleoside N6-benzoyl-2′-deoxy-2′-fluoroadenosine was synthesized utilizing commercially available 9-beta-D-arabinofuranosyladenine as starting material and whereby the 2′-alpha-fluoro atom is introduced by a S_{N}2-displacement of a 2′-beta-triflate group. Thus N6-benzoyl-9-beta-D-arabinofuranosyladenine was selectively protected in moderate yield as the 3′,5′-ditetrahydropyranyl (THP) intermediate. Deprotection of the THP and N6-benzoyl groups was accomplished using standard methodologies to obtain the 5′-dimethoxytrityl-(DMT) and 5′-DMT-3′-phosphoramidite intermediates.

2′-Fluorodeoxyguanosine

The synthesis of 2′-deoxy-2′-fluoroguanosine was accomplished using tetraisopropyldisiloxanyl (TPDS) protected 9-beta-D-arabinofuranosylguanine as starting material, and conversion to the intermediate isobutyryl-arabinofuranosylguanosine. Alternatively, isobutyryl-arabinofuranosylguanosine was prepared as described by Ross et al., (Nucleosides & Nucleosides, 16, 1645, 1997). Deprotection of the TPDS group was followed by protection of the hydroxyl group with THP to give isobutyryl di-THP protected arabinofuranosylguanine. Selective O-deacylation and triflation was followed by treatment of the crude product with fluoride, then deprotection of the THP groups. Standard methodologies were used to obtain the 5′-DMT- and 5′-DMT-3′-phosphoramidites.

2′-Fluorouridine

Synthesis of 2′-deoxy-2′-fluorouridine was accomplished by the modification of a literature procedure in which 2,2′-anhydro-1-beta-D-arabinofuranosyluracil was treated with 70% hydrogen fluoride-pyridine. Standard procedures were used to obtain the 5′-DMT and 5′-DMT-3′phosphoramidites.

2′-Fluorodeoxycytidine

2′-deoxy-2′-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′phosphoramidites.

### Example 228

2′-O-(2-Methoxyethyl) modified amidites

2′-O-Methoxyethyl-substituted nucleoside amidites (otherwise known as MOE amidites) are prepared as follows, or alternatively, as per the methods of Martin, P., (Helvetica Chimica Acta, 1995, 78, 486-504).

2′-O-(2-Methoxyethyl)-5-methyluridine intermediate

2,2′-Anhydro-5-methyl-uridine (2000 g, 8.32 mol), tris(2-methoxyethyl)borate (2504 g, 10.60 mol), sodium bicarbonate (60 g, 0.70 mol) and anhydrous 2-methoxyethanol (5 L) were combined in a 12 L three necked flask and heated to 130° C. (internal temp) at atmospheric pressure, under an argon atmosphere with stirring for 21 h. TLC indicated a complete reaction. The solvent was removed under reduced pressure until a sticky gum formed (50-85° C. bath temp and 100-11 mm Hg) and the residue was redissolved in water (3 L) and heated to boiling for 30 min in order the hydrolyze the borate esters. The water was removed under reduced pressure until a foam began to form and then the process was repeated. HPLC indicated about 77% product, 15% dimer (5′ of product attached to 2′ of starting material) and unknown derivatives, and the balance was a single unresolved early eluting peak.

The gum was redissolved in brine (3 L), and the flask was rinsed with additional brine (3 L). The combined aqueous solutions were extracted with chloroform (20 L) in a heavier-than continuous extractor for 70 h. The chloroform layer was concentrated by rotary evaporation in a 20 L flask to a sticky foam (2400 g). This was coevaporated with MeOH (400 mL) and EtOAc (8 L) at 75° C. and 0.65 atm until the foam dissolved at which point the vacuum was lowered to about 0.5 atm. After 2.5 L of distillate was collected a precipitate began to form and the flask was removed from the rotary evaporator and stirred until the suspension reached ambient temperature. EtOAc (2 L) was added and the slurry was filtered on a 25 cm table top Buchner funnel and the product was washed with EtOAc (3×2 L). The bright white solid was air dried in pans for 24 h then further dried in a vacuum oven (50° C., 0.1 mm Hg, 24 h) to afford 1649 g of a white crystalline solid (mp 115.5-116.5° C.).

The brine layer in the 20 L continuous extractor was further extracted for 72 h with recycled chloroform. The chloroform was concentrated to 120 g of oil and this was combined with the mother liquor from the above filtration (225 g), dissolved in brine (250 mL) and extracted once with chloroform (250 mL). The brine solution was continuously extracted and the product was crystallized as described above to afford an additional 178 g of crystalline product containing about 2% of thymine. The combined yield was 1827 g (69.4%). HPLC indicated about 99.5% purity with the balance being the dimer.

5′-O-DMT-2′-O-(2-methoxyethyl)-5-methyluridine penultimate intermediate

In a 50 L glass-lined steel reactor, 2′-O-(2-methoxyethyl)-5-methyl-uridine (MOE-T, 1500 g, 4.738 mol), lutidine (1015 g, 9.476 mol) were dissolved in anhydrous acetonitrile (15 L). The solution was stirred rapidly and chilled to −10° C. (internal temperature). Dimethoxytriphenylmethyl chloride (1765.7 g, 5.21 mol) was added as a solid in one portion. The reaction was allowed to warm to −2° C. over 1 h. (Note: The reaction was monitored closely by TLC (EtOAc) to determine when to stop the reaction so as to not generate the undesired bis-DMT substituted side product). The reaction was allowed to warm from −2 to 3° C. over 25 min. then quenched by adding MeOH (300 mL) followed after 10 min by toluene (16 L) and water (16 L). The solution was transferred to a clear 50 L vessel with a bottom outlet, vigorously stirred for 1 minute, and the layers separated. The aqueous layer was removed and the organic layer was washed successively with 10% aqueous citric acid (8 L) and water (12 L). The product was then extracted into the aqueous phase by washing the toluene solution with aqueous sodium hydroxide (0.5N, 16 L and 8 L). The combined aqueous layer was overlayed with toluene (12 L) and solid citric acid (8 moles, 1270 g) was added with vigorous stirring to lower the pH of the aqueous layer to 5.5 and extract the product into the toluene. The organic layer was washed with water (10 L) and TLC of the organic layer indicated a trace of DMT-O-Me, bis DMT and dimer DMT.

The toluene solution was applied to a silica gel column (6 L sintered glass funnel containing approx. 2 kg of silica gel slurried with toluene (2 L) and TEA(25 mL)) and the fractions were eluted with toluene (12 L) and EtOAc (3×4 L) using vacuum applied to a filter flask placed below the column. The first EtOAc fraction containing both the desired product and impurities were resubjected to column chromatography as above. The clean fractions were combined, rotary evaporated to a foam, coevaporated with acetonitrile (6 L) and dried in a vacuum oven (0.1 mm Hg, 40 h, 40° C.) to afford 2850 g of a white crisp foam. NMR spectroscopy indicated a 0.25 mole % remainder of acetonitrile (calculates to be approx. 47 g) to give a true dry weight of 2803 g (96%). HPLC indicated that the product was 99.41% pure, with the remainder being 0.06 DMT-O-Me, 0.10 unknown, 0.44 bis DMT, and no detectable dimer DMT or 3′-O-DMT.

[5′-O-(4,4′-Dimethoxyytriphenylmethyl)-2′-O-(2-methoxyethyl)-5-methyluridine-3′-O-yl]-2-cyanoethyl-N,N-diisopropylphosphoramidite (MOE T amidite)

5′-O-(4,4′-Dimethoxytriphenylmethyl)-2′-O-(2-methoxyethyl)-5-methyluridine (1237 g, 2.0 mol) was dissolved in anhydrous DMF (2.5 L). The solution was co-evaporated with toluene (200 ml) at 50° C. under reduced pressure, then cooled to room temperature and 2-cyanoethyl tetraisopropylphosphorodiamidite (900 g, 3.0 mol) and tetrazole (70 g, 1.0 mol) were added. The mixture was shaken until all tetrazole was dissolved, N-methylimidazole (20 ml) was added and the solution was left at room temperature for 5 hours. TEA (300 ml) was added, the mixture was diluted with DMF (3.5 L) and water (600 ml) and extracted with hexane (3×3 L). The mixture was diluted with water (1.6 L) and extracted with the mixture of toluene (12 L) and hexanes (9 L). The upper layer was washed with DMF-water (7:3 v/v, 3×3 L) and water (3×3 L). The organic layer was dried (Na₂SO₄), filtered and evaporated. The residue was co-evaporated with acetonitrile (2×2 L) under reduced pressure and dried in a vacuum oven (25° C., 0.1 mm Hg, 40 h) to afford 1526 g of an off-white foamy solid (95%).

5′-O-Dimethoxytrityl-2′-O-(2-methoxyethyl)-5-methylcytidine intermediate

To a 50 L Schott glass-lined steel reactor equipped with an electric stirrer, reagent addition pump (connected to an addition funnel), heating/cooling system, internal thermometer and argon gas line was added 5′-O-dimethoxytrityl-2′-O-(2-methoxyethyl)-5-methyl-uridine (2.616 kg, 4.23 mol, purified by base extraction only and no scrub column), anhydrous acetonitrile (20 L), and TEA (9.5 L, 67.7 mol, 16 eq). The mixture was chilled with stirring to −10° C. internal temperature (external −20° C.). Trimethylsilylchloride (1.60 L, 12.7 mol, 3.0 eq) was added over 30 min. while maintaining the internal temperature below −5° C., followed by a wash of anhydrous acetonitrile (1 L). (Note: the reaction is mildly exothermic and copious hydrochloric acid fumes form over the course of the addition). The reaction was allowed to warm to 0° C. and the reaction progress was confirmed by TLC (EtOAc, R_{f} 0.68 and 0.87 for starting material and silyl product, respectively). Upon completion, triazole (2.34 kg, 33.8 mol, 8.0 eq) was added the reaction was cooled to −20° C. internal temperature (external −30° C.). Phosphorous oxychloride (793 mL, 8.51 mol, 2.01 eq) was added slowly over 60 min so as to maintain the temperature between −20° C. and −10° C. (note: strongly exothermic), followed by a wash of anhydrous acetonitrile (1 L). The reaction was warmed to 0° C. and stirred for 1 h, at which point it was an off-white thick suspension. TLC indicated a complete conversion to the triazole product (EtOAc, R_{f} 0.87 to 0.75 with the product spot glowing in long wavelength UV light). The reaction was cooled to −15° C. and water (5 L) was slowly added at a rate to maintain the temperature below +10° C. in order to quench the reaction and to form a homogenous solution. (Caution: this reaction is initially very strongly exothermic). Approximately one-half of the reaction volume (22 L) was transferred by air pump to another vessel, diluted with EtOAc (12 L) and extracted with water (2×8 L). The second half of the reaction was treated in the same way. The combined aqueous layers were back-extracted with EtOAc (8 L) The organic layers were combined and concentrated in a 20 L rotary evaporator to an oily foam. The foam was coevaporated with anhydrous acetonitrile (4 L) to remove EtOAc. (note: dioxane may be used instead of anhydrous acetonitrile if dried to a hard foam). The residue was dissolved in dioxane (2 L) and concentrated ammonium hydroxide (750 mL) was added. A homogenous solution formed in a few minutes and the reaction was allowed to stand overnight

TLC indicated a complete reaction (CH₂Cl₂-acetone-MeOH, 20:5:3, R_{f} 0.51). The reaction solution was concentrated on a rotary evaporator to a dense foam and slowly redissolved in warm CH₂Cl₂ (4 L, 40° C.) and transferred to a 20 L glass extraction vessel equipped with a air-powered stirrer. The organic layer was extracted with water (2×6 L) to remove the triazole by-product. (Note: In the first extraction an emulsion formed which took about 2 h to resolve). The water layer was back-extracted with CH₂Cl₂ (2×2 L), which in turn was washed with water (3 L). The combined organic layer was concentrated in 2×20 L flasks to a gum and then recrystallized from EtOAc seeded with crystalline product. After sitting overnight, the first crop was collected on a 25 cm Coors Buchner funnel and washed repeatedly with EtOAc until a white free-flowing powder was left (about 3×3 L). The filtrate was concentrated to an oil recrystallized from EtOAc, and collected as above. The solid was air-dried in pans for 48 h, then further dried in a vacuum oven (50° C., 0.1 mm Hg, 17 h) to afford 2248 g of a bright white, dense solid (86%). An HPLC analysis indicated both crops to be 99.4% pure and NMR spectroscopy indicated only a faint trace of EtOAc remained.

5′-O-dimethoxytrityl-2′-O-(2-methoxyethyl)-N4-benzoyl-5-methyl-cytidine penultimate intermediate:

Crystalline 5′-O-dimethoxytrityl-2′-O-(2-methoxyethyl)-5-methyl-cytidine (1000 g, 1.62 mol) was suspended in anhydrous DMF (3 kg) at ambient temperature and stirred under an Ar atmosphere. Benzoic anhydride (439.3 g, 1.94 mol) was added in one portion. The solution clarified after 5 hours and was stirred for 16 h. HPLC indicated 0.45% starting material remained (as well as 0.32% N4, 3′-O-bis Benzoyl). An additional amount of benzoic anhydride (6.0 g, 0.0265 mol) was added and after 17 h, HPLC indicated no starting material was present. TEA (450 mL, 3.24 mol) and toluene (6 L) were added with stirring for 1 minute. The solution was washed with water (4×4 L), and brine (2×4 L). The organic layer was partially evaporated on a 20 L rotary evaporator to remove 4 L of toluene and traces of water. HPLC indicated that the bis benzoyl side product was present as a 6% impurity. The residue was diluted with toluene (7 L) and anhydrous DMSO (200 mL, 2.82 mol) and sodium hydride (60% in oil, 70 g, 1.75 mol) was added in one portion with stirring at ambient temperature over 1 h. The reaction was quenched by slowly adding then washing with aqueous citric acid (10%, 100 mL over 10 min, then 2×4 L), followed by aqueous sodium bicarbonate (2%, 2 L), water (2×4 L) and brine (4 L). The organic layer was concentrated on a 20 L rotary evaporator to about 2 L total volume. The residue was purified by silica gel column chromatography (6 L Buchner funnel containing 1.5 kg of silica gel wetted with a solution of EtOAc-hexanes-TEA(70:29:1)). The product was eluted with the same solvent (30 L) followed by straight EtOAc (6 L). The fractions containing the product were combined, concentrated on a rotary evaporator to a foam and then dried in a vacuum oven (50° C., 0.2 mm Hg, 8 h) to afford 1155 g of a crisp, white foam (98%). HPLC indicated a purity of >99.7%.

[5-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite)

5′-O-(4,4′-Dimethoxytriphenylmethyl)-2′-O-(2-methoxyethyl)-N⁴-benzoyl-5-methylcytidine (1082 g, 1.5 mol) was dissolved in anhydrous DMF (2 L) and co-evaporated with toluene (300 ml) at 50° C. under reduced pressure. The mixture was cooled to room temperature and 2-cyanoethyl tetraisopropylphosphorodiamidite (680 g, 2.26 mol) and tetrazole (52.5 g, 0.75 mol) were added. The mixture was shaken until all tetrazole was dissolved, N-methylimidazole (30 ml) was added, and the mixture was left at room temperature for 5 hours. TEA (300 ml) was added, the mixture was diluted with DMF (1 L) and water (400 ml) and extracted with hexane (3×3 L). The mixture was diluted with water (1.2 L) and extracted with a mixture of toluene (9 L) and hexanes (6 L). The two layers were separated and the upper layer was washed with DMF-water (60:40 v/v, 3×3 L) and water (3×2 L). The organic layer was dried (Na₂SO₄), filtered and evaporated. The residue was co-evaporated with acetonitrile (2×2 L) under reduced pressure and dried in a vacuum oven (25° C., 0.1 mm Hg, 40 h) to afford 1336 g of an/off-white foam (97%).

[5′-O-(4,4′-Dimethoxetriphenylmethyl)-2′-O-(2-methoxyethyl)-N6-benzoyladenosin-3′-O-yl]-2-cyanoethyl-N,N-diisopropylphosphoramidite (MOE A amdite)

5′-O-(4,4′-Dimethoxytriphenylmethyl)-2′-O-(2-methoxyethyl)-N⁶-benzoyladenosine (purchased from Reliable Biopharmaceutical, St. Lois, Mo.), 1098 g, 1.5 mol) was dissolved in anhydrous DMF (3 L) and co-evaporated with toluene (300 ml) at 50° C. The mixture was cooled to room temperature and 2-cyanoethyl tetraisopropylphosphorodiamidite (680 g, 2.26 mol) and tetrazole (78.8 g, 1.24 mol) were added. The mixture was shaken until all tetrazole was dissolved, N-methylimidazole (30 ml) was added, and mixture was left at room temperature for 5 hours. TEA (300 ml) was added, the mixture was diluted with DMF (1 L) and water (400 ml) and extracted with hexanes (3×3 L). The mixture was diluted with water (1.4 L) and extracted with the mixture of toluene (9 L) and hexanes (6 L). The two layers were separated and the upper layer was washed with DMF-water (60:40, v/v, 3×3 L) and water (3×2 L). The organic layer was dried (Na₂SO₄), filtered and evaporated to a sticky foam. The residue was co-evaporated with acetonitrile (2.5 L) under reduced pressure and dried in a vacuum oven (25° C., 0.1 mm Hg, 40 h) to afford 1350 g of an off-white foam solid (96%).

[5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite (MOE G amidite)

5′-O-(4,4′-Dimethoxytriphenylmethyl)-2′-O-(2-methoxyethyl)-N⁴-isobutyrlguanosine (purchased from Reliable Biopharmaceutical, St. Louis, Mo., 1426 g, 2.0 mol) was dissolved in anhydrous DMF (2 L). The solution was co-evaporated with toluene (200 ml) at 50° C., cooled to room temperature and 2-cyanoethyl tetraisopropylphosphorodiamidite (900 g, 3.0 mol) and tetrazole (68 g, 0.97 mol) were added. The mixture was shaken until all tetrazole was dissolved, N-methylimidazole (30 ml) was added, and the mixture was left at room temperature for 5 hours. TEA (300 ml) was added, the mixture was diluted with DMF (2 L) and water (600 ml) and extracted with hexanes (3×3 L). The mixture was diluted with water (2 L) and extracted with a mixture of toluene (10 L) and hexanes (5 L). The two layers were separated and the upper layer was washed with DMF-water (60:40, v/v, 3×3 L). EtOAc (4 L) was added and the solution was washed with water (3×4 L). The organic layer was dried (Na₂SO₄), filtered and evaporated to approx. 4 kg. Hexane (4 L) was added, the mixture was shaken for 10 min, and the supernatant liquid was decanted. The residue was co-evaporated with acetonitrile (2×2 L) under reduced pressure and dried in a vacuum oven (25° C., 0.1 mm Hg, 40 h) to afford 1660 g of an off-white foamy solid (91%).

### Example 229

2′-O-(Dimethylaminooxyethyl) nucleoside amidites

2′-(Dimethylaminooxyethoxy) nucleoside amidites

2′-(Dimethylaminooxyethoxy) nucleoside amidites (also known in the art as 2′-O-(dimethylaminooxyethyl) nucleoside amidites) are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

5′-O-tert-Butyldiphenylsilyl-O²-2′-anhydro-5-methyluridine

O²-2′-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 ml) at ambient temperature under an argon atmosphere and with mechanical stirring. teyl-Butyldiphenylchlorosilane (125.8 g, 119.0 mL, 1.1 eq, 0.458 mmol) was added in one portion. The reaction was stirred for 16 h at ambient temperature. TLC (R_{f} 0.22, EtOAc) indicated a complete reaction. The solution was concentrated under reduced pressure to a thick oil. This was partitioned between CH₂Cl₂ (1 L) and saturated sodium bicarbonate (2×1 L) and brine (1 L). The organic layer was dried over sodium sulfate, filtered, and concentrated under reduced pressure to a thick oil. The oil was dissolved in a 1:1 mixture of EtOAc and ethyl ether (600 mL) and cooling the solution to −10° C. afforded a white crystalline solid which was collected by filtration, washed with ethyl ether (3×200 mL) and dried (40° C., 1 mm Hg, 24 h) to afford 149 g of white solid (74.8%). TLC and NMR spectroscopy were consistent with pure product.

5′-O-tert-Butyldiphenylsilyl-2′-O-(2-hydroxyethyl)-5-methyluridine

In the fume hood, ethylene glycol (350 mL, excess) was added cautiously with manual stirring to a 2 L stainless steel pressure reactor containing borane in tetrahydrofuran (1.0 M, 2.0 eq, 622 mL). (Caution : evolves hydrogen gas). 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure <100 psig). The reaction vessel was cooled to ambient temperature and opened. TLC (EtOAc, R_{f} 0.67 for desired product and R_{f} 0.82 for ara-T side product) indicated about 70% conversion to the product. The solution was concentrated under reduced pressure (10 to 1 mm Hg) in a warm water bath (40-100° C.) with the more extreme conditions used to remove the ethylene glycol. (Alternatively, once the THF has evaporated the solution can be diluted with water and the product extracted into EtOAc). The residue was purified by column chromatography (2 kg silica gel, EtOAc-hexanes gradient 1:1 to 4:1). The appropriate fractions were combined, evaporated and dried to afford 84 g of a white crisp foam (50%), contaminated starting material (14.4 g, 12% recovery) and pure reusable starting material (20 g, 13% recovery). TLC and NMR spectroscopy were consistent with 99% pure product.

2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol) and dried over P₂O₅ under high vacuum for two days at 40° C. The reaction mixture was flushed with argon and dissolved in dry THF (369.8 mL, Aldrich, sure seal bottle). Diethyl-azodicarboxylate (6.98 mL, 44.36 mmol) was added dropwise to the reaction mixture with the rate of addition maintained such that the resulting deep red coloration is just discharged before adding the next drop. The reaction mixture was stirred for 4 hrs., after which time TLC (EtOAc:hexane, 60:40) indicated that the reaction was complete. The solvent was evaporated in vacuuo and the residue purified by flash column chromatography (eluted with 60:40 EtOAc:hexane), to yield 2′-O-([2-phthalimidoxy)ethyl]-5′-t-butyldiphenylsilyl-5-methyluridine as white foam (21.819 g, 86%) upon rotary evaporation.

5,′-O-tert-butyldiphenylsilyl-20-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate washed with ice cold CH₂Cl₂, and the combined organic phase was washed with water and brine and dried (anhydrous Na₂SO₄). The solution was filtered and evaporated to afford 2′-O-(aminooxyethyl) thymidine, which was then dissolved in MeOH (67.5 mL). Formaldehyde (20% aqueous solution, w/w, 1.1 eq.) was added and the resulting mixture was stirred for 1 h. The solvent was removed under vacuum and the residue was purified by column chromatography to yield 5′-O-tert-butyldiphenylsilyl-2′-O-[(2-formadoximinooxy) ethyl]-5-methyluridine as white foam (1.95 g, 78%) upon rotary evaporation.

5′-O-tert-Butyldiphenylsilyl-2′-O-[N,N dimethylaminooxyethyl]-5-methyluridine

5′-O-tert-butyldiphenylsilyl-2′-O-[(2-formadoximinooxy)ethyl]-5-methyluridine (1.77 g, 3.12 mmol) was dissolved in a solution of 1 M pyridinium p-toluenesulfonate (PPTS) in dry MeOH (30.6 mL) and cooled to 10° C. under inert atmosphere. Sodium cyanoborohydride (0.39 g, 6.13 mmol) was added and the reaction mixture was stirred. After 10 minutes the reaction was warmed to room temperature and stirred for 2 h. while the progress of the reaction was monitored by TLC (5% MeOH in CH₂Cl₂). Aqueous NaHCO₃ solution (5%, 10 mL) was added and the product was extracted with EtOAc (2×20 mL). The organic phase was dried over anhydrous Na₂SO₄, filtered, and evaporated to dryness. This entire procedure was repeated with the resulting residue, with the exception that formaldehyde (20% w/w, 30 mL, 3.37 mol) was added upon dissolution of the residue in the PPTS/MeOH solution. After the extraction and evaporation, the residue was purified by flash column chromatography and (eluted with 5% MeOH in CH₂Cl₂) to afford 5′-O-tert-butyldiphenylsilyl-2′-O-[N,N-dimethylaminooxyethyl]-5-methyluridine as a white foam (14.6 g, 80%) upon rotary evaporation.

2′-O-(dimethylaminooxyethyl)-5-methyluridine

Triethylamine trihydrofluoride (3.91 mL, 24.0 mmol) was dissolved in dry THF and TEA (1.67 mL, 12 mmol, dry, stored over KOH) and added to 5′-O-tert-butyldiphenylsilyl-2′-[N,N-dimethylaminooxyethyl]-5-methyluridine (1.40 g, 2.4 mmol). The reaction was stirred at room temperature for 24 hrs and monitored by TLC (5% MeOH in CH₂Cl₂). The solvent was removed under vacuum and the residue purified by flash column chromatography (eluted with 10% MeOH in CH₂Cl₂) to afford 2′-O-(dimethylaminooxyethyl)-5-methyluridine (766 mg, 92.5%) upon rotary evaporation of the solvent.

5′-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C., co-evaporated with anhydrous pyridine (20 mL), and dissolved in pyridine (11 mL) under argon atmosphere. 4-dimethylaminopyridine (26.5 mg, 2.60 mmol) and 4,4′-dimethoxytrityl chloride (880 mg, 2.60 mmol) were added to the pyridine solution and the reaction mixture was stirred at room temperature until all of the starting material had reacted. Pyridine was removed under vacuum and the residue was purified by column chromatography (eluted with 10% MeOH in CH₂Cl₂ containing a few drops of pyridine) to yield 5′-O-DMT-2′-O-(dimethylamino-oxyethyl)-5-methyluridine (1.13 g, 80%) upon rotary evaporation.

5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL), N,N-diisopropylamine tetrazonide (0.29 g, 1.67 mmol) was added and the mixture was dried over P₂O₅ under high vacuum overnight at 40° C. This was dissolved in anhydrous acetonitrile (8.4 mL) and 2-cyanoethyl-N,N,N¹,N¹-tetraisopropylphosphoramidite (2.12 mL, 6.08 mmol) was added. The reaction mixture was stirred at ambient temperature for 4 h under inert atmosphere. The progress of the reaction was monitored by TLC (hexane:EtOAc 1:1). The solvent was evaporated, then the residue was dissolved in EtOAc (70 mL) and washed with 5% aqueous NaHCO₃ (40 mL). The EtOAc layer was dried over anhydrous Na₂SO₄, filtered, and concentrated. The residue obtained was purified by column chromatography (EtOAc as eluent) to afford 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%) upon rotary evaporation.

### Example 230

2′-O-(Aminooxyethyl) nucleoside amidites

2′-(Aminooxyethoxy) nucleoside amidites (also known in the art as 2′-O-(aminooxyethyl) nucleoside amidites) are prepared as described in the following paragraphs. Adenosine, cytidine and thymidine nucleoside amidites are prepared similarly.

N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine ribosome. Multi gram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-hydroxyethyl)-5′-O-(4,4′-dimethoxytrityl)guanosine. As before the hydroxyl group may be displaced by N-hydroxyphthalimide via a Mitsunobu reaction, and the protected nucleoside may be phosphitylated as usual to yield 2-N-isobutyryl-6-O-diphenylcarbamoyl-2′-O-([2-phthalimidoxy]ethyl)-5′-O-(4,4′-dimethoxytrityl)guanosine-3′-[(2-cyanoethyl)-N,N-diisopropylphosphoramidite].

### Example 231

2′-dimethylaminoethoxyethoxy (2′-DMAEOE) nucleoside amidites

2′-dimethylaminoethoxyethoxy nucleoside amidites (also known in the art as 2′-O-dimethylaminoethoxyethyl, i.e., 2′-O—CH₂—O—CH₂—N(CH₂)₂, or 2′-DMAEOE nucleoside amidites) are prepared as follows. Other nucleoside amidites are prepared similarly.

2′-O-[2(2-N,N-dimethylaminoethoxy)ethyl]-5-methyl uridine

2[2-(Dimethylamino)ethoxylethanol (Aldrich, 6.66 g, 50 mmol) was slowly added to a solution of borane in tetrahydrofuran (1 M, 10 mL, 10 mmol) with stirring in a 100 mL bomb. (Caution: Hydrogen gas evolves as the solid dissolves). O²—,2′-anhydro-5-methyluridine (1.2 g, 5 mmol), and sodium bicarbonate (2.5 mg) were added and the bomb was sealed, placed in an oil bath and heated to 155° C. for 26 h. then cooled to room temperature. The crude solution was concentrated, the residue was diluted with water (200 mL) and extracted with hexanes (200 mL). The product was extracted from the aqueous layer with EtOAc (3×200 mL) and the combined organic layers were washed once with water, dried over anhydrous sodium sulfate, filtered and concentrated. The residue was purified by silica gel column chromatography (eluted with 5:100:2 MeOH/CH₂Cl₂/TEA) as the eluent. The appropriate fractions were combined and evaporated to afford the product as a white solid.

5′-O-dimethoxytrityl-2′-O-[2(2-N,N-dimethylaminoethoxy)ethyl)]-5-methyl uridine

To 0.5 g (1.3 mmol) of 2′-O-[2(2-N,N-dimethylaminoethoxy)ethyl)]-5-methyl uridine in anhydrous pyridine (8 mL), was added TEA (0.36 mL) and dimethoxytrityl chloride (DMT-Cl, 0.87 g, 2 eq.) and the reaction was stirred for 1 h. The reaction mixture was poured into water (200 mL) and extracted with CH₂Cl₂ (2×200 mL). The combined CH₂Cl₂ layers were washed with saturated NaHCO₃ solution, followed by saturated NaCl solution, dried over anhydrous sodium sulfate, filtered and evaporated. The residue was purified by silica gel column chromatography (eluted with 5:100:1 MeOH/CH₂Cl₂/TEA) to afford the product.

5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) were added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture was stirred overnight and the solvent evaporated. The resulting residue was purified by silica gel column chromatography with EtOAc as the eluent to afford the title compound.

### Example 232

In a like manner to Examples 222 to 228, protected nucleoside amidites of the nucleoside of Examples 1 to 143 are prepared.

Oligonucleotide Synthesis

Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄oAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. Nos. 5,256,775 or 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 233

Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 234

Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

[2′-O-Me]-[2′-deoxy]-2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

[2′-O-(2-Methoxyethyl)]-[2′-deoxy]-[2′-O-(Methoxyethyl)l Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

[2′-O-(2-Methoxyethyl)Phosphodiester]-[2′-deoxy Phosphorothioate]-[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester intemucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 235

Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32 +/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 236

Oligonucleotide Synthesis —96 Well Plate Format

Oligonucleotides were synthesized via solid phase P(III) phosphoramidite chemistry on an automated synthesizer capable of assembling 96 sequences simultaneously in a 96-well format. Phosphodiester intemucleotide linkages were afforded by oxidation with aqueous iodine. Phosphorothioate intemucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 237

Oligonucleotide Analysis —96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 238

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

Treatment With Antisense Compounds

When cells reached 70% confluency, they were treated with oligonucleotide. For cells grown in 96-well plates, wells were washed once with 100 μL OPTI-MEM™−1 reduced-serum medium (Invitrogen Corporation, Carlsbad, Calif.) and then treated with 130 μL of OPTI-MEM™−1 containing 3.75 μg/mL LIPOFECTIN™ (Invitrogen Corporation, Carlsbad, Calif.) and the desired concentration of oligonucleotide. After 4-7 hours of treatment, the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is ISIS 13920, TCCGTCATCGCTCCTCAGGG, SEQ ID NO: 1, a 2′-O-methoxyethyl gapmer (2′-O-methoxyethyls shown in bold) with a phosphorothioate backbone which is targeted to human H-ras. For mouse or rat cells the positive control oligonucleotide is ISIS 15770, ATGCATTCTGCCCCCAAGGA, SEQ ID NO: 2, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-Ha-ras (for ISIS 13920) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments.

### Example 239

Analysis of Oligonucleotide Inhibition of Expression

Antisense modulation of gene expression can be assayed in a variety of ways known in the art. For example, gene mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+mRNA. The preferred method of RNA analysis of the present invention is the use of total cellular RNA as described in other examples herein. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Protein levels can be quantitated in a variety of ways well known in the art, such as immunoprecipitation, Western blot analysis (immunoblotting), ELISA or fluorescence-activated cell sorting (FACS). Antibodies directed to a particular gene can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F.M. et al., (Current Protocols in Molecular Biology, Volume 2, pp. 11.12.1-11.12.9, John Wiley & Sons, Inc., 1997). Preparation of monoclonal antibodies is taught in, for example, Ausubel, F.M. et al., (Current Protocols in Molecular Biology, Volume 2, pp. 11.4.1-11.11.5, John Wiley & Sons, Inc., 1997).

Immunoprecipitation methods are standard in the art and can be found at, for example, Ausubel, F. M. et al., (Current Protocols in Molecular Biology, Volume 2, pp. 10.16.1-10.16.11, John Wiley & Sons, Inc., 1998). Western blot (immunoblot) analysis is standard in the art and can be found at, for example, Ausubel, F. M. et al., (Current Protocols in Molecular Biology, Volume 2, pp. 10.8.1-10.8.21, John Wiley & Sons, Inc., 1997). Enzyme-linked immunosorbent assays (ELISA) are standard in the art and can be found at, for example, Ausubel, F. M. et al., (Current Protocols in Molecular Biology, Volume 2, pp. 11.2.1-11.2.22, John Wiley & Sons, Inc., 1991).

### Example 240

Poly(A)+mRNA Isolation

Poly(A)+mRNA was isolated according to Miura et aL, (Clin. Chem., 1996, 42, 1758-1764). Other methods for poly(A)+mRNA isolation are taught in, for example, Ausubel, F. M. et al., (Current Protocols in Molecular Biology, Volume 1, pp. 4.5.1-4.5.3, John Wiley & Sons, Inc., 1993). Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Example 241

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 170 μL water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 242

Real-time Quantitative PCR Analysis of mRNA Levels

Quantitation of mRNA levels was determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR)-products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, MRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5× PCR buffer (−MgCl2), 6.6 mM MgCl2, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution. The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreenTM (Molecular Probes, Inc. Eugene, Oreg.). GAPDH expression is quantified by real time RT-PCR, by being run simultaneously with the target, multiplexing, or separately. Total RNA is quantified using RiboGreenTM RNA quantification reagent from Molecular Probes. Methods of RNA quantification by RiboGreenTM are taught in Jones, L. J., et al, (Analytical Biochemistry, 1998, 265, 368-374).

In this assay, 170 μL of RiboGreenTM working reagent (RiboGreenTM reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 480 nm and emission at 520 nm.

Probes and primers to particular gene of interest are are designed to hybridize to the gene sequence, using published sequence information, as for instance via their GenBank accession number. Forward and reverse primes and probes are selected for the gene of interest. The PCR probe is selected having a FAM -TAMRA quencher-dye pair where FAM is the fluorescent dye and TAMRA is the quencher dye. Other PCR probe can be selected as 5′ JOE—TAMRA 3′ modified probes where JOE is the fluorescent reporter dye and TAMRA is the quencher dye.

### Example 243

Northern Blot Analysis of mRNA Levels

Eighteen hours after antisense treatment, cell monolayers are washed twice with cold PBS and lysed in 1 mL RNAZOL™ (TEL-TEST “B” Inc., Friendswood, Tex.). Total RNA is prepared following manufacturer's recommended protocols. Twenty micrograms of total RNA is fractionated by electrophoresis through 1.2% agarose gels containing 1.1% formaldehyde using a MOPS buffer system (AMRESCO, Inc. Solon, Ohio). RNA is transferred from the gel to HYBOND™ −N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer is confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect the gene of interest a specific probe is prepared by PCR using the forward primer and the reverse primer. To normalize for variations in loading and transfer efficiency membranes are stripped and probed for glyceraldehyde-3-phosphate dehydrogenase (GAPDH) RNA (Clontech, Palo Alto, Calif.).

Hybridized membranes are visualized and quantitated using a PHOSPHORIMAGER™ and IMAGEQUANT™ Software V3.3 (Molecular Dynamics, Sunnyvale, Calif.). Data is normalized to GAPDH levels in untreated controls.

### Example 244

Antisense Inhibition of Genes

A series of oligonucleotides are designed to target different regions of the RNA, using published gene sequences. The selected oligonucleotides are analyzed for their effect on the mRNA levels by quantitative real-time PCR as described in the examples herein. Data are averages from two experiments. Target sites to which the preferred sequences are complementary are referred to as “preferred target regions” and are therefore preferred sites for targeting by compounds of the present invention. As these “preferred target regions” are found by experimentation to be open to, and accessible for, hybridization with the antisense compounds, one of skill in the art will recognize or be able to ascertain, using no more than routine experimentation, further embodiments of the invention that encompass other compounds that specifically hybridize to these sites and consequently inhibit the expression of the gene of interest.

### Example 245

Western Blot Analysis of Protein Levels

Western blot analysis (immunoblot analysis) is carried out using standard methods. Cells are harvested 16-20 h after oligonucleotide treatment, washed once with PBS, suspended in Laemmli buffer (100 μl/well), boiled for 5 minutes and loaded on a 16% SDS-PAGE gel. Gels are run for 1.5 hours at 150 V, and transferred to membrane for western blotting. Appropriate primary antibody directed to the gene of interest is used, with a radiolabeled or fluorescently labeled secondary antibody directed against the primary antibody species. Bands are visualized using a PHOSPHORIMAGER™ (Molecular Dynamics, Sunnyvale Calif.).

### Example 246

Nuclease Stability Determination

Nuclease stability of oligonucleotide is determined, at a concentration of 2 μM, by incubated with snake venom phosphodiesterase (0.005 IU/mL) in 50 mM Tris-HCl, pH 7.5, 8 mM MgCl₂ at 37° C. The total volume is 100 μL. At each time point 10 μL aliquots of each reaction mixture are placed in a 500 μL microfuge tubes and put in a boiling water bath for two minutes. The samples are then cooled on ice, quick spun to bring the entire volume to the bottom of the tube, and desalted on a Millipore 0.025 micron filter disk (Bedford, Mass.) that is floating in water in a 60 mm petrie dish. After 30-60 minutes on the membrane the sample is diluted with 200 μL distilled H₂O and analyzed by gel-filled capillary electrophoresis. The oligonucleotide and metabolites are separated and analyzed using the Beckman P/ACE MDQ capillary electrophoresis instrument using a 100 μm ID 30 cm coated capillary (Beckman No. 477477) with eCAP ssDNA 100-R gel (Beckman No. 477621) and Tris-Borate Urea buffer (Beckman No. 338481). The samples are injected electrokinetically using a field strength of between 5-10 kV for a duration of between 5 and 10 seconds. Separation is effected at 40° C. with an applied voltage of 15 kV. The percentage of full length oligonucleotide is calculated by integration using Caesar v. 6 software (Senetec Software, New Jersey) followed by correction for differences in extinction coefficient for oligonucleotides of different length.

### Example 247

Binding of Oligonucleotide to Human Serum Albumin

The binding of oligonucleotides of the invention to serum proteins is representative of binding of the oligonucleotides to proteins. This is determined by labeling 5′-end of each oligonucleotide with ³²P using T4 polynucleotide kinase and standard procedures. Unincorporated label is removed using a G25 column and is confirmed by polyacrylamide gel electrophoresis. A fixed concentration of labeled oligonucleotide (50 nM) is incubated with increasing concentrations of human serum albumin (Fraction V, essentially Fatty Acid Free, essentially globulin free, Sigma) and incubated at 25° C. for one hour in PBS plus 0.1 mM EDTA and 0.005% Tween 80. Experiments with longer incubation times demonstrate that full equilibrium ere achieved in less than one hour. Albumin-oligo mixtures are placed on the membranes (Ultrafree-MC 30 000, Millipore) and spun very gently at 3000 rpm (725×g) for 3-6 min until ˜20% of the volume is passed through the filter. Aliquots of the initial mix (before filtration) and the filtrate are counted in the scintillation counter. After appropriate correction for background, concentration of free and bound oligonucleotide is calculated. A low concentration of oligonucleotide, relative to albumin, allows for detection of binding to only the tightest binding site on the albumin. Thus, fraction of oligonucleotide bound cn be plotted vs. total albumin concentration and data fit to a two state model:

K_{A}+A [symbol] (OA)

where O is unbound oligonucleotide, A is unbound albumin, (OA) is the oligonucleotide-albumin complex and K_{A} is the equilibrium association constant.

### Example 248

Crystallization and Structure Determination

Crystal structures of oligonucleotides of the invention can also be determined. Optimal crystallization conditions for a modified oligonucleotide is screened by the sparse matrix crystallization technique, using the Hampton Research (Laguna Niguel, Calif.) nucleic acid mini screen. Crystals for data collection are grown by the hanging drop vapor diffusion method. Equal volumes of a 2 mM oligonucleotide solution in water and a buffer solution, containing 40 mM sodium cacodylate (pH 7.0), 80 mM potassium chloride, 12 mM spermine tetrahydrochloride and 10% (v/v) 2-methyl-2,4-pentanediol (MPD), are mixed and equilibrated against 1 mL 35% (v/v) MPD. Diffraction data to a maximum resolution of 1.2 Å is collected on a single flash-frozen (100 K) crystal at a wavelength of 1 Å on the 5-ID beamline at the Advanced Photon Source (DuPont-Northwestern-Dow Collaborative Access Team, Argonne, Ill.), using a MARCCD detector. Data is integrated and merged in the DENZO/SCALEPACK suite. The structure is solved by the molecular replacement method using the program AMORE. Crystallographic refinements are performed with the programs CNS and SHELX-97.

### Example 249

Design and Screening of Duplexed Oligomeric Compounds Targeting a Target

In accordance with the present invention, a series of nucleic acid duplexes comprising the antisense oligomeric compounds of the present invention and their complements can be designed to target a target. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| 5′ c g a g a g g c g g a c g g g a c c g T T 3′ | Antisense Strand |
| \| \| \| \| \| \| \| \| \| \| \| \| \| \| \| \| \| \| \| | |
| 3′ T T g c t c t c c g c c t g c c c t g g c 5′ | Complement Strand |

RNA strands of the duplex can be synthesized by methods disclosed herein or purchased from Dharmacon Research Inc., (Lafayette, Conn.). Once synthesized, the complementary strands are annealed. The single strands are aliquoted and diluted to a concentration of 50 μM. Once diluted, 30 μL of each strand is combined with 15 μL of a 5× solution of annealing buffer. The final concentration of said buffer is 100 mM potassium acetate, 30 mM HEPES-KOH pH 7.4, and 2mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM. This solution can be stored frozen (−20° C.) and freeze-thawed up to 5 times.

Once prepared, the duplexed antisense oligomeric compounds are evaluated for their ability to modulate a target expression. When cells reached 80% confluency, they are treated with duplexed antisense oligomeric compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense oligomeric compound at a final concentration of 200 nM. After 5 hours of treatment, the medium is replaced with fresh medium. Cells are harvested 16 hours after treatment, at which time RNA is isolated and target reduction measured by RT-PCR.

What is claimed is:

1. A compound comprising a plurality of linked nucleoside units, at least one of said nucleoside units comprising a modified nucleoside of structural formula I of the indicated stereochemical configuration: 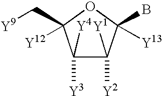
wherein B is selected from the group consisting of 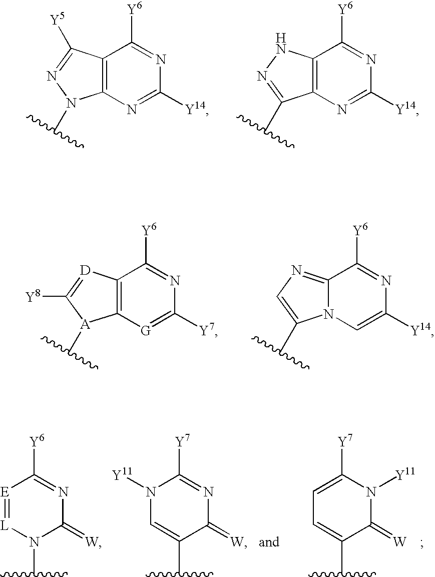
A is CH, and G is N or CH, and D is N, CH, C—CN, C—NO₂, C—C₁₋₃ alkyl, C—NHCONH₂, C—CONY¹¹Y¹¹, C—CSNY¹¹Y¹¹, C—COOY¹¹, C-hydroxy, C—C₁₋₃ alkoxy, C-amino, C—C₁₋₄ alkylamino, C-di(C₁₋₄ alkyl)amino, C-halogen, C-(1,3-oxazol-2-yl), C-(1,3-thiazol-2-yl), or C-(imidazol-2-yl); wherein alkyl is unsubstituted or substituted with one to three groups independently selected from halogen, amino, hydroxy, carboxy, or C₁₋₃ alkoxy; or
A is N, and G is CH, and D is CH, C—CN, C—NO₂, C—C₁₋₃ alkyl, C—NHCONH₂, C—CONY¹¹Y¹¹, C—CSNY¹¹Y¹¹Y¹¹, C—COOY¹¹, C-hydroxy, C—C₁₋₃ alkoxy, C-amino, C—C₁₋₄ alkylamino, C-di(C₁₋₄ alkyl)amino, C-halogen, C-(1,3-oxazol-2-yl), C-(1,3-thiazol-2-yl), or C-(imidazol-2-yl); wherein alkyl is unsubstituted or substituted with one to three groups independently selected from halogen, amino, hydroxy, carboxy, or C₁₋₃ alkoxy;
E is N and L is CY⁵; or E is CY⁵ and L is N;
W is O or S;
Y¹, Y², Y³ and Y⁴ each independently are a linkage to a further of said nucleoside units of said compound; hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y⁵ is H, C₁₋₆ alkyl, C₂₋₆ alkenyl, C₂₋₆ alkynyl, C₁₋₄ alkylamino, CF₃, and halogen;
Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;
Y⁷ is hydrogen, amino, C₁₋₄ alkylamino, C₃₋₆ cycloalkylamino, or di(C₁₋₄ alkyl)amino;
Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;
Y⁹ is O—Y¹⁰, hydroxy, or O—P(═W)O₂H₂, or a linkage to a further of said nucleoside units of said compound;
Y¹⁰ is a conjugate molecule or a reporter molecule;
each Y¹¹ is independently H or C₁₋₆ alkyl;
Y¹² and Y¹³ are each independently hydrogen C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl;
Y¹⁴ is H, CF₃, C₁₋₄ alkyl, amino, C₁₋₄ alkylamino, C₃₋₆ cycloalkylamino, or di(C₁₋₄ alkyl)amino; and
at least one of Y¹, Y², Y³, Y⁴ or Y⁹ is a linkage to a further of said nucleoside units of said compound.

2. A compound of claim 1 wherein said plurality of linked nucleoside units comprises an oligonucleotide, the nucleosides of said oligonucleotide linked together by phosphodiester, phosphorothioate, chiral phosphorothioate, phosphorodithioate, phosphotriester, aminoalkylphosphotriester, methyl or alkyl phosphonate, 3′-alkylene phosphonate, 5′-alkylene phosphonate, chiral phosphonate, phosphonate, 3′-amino phosphoramidate, aminoalkylphosphoramidate, thionophosphoramidate, thionoalkyl-phosphonate, thionoalkylphosphotriester, selenophosphates or boranophosphate linkages.

3. A compound of claim 2 wherein one of said linkages comprise an inverted internucleotide linkages that is a 3′ to 3′ or 5′ to 5′ linkage.

4. A compound of claim 3 wherein said inverted polarity linkage comprises a single 3′ to 3′ linkage at the 3′-most internucleotide linkage of said compound.

5. A compound of claim 1 wherein said plurality of linked nucleoside units comprises an oligonucleoside, the nucleosides of said oligonucleoside linked together by morpholino, siloxane, sulfide, sulfoxide, sulfone; formacetal, thioformacetal, methylene formacetal, methylene thioformacetal, riboacetal, alkene, sulfamate, methyleneimino, methylenehydrazino, sulfonate, sulfonamide or amide linkages.

6. A compound of claim 1 wherein said plurality of linked nucleoside units comprise a chimeric oligonucleotide having a first region capable of serving as a substrate for an RNA cleaving enzyme and a second region containing said nucleoside of structural formula I.

7. A compound of claim 6 wherein said RNA cleaving enzyme is an RNase H enzyme.

8. A compound of claim 6 wherein said RNA cleaving enzyme is a dsRNase.

9. A compound of claim 1 wherein a further of said linked nucleoside units comprises a 2′-deoxy nucleoside.

10. A compound of claim 1 wherein a further of said linked nucleoside units comprises a 2′-ribonucleoside.

11. A compound of claim 1 wherein a further of said linked nucleoside units comprise a nucleoside having a 2′ substituent group and wherein said substituent group is C₁-C₂₀ alkyl, C₂-C₂₀ alkenyl, C₂-C₂₀ alkynyl, C₅-C₂₀ aryl, —O-alkyl, —O—alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—R), carboxyl (—C(═O)OH), nitro (—NO₂), nitroso (—N═O), cyano (—CN), trifluoromethyl (—-CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—R), sulfone (—S(═O)₂—R), disulfide (—S—S—R), silyl, heterocycle, carbocycle, intercalator, reporter group, conjugate, polyamine, polyamide, polyalkylene glycol, and polyethers of the formula (—O—alkyl)ₘ, where m is 1 to about 10; wherein each R is, independently, hydrogen, a protecting group or substituted or unsubstituted alkyl, alkenyl, or alkynyl wherein said substituted alkyl, alkenyl, or alkynyl are substituted with haloalkyl, alkenyl, alkoxy, thioalkoxy, haloalkoxy, aryl groups as well as halogen, hydroxyl, amino, azido, carboxy, cyano, nitro, mercapto, sulfides, sulfones, or sulfoxides.

12. A compound of claim 11 wherein said 2′ substituent group O—CH₂—CH₂—O—CH₃.

13. A compound of claim 1 wherein Y¹ is alkyl unsubstituted or substituted with hydroxy, amino, C₁₋₄ alkoxy, C₁₋₄ alkylthio, or one to three fluorine atoms.

14. A compound of claim 13 wherein Y¹ is methyl or trifluoromethyl.

15. A compound of claim 1 wherein Y¹ is alkyl unsubstituted or substituted with hydroxy, amino, C₁₋₄ alkoxy, C₁₋₄ alkylthio, or one to three fluorine atoms; and
Y² is hydrogen, fluorine, methoxy or hydroxyl.

16. A compound of claim 15 wherein Y² is hydrogen or hydroxyl.

17. An antisense oligonucleotide comprising a compound of claim 1.

18. A ribozyme comprising a compound of claim 1.

19. An aptamers comprising a compound of claim 1.

20. A substrate strand for a RNase H or a RNA dsRNase cleaving enzyme comprising a compound of claim 1.

21. A siRNA molecule having first and second strands, at least one of said strands comprising compound of claim 1.

22. A nucleic acid probe comprising a compound of claim 1.

23. A PCR primer comprising a compound of claim 1.

24. A diagnostic oligonucleotide comprising a compound of claim 1.

25. A compound of claim 1 wherein Y⁹ is a linkage to a further of said nucleoside units of said compound, and
at least one of Y¹, Y², Y³ or Y⁴ is a linkage to a further of said nucleoside units of said compound.

26. A compound comprising a plurality of linked nucleoside units, at least one of said nucleoside units comprising a modified nucleoside of structural formula I of the indicated stereochemical configuration: 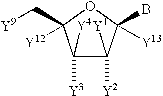
wherein B is selected from the group consisting of 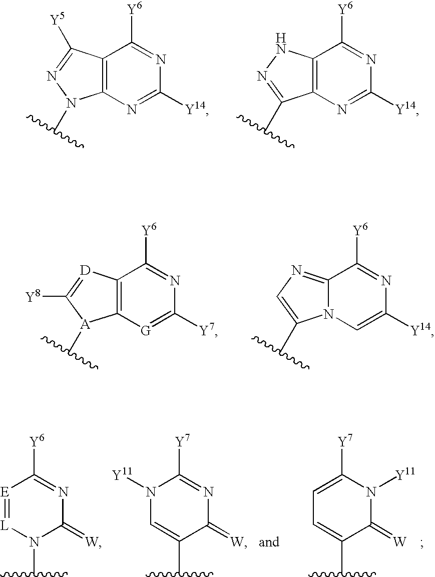
A is N or CH;
G is N or CH;
D is N;
E is N or CY⁵;
L is N or CY⁵;
W is O or S;
Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ to alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;
one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y⁵ is H, C₁₋₆ alkyl, C₂₋₆ alkenyl, C₂₋₆ alkynyl, C₁₋₄ alkylamino, CF₃, and halogen;
Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;
Y⁷ is hydrogen, amino, C₁₋₄ alkylamino, C₃₋₆ cycloalkylamino, or di(C₁₋₄ alkyl)amino;
Y⁸ is H, halogen, CN, carboxy, C₁₋₄ alkyloxycarbonyl, N₃, amino, C₁₋₄ alkylamino, di(C₁₋₄ alkyl)amino, hydroxy, C₁₋₆ alkoxy, C₁₋₆ alkylthio, C₁₋₆ alkylsulfonyl, or (C₁₋₄ alkyl)₀₋₂ aminomethyl;
Y⁹ is O—Y¹⁰, hydroxy, or O—P(═W)O₂H₂, or a linkage to a further of said nucleoside units of said compound;
Y¹⁰ is a conjugate molecule or a reporter molecule;
each Y¹¹ is independently H or C₁₋₆ alkyl;
Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl; and
Y¹⁴ is H, CF₃, C₁₋₄ alkyl, amino, C₁₋₄ alkylamino, C₃₋₆ cycloalkylamino, or di(C₁₋₄ alkyl)amino.

27. A compound of the structures: 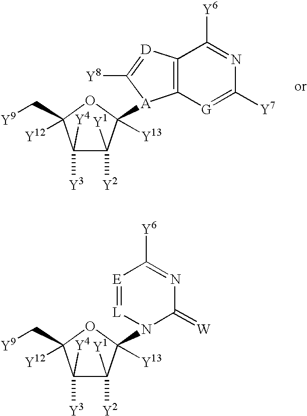
wherein
A is N or CH;
G is N or CH;
D is N;
E is N or CY⁵;
L is N or CY⁵;
W is O or S;
Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;
one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;
Y⁷ is hydrogen, amino, C₁₋₄ alkylamino, C₃₋₆ cycloalkylamino, or di(C₁₋₄ alkyl)amino;
Y⁹ is O—Y¹⁰, hydroxy, or O—P(═W)O₂H₂, or a linkage to a further of said nucleoside units of said compound;
Y¹⁰ is a conjugate molecule or a reporter molecule;
each Y¹¹ is independently H or C₁₋₆ alkyl; and
Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl.

28. A compound of claim 27 where one of Y¹ and Y² is methyl and the other of Y¹ and Y² is hydroxyl or halogen.

29. A compound of claim 27 of the structure: 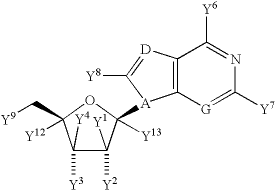
wherein A is N or CH;
G is N or CH;
D is N;
W is O or S;
Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;
one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;
Y⁷ is hydrogen, amino, C₁₋₄ alkylamino, C₃₋₆ cycloalkylamino, or di(C₁₋₄ alkyl)amino;
Y⁹ is O—Y¹⁰, hydroxy, or O—P(═W)O₂H₂, or a linkage to a further of said nucleoside units of said compound;
Y¹⁰ is a conjugate molecule or a reporter molecule;
each Y¹¹ is independently H or C₁₋₆ alkyl; and
Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl.

30. A compound of claim 29 where one of Y¹ and Y² is methyl and the other of Y¹ and Y² is hydroxyl or halogen.

31. A compound of claim 27 of the structure: 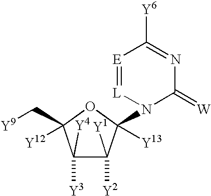
wherein E is N or CY⁵;
L is N or CY⁵;
W is O or S;
Y¹ is hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y² is hydrogen, hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰; provide that Y2 is not hydrogen when Y1 is fluoro or hydroxyl;
one of Y3 or Y4 is a linkage to a further of said nucleoside units of said compound and the other of Y3 or Y4 is hydrogen; hydroxyl; halogen; C₂₋₄ alkenyl, C₂₋₄ alkynyl, or C₁₋₄ alkyl optionally substituted with amino, hydroxy, or 1 to 3 fluorine atoms; C₁₋₁₀ alkoxy, optionally substituted with C₁₋₃ alkoxy, C₁₋₃ thioalkoxy or 1 to 3 fluorine atoms; C₂₋₆ alkenyloxy; C₁₋₄ alkylthio; C₁₋₈ alkylcarbonyloxy; aryloxycarbonyl; azido; amino; C₁₋₄ alkylamino; di(C₁₋₄ alkyl)amino; or Y¹⁰;
Y⁶ is H, OH, SH, NH₂, C₁₋₄ alkylamino, di(C₁₋₄ alkyl)amino, C₃₋₆ cycloalkylamino, halogen, C₁₋₄ alkyl, C₁₋₄ alkoxy, or CF₃;
Y⁹ is O—Y¹⁰, hydroxy, or O—P(═W)O₂H₂, or a linkage to a further of said nucleoside units of said compound;
Y₁₀ is a conjugate molecule or a reporter molecule;
each Y¹¹ is independently H or C₁₋₆ alkyl; and
Y¹² and Y¹³ are each independently hydrogen; C₂₋₄ alkenyl, C₂₋₄ alkynyl, or C₁₋₄ alkyl optionally substituted with amino, hydroxy, or 1 to 3 fluorine atoms; C¹⁻¹⁰ alkoxy, optionally substituted with C₁₋₃ alkoxy, C₁₋₃ thioalkoxy or 1 to 3 fluorine atoms; C₂₋₆ alkenyloxy; C₁₋₄ alkylthio; or Y¹² and Y² together with the carbon atom to which they are attached form a 3- to 6-membered saturated monocyclic ring system optionally containing a heteroatom selected from O, S, and NC₀₋₄ alkyl.

32. A compound of claim 31 where one of Y¹ and Y² is methyl and the other of Y¹ and Y² is hydroxyl or halogen.
